(19) United States
(12) Patent Application Publication
(10) Pub. No.: US 2023/0312458 A1
(43) Pub. Date: Oct. 5, 2023

(54) COMPOUND, LIGHT-EMITTING DEVICE, LIGHT-EMITTING APPARATUS, ELECTRONIC DEVICE, AND ALIGHTING DEVICE

(71) Applicant: SEMICONDUCTOR ENERGY LABORATORY CO., LTD., Atsugi-shi, Kanagawa-ken (JP)

(72) Inventors: Takuya HARUYAMA, Atsugi, Kanagawa (JP); Nobuharu OHSAWA, Zama, Kanagawa (JP); Satoshi SEO, Sagamihara, Kanagawa (JP)

(21) Appl. No.: 18/003,702

(22) PCT Filed: Jun. 24, 2021
(86) PCT No.: PCT/IB2021/055583

§ 371 Date: Dec. 29, 2022

(30) Foreign Application Priority Data
Jul. 3, 2020 (JP) 2020-115546

(51) Int. Cl. C07C 211/61; H10K 85/60; C09K 11/06; C09K 11/02

(52) U.S. Cl. CPC C07C 211/61; H10K 85/615; H10K 85/633; C09K 11/06; C09K 11/02; H10K 2101/20

(57) ABSTRACT

A novel compound is provided. The compound is represented by General Formula (G1).

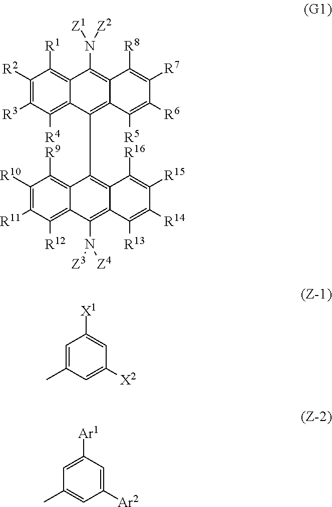

In General formula (G1), each of Z¹ to Z⁴ independently has a structure represented by General formula (Z-1) or General formula (Z-2). In General Formula (Z-1), each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of Ar¹ and Ar² independently represents an aromatic hydrocarbon group having 6 to 13 carbon atoms, and at least one of Ar¹ and Ar² includes a substituent that is the same as X¹.

### TECHNICAL FIELD

One embodiment of the present invention relates to a compound, a light-emitting device, a light-emitting apparatus, an electronic device, and a lighting device. However, one embodiment of the present invention is not limited thereto. That is, one embodiment of the present invention relates to an object, a method, a manufacturing method, or a driving method. Alternatively, one embodiment of the present invention relates to a process, a machine, manufacture, or a composition of matter.

### BACKGROUND ART

In recent years, research has been extensively conducted on light-emitting devices utilizing electroluminescence (EL). These light-emitting devices have a structure in which an EL layer (containing a light-emitting substance) is interposed between a pair of electrodes. In a light-emitting device, voltage application between a pair of electrodes causes, in an EL layer, recombination of electrons and holes injected from the electrodes, which brings a light-emitting substance (an organic compound) contained in the EL layer into an excited state; and light is emitted when the light-emitting substance returns to the ground state from the excited state. The excited state can be a singlet excited state (S*) and a triplet excited state (T*); and light emission from a singlet excited state is referred to as fluorescence, and light emission from a triplet excited state is referred to as phosphorescence. The statistical generation ratio thereof in the light-emitting device is considered to be S*:T*=1:3. Therefore, light-emitting devices using phosphorescent substances capable of converting the energy of the triplet excited state into light emission have been actively developed recently to obtain high efficiency.

As a material capable of converting part or all of the energy of the triplet excited state into light emission, a thermally activated delayed fluorescent (TADF) material is known in addition to a phosphorescent substance. In the TADF material, a singlet excited state can be generated from a triplet excited state by reverse intersystem crossing.

A method in which in a light-emitting device containing a TADF material and a fluorescent substance in combination, the singlet excitation energy of the TADF material is transferred to the fluorescent substance and light emission is efficiently obtained from the fluorescent substance has been proposed (see Patent Document 1).

### REFERENCE

### Patent Document

As for energy transfer from a host material to a guest material in a light-emitting layer of a light-emitting device, in general it is preferable that the concentration ratio of the guest material (fluorescent substance) to the host material be increased in order to increase the efficiency of energy transfer due to the Förster mechanism; however, it is known that there is a trade-off relationship: an increase in the concentration ratio of the guest material increases the rate of energy transfer due to the Dexter mechanism, which results in a decrease in the emission efficiency. Therefore, increasing the concentration ratio of the guest material has not been an effective means for improving the emission efficiency.

### REFERENCE

### Patent Document

[Patent Document 1] Japanese Published Patent Application No. 2014-45179

### SUMMARY OF THE INVENTION

### Problems to be Solved by the Invention

Thus, in one embodiment of the present invention, a novel compound is provided. Another embodiment provides a novel compound that efficiently receives energy from a singlet excited state (S*) (hereinafter the energy is referred to as singlet excitation energy) of a host material even when the concentration ratio of the guest material in an EL layer of a light-emitting device is increased, whereby the transfer of energy from a triplet excited state (T*) (the energy is hereinafter referred to as triplet excitation energy) of the host material is unlikely to occur (energy transfer due to the Dexter mechanism can be prevented).

In one embodiment of the present invention, a novel compound that can be used in a light-emitting device is provided. In one embodiment of the present invention, a novel compound that can be used in an EL layer of a light-emitting device is provided. In addition, a novel light-emitting device with high emission efficiency using a novel compound of one embodiment of the present invention is provided. In addition, a novel light-emitting apparatus, a novel electronic device, or a novel lighting device is provided.

Note that the description of these objects does not preclude the existence of other objects. In one embodiment of the present invention, there is no need to achieve all of these objects.

Objects other than these are apparent from the description of the specification, the drawings, the claims, and the like, and objects other than these can be derived from the description of the specification, the drawings, the claims, and the like.

### Means for Solving the Problems

One embodiment of the present invention is a fluorescent substance and a compound represented by General Formula (G1) below.

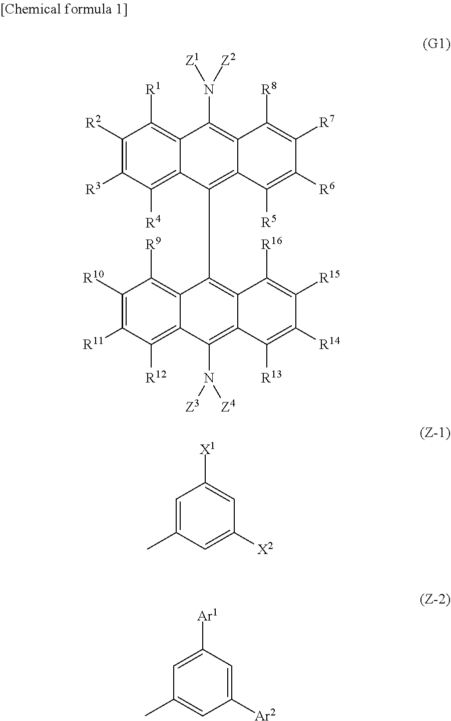

In General formula (G1) above, each of Z¹ to Z⁴ independently has a structure represented by General formula (Z-1) or General formula (Z-2). In General Formula (Z-1), each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of Ar¹ and Ar² independently represents an aromatic hydrocarbon group having 6 to 13 carbon atoms, and at least one of Ar¹ and Ar² includes a substituent that is the same as X¹. Moreover, each of R¹ to R¹⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

Another embodiment of the present invention is a compound represented by General Formula (G2) below.

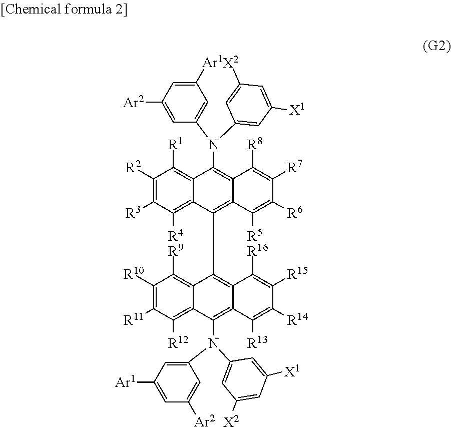

In General Formula (G2) above, each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of Ar¹ and Ar² independently represents an aromatic hydrocarbon group having 6 to 13 carbon atoms, and at least one of Ar¹ and Ar² includes a substituent that is the same as X¹. Moreover, each of R¹ to R¹⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

Another embodiment of the present invention is a compound represented by General Formula (G3) below.

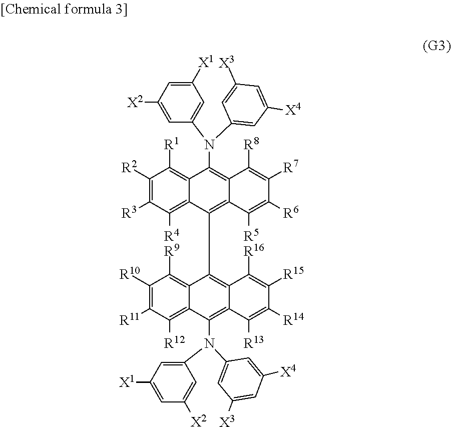

In General formula (G3) above, each of X¹ to X⁴ independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of R¹ to R¹⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

Another embodiment of the present invention is a compound represented by General Formula (G4) below.

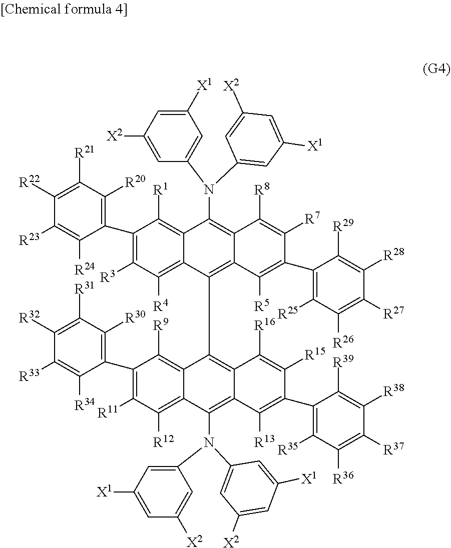

In General formula (G4) above, each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of R¹, R³ to R⁵, R⁷ to R⁹, RH to R¹³, R¹⁵ to R¹⁶, and R²⁰ to R³⁹ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

Another embodiment of the present invention is a compound represented by Structural Formula (100) or Structural Formula (101).

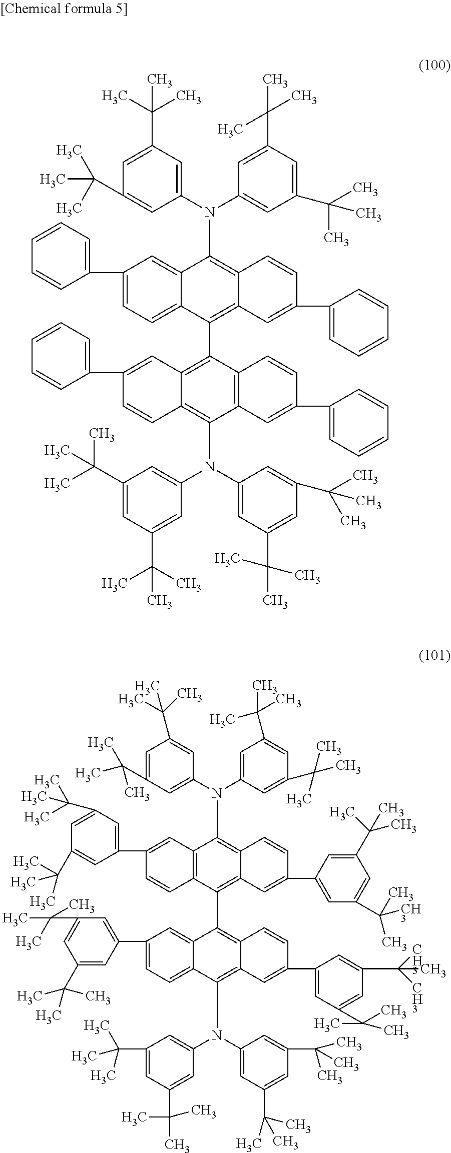 [SMILES: CC(C)(C)c1cc(N(c2cc(C(C)(C)C)cc(C(C)(C)C)c2)c2c3ccc(-c4ccccc4)cc3c(-c3c4cc(-c5ccccc5)ccc4c(N(c4cc(C(C)(C)C)cc(C(C)(C)C)c4)c4cc(C(C)(C)C)cc(C(C)(C)C)c4)c4cc(-c5ccccc5)ccc34)c3ccc(-c4ccccc4)cc23)cc(C(C)(C)C)c1 ; CC(C)(C)c1cc(-c2ccc3c(N(c4cc(C(C)(C)C)cc(C(C)(C)C)c4)c4cc(C(C)(C)C)cc(C(C)(C)C)c4)c4cc(-c5cc(C(C)(C)C)cc(C(C)(C)C)c5)ccc4c(-c4c5cc(-c6cc(C(C)(C)C)cc(C(C)(C)C)c6)ccc5c(N(c5cc(C(C)(C)C)cc(C(C)(C)C)c5)c5cc(C(C)(C)C)cc(C(C)(C)C)c5)c5cc(-c6cc(C(C)(C)C)cc(C(C)(C)C)c6)ccc45)c3c2)cc(C(C)(C)C)c1]

Another embodiment of the present invention is a light-emitting device using the above-described compound of one embodiment of the present invention. Note that the present invention also includes a light-emitting device that is formed using the compound of one embodiment of the present invention for an EL layer between a pair of electrodes or a light-emitting layer included in the EL layer. In addition to the above-described light-emitting devices, the present invention includes a light-emitting device including a layer (e.g., a cap layer) that is in contact with an electrode and contains an organic compound. In addition to the light-emitting devices, a light-emitting apparatus including a transistor, a substrate, and the like is also included in the scope of the invention. Furthermore, in addition to the light-emitting apparatus, an electronic device and a lighting device that include a microphone, a camera, an operation button, an external connection portion, a housing, a cover, a support, a speaker, or the like are also included in the scope of the invention.

In addition, the scope of one embodiment of the present invention includes a light-emitting apparatus including a light-emitting device, and a lighting device including the light-emitting apparatus. Accordingly, a light-emitting apparatus in this specification refers to an image display device or a light source (including a lighting device). In addition, a light-emitting apparatus includes a module in which a light-emitting apparatus is connected to a connector such as an FPC (Flexible Printed Circuit) or a TCP (Tape Carrier Package), a module in which a printed wiring board is provided on the tip of a TCP, or a module in which an IC (integrated circuit) is directly mounted on a light-emitting device by a COG (Chip On Glass) method.

### Effect of the Invention

One embodiment of the present invention can provide a novel compound. In one embodiment of the present invention, a novel compound that can be used in a light-emitting device can be provided. In one embodiment of the present invention, a novel compound that can be used in an EL layer of a light-emitting device can be provided. A light-emitting device with high emission efficiency can be provided. A light-emitting device with high reliability can be provided.

A novel light-emitting device can be provided. In one embodiment of the present invention, a novel light-emitting apparatus, a novel electronic device, or a novel lighting device can be provided.

Note that the description of these effects does not preclude the existence of other effects. In one embodiment of the present invention, there is no need to achieve all of these effects. Effects other than these are apparent from the description of the specification, drawings, claims, and the like and effects other than these can be derived from the description of the specification, drawings, claims, and the like.

### BRIEF DESCRIPTION OF THE DRAWINGS

FIG. 1A is a diagram showing a structure of a light-emitting device. FIG. 1B is a diagram illustrating a light-emitting layer.

FIG. 2A is a conceptual diagram of energy transfer between a typical guest material and a host material. FIG. 2B is a conceptual diagram of energy transfer between a compound (guest material) of one embodiment of the present invention and a host material.

FIG. 3A is a conceptual diagram of energy transfer between compounds in a light-emitting layer.

FIG. 3B is a conceptual diagram of energy transfer between compounds in a light-emitting layer.

FIG. 3C is a conceptual diagram of energy transfer between compounds in a light-emitting layer.

FIG. 4A is a conceptual diagram of energy transfer between compounds in a light-emitting layer.

FIG. 4B is a conceptual diagram of energy transfer between compounds in a light-emitting layer.

FIG. 4C is a conceptual diagram of energy transfer between compounds in a light-emitting layer.

FIG. 5A is a conceptual diagram of energy transfer between compounds in a light-emitting layer.

FIG. 5B is a conceptual diagram of energy transfer between compounds in a light-emitting layer.

FIG. 6A and FIG. 6B are diagrams illustrating structures of light-emitting devices.

FIG. 7A, FIG. 7B, and FIG. 7C are diagrams illustrating light-emitting apparatuses.

FIG. 8A is a top view illustrating a light-emitting apparatus. FIG. 8B is a cross-sectional view illustrating the light-emitting apparatus.

FIG. 9A is a diagram illustrating a mobile computer. FIG. 9B is a diagram illustrating a portable image reproducing device. FIG. 9C is a diagram illustrating a digital camera. FIG. 9D is a diagram illustrating a portable information terminal. FIG. 9E is a diagram illustrating a portable information terminal. FIG. 9F is a diagram illustrating a television device. FIG. 9G is a diagram illustrating a portable information terminal.

FIG. 10A, FIG. 10B, and FIG. 10C are diagrams illustrating a foldable portable information terminal.

FIG. 11A and FIG. 11B are diagrams illustrating an automobile.

FIG. 12 is a diagram illustrating a light-emitting device.

FIG. 13 is a diagram illustrating a light-emitting device.

FIG. 14 is a ¹H-NMR chart of an organic compound represented by Structural Formula (100).

FIG. 15 shows an ultraviolet-visible absorption spectrum and an emission spectrum of the organic compound represented by Structural Formula (100).

FIG. 16 is a ¹H-NMR chart of an organic compound represented by Structural Formula (101).

FIG. 17 shows an ultraviolet-visible absorption spectrum and an emission spectrum of the organic compound represented by Structural Formula (101).

FIG. 18 is a diagram illustrating a light-emitting device.

FIG. 19 is a graph showing the current density-luminance characteristics of Light-emitting device 1-1, Light-emitting device 1-2, Light-emitting device 1-3, Comparative light-emitting device 1-a, and Comparative light-emitting device 1-b.

FIG. 20 is a graph showing the voltage-luminance characteristics of Light-emitting device 1-1, Light-emitting device 1-2, Light-emitting device 1-3, Comparative light-emitting device 1-a, and Comparative light-emitting device 1-b.

FIG. 21 is a graph showing the luminance-current efficiency characteristics of Light-emitting device 1-1, Light-emitting device 1-2, Light-emitting device 1-3, Comparative light-emitting device 1-a, and Comparative light-emitting device 1-b.

FIG. 22 is a graph showing the voltage-current density characteristics of Light-emitting device 1-1, Light-emitting device 1-2, Light-emitting device 1-3, Comparative light-emitting device 1-a, and Comparative light-emitting device 1-b.

FIG. 23 is a graph showing the luminance-power efficiency characteristics of Light-emitting device 1-1, Light-emitting device 1-2, Light-emitting device 1-3, Comparative light-emitting device 1-a, and Comparative light-emitting device 1-b.

FIG. 24 is a graph showing the luminance-external quantum efficiency characteristics of Light-emitting device 1-1, Light-emitting device 1-2, Light-emitting device 1-3, Comparative light-emitting device 1-a, and Comparative light-emitting device 1-b.

FIG. 25 is a graph showing the electroluminescence spectra of Light-emitting device 1-1, Light-emitting device 1-2, Light-emitting device 1-3, Comparative light-emitting device 1-a, and Comparative light-emitting device 1-b.

FIG. 26 shows reliability measurement results of Light-emitting device 1-1, Light-emitting device 1-2, Light-emitting device 1-3, and Comparative light-emitting device 1-b.

FIG. 27 is a graph showing the current density-luminance characteristics of Light-emitting device 2-1, Light-emitting device 2-2, Light-emitting device 2-3, Light-emitting device 2-4, Comparative light-emitting device 2-a, and Comparative light-emitting device 2-b.

FIG. 28 is a graph showing the voltage-luminance characteristics of Light-emitting device 2-1, Light-emitting device 2-2, Light-emitting device 2-3, Light-emitting device 2-4, Comparative light-emitting device 2-a, and Comparative light-emitting device 2-b.

FIG. 29 is a graph showing the luminance-current efficiency characteristics of Light-emitting device 2-1, Light-emitting device 2-2, Light-emitting device 2-3, Light-emitting device 2-4, Comparative light-emitting device 2-a, and Comparative light-emitting device 2-b.

FIG. 30 is a graph showing the voltage-current density characteristics of Light-emitting device 2-1, Light-emitting device 2-2, Light-emitting device 2-3, Light-emitting device 2-4, Comparative light-emitting device 2-a, and Comparative light-emitting device 2-b.

FIG. 31 is a graph showing the luminance-power efficiency characteristics of Light-emitting device 2-1, Light-emitting device 2-2, Light-emitting device 2-3, Light-emitting device 2-4, Comparative light-emitting device 2-a, and Comparative light-emitting device 2-b.

FIG. 32 is a graph showing the luminance-external quantum efficiency characteristics of Light-emitting device 2-1, Light-emitting device 2-2, Light-emitting device 2-3, Light-emitting device 2-4, Comparative light-emitting device 2-a, and Comparative light-emitting device 2-b.

FIG. 33 is a graph showing the electroluminescence spectra of Light-emitting device 2-1, Light-emitting device 2-2, Light-emitting device 2-3, Light-emitting device 2-4, Comparative light-emitting device 2-a, and Comparative light-emitting device 2-b.

FIG. 34 shows reliability measurement results of Light-emitting device 2-1, Light-emitting device 2-2, Light-emitting device 2-3, Light-emitting device 2-4, and Comparative light-emitting device 2-b.

### MODE FOR CARRYING OUT THE INVENTION

Embodiments of the present invention will be described in detail below with reference to drawings. Note that the present invention is not limited to the following description, and the modes and details of the present invention can be modified in various ways without departing from the spirit and scope of the present invention. Thus, the present invention should not be construed as being limited to the descriptions in the following embodiments.

Note that the position, size, range, or the like of each component shown in drawings and the like is not accurately represented in some cases for easy understanding. Therefore, the disclosed invention is not necessarily limited to the position, size, range, or the like disclosed in drawings and the like.

Furthermore, when describing the structures of the invention with reference to the drawings in this specification and the like, the reference numerals denoting the same components are commonly used in different drawings.

In this specification and the like, a singlet excited state (S*) refers to a singlet state having excitation energy. An S1 level means the lowest level of the singlet excitation energy level, that is, the excitation energy level of the lowest singlet excited state (S1 state). A triplet excited state (T*) refers to a triplet state having excitation energy. A T1 level means the lowest level of the triplet excitation energy level, that is, the excitation energy level of the lowest triplet excited state (T1 state). Note that in this specification and the like, simple expressions singlet excited state and singlet excitation energy level mean the S1 state and the S1 level, respectively, in some cases. In addition, expressions triplet excited state and triplet excitation energy level mean the T1 state and the T1 level, respectively, in some cases.

In this specification and the like, a fluorescent substance refers to a compound that supplies light emission in the visible light region or the near-infrared region when the relaxation from the singlet excited state to the ground state occurs. A phosphorescent substance refers to a compound that supplies light emission in the visible light region or the near-infrared region at room temperature when the relaxation from the triplet excited state to the ground state occurs. In other words, a phosphorescent substance refers to one of compounds that can convert triplet excitation energy into light emission.

### Embodiment 1

In this embodiment, a compound of one embodiment of the present invention is described. The compound of one embodiment of the present invention is represented by General Formula (G1) below.

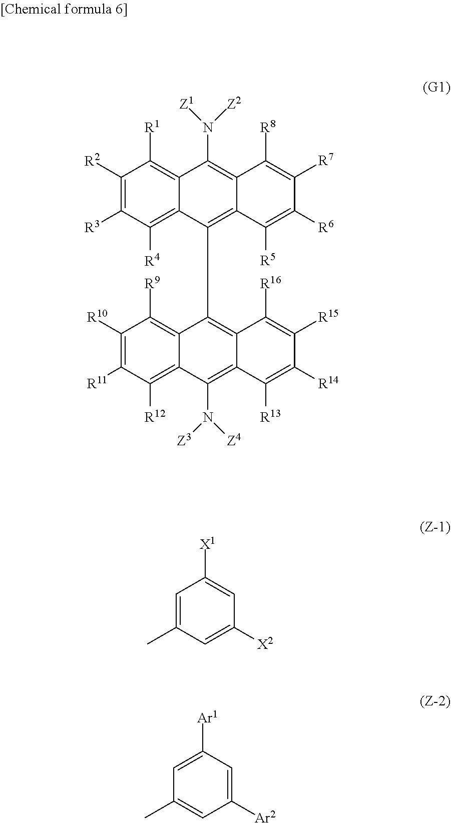

In General Formula (G1), each of Z¹ to Z⁴ independently has a structure represented by General Formula (Z-1) or General Formula (Z-2). In General Formula (Z-1), each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of Ar¹ and Ar² independently represents an aromatic hydrocarbon group having 6 to 13 carbon atoms, and at least one of Art and Ar² includes a substituent that is the same as X¹. Moreover, each of R¹ to R¹⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

Another embodiment of the present invention is a compound represented by General Formula (G2) below.

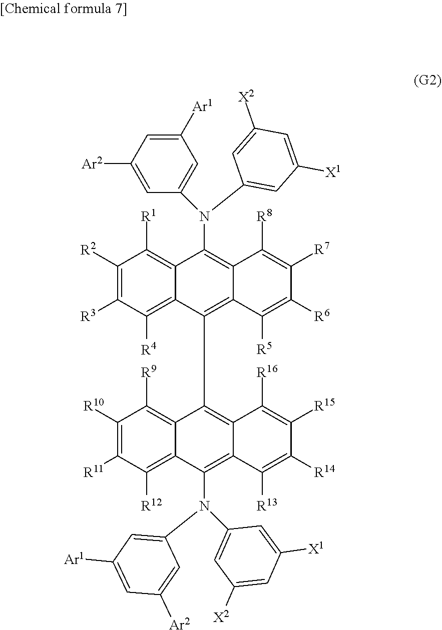

In General Formula (G2), each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of Ar¹ and Ar² independently represents an aromatic hydrocarbon group having 6 to 13 carbon atoms, and at least one of Art and Ar² includes a substituent that is the same as X¹. Moreover, each of R¹ to R¹⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

Another embodiment of the present invention is c compound represented by General Formula (G3) below.

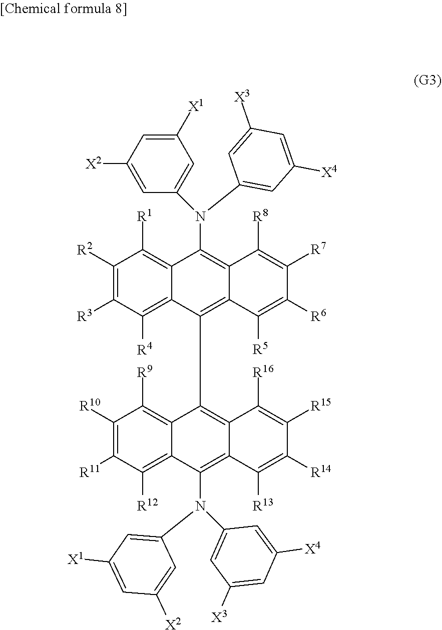

In General formula (G3), each of X¹ to X⁴ independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of R¹ to R¹⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

Another embodiment of the present invention is a compound represented by General Formula (G4) below.

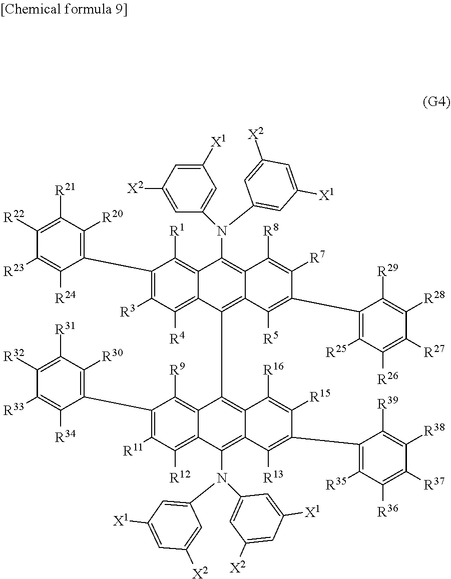

In General Formula (G4), each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of R¹, R³ to R⁵, R⁷ to R⁹, RH to R¹³, R¹⁵ to R¹⁶, and R²⁰ to R³⁹ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

The compound of one embodiment of the present invention is a material having a function of converting singlet excitation energy into light emission (a fluorescent substance), and thus can be used as a guest material in combination with a host material in a light-emitting layer of a light-emitting device. The compound of one embodiment of the present invention has a luminophore that contributes to light emission and a protecting group that prevents the transfer of triplet excitation energy from the host material to the compound due to the Dexter mechanism. The luminophore in the compound of one embodiment of the present invention is a fused aromatic ring or a fused heteroaromatic ring and has a structure in which two or more identical skeletons are bonded. At least two protecting groups are included in each aryl group in two or more diarylamino groups included in the compound of one embodiment of the present invention. Specifically, the protecting group is any of a cycloalkyl group with a bridge structure having 7 to carbon atoms, an alkyl group having 3 to 10 carbon atoms, a substituted or unsubstituted cycloalkyl group having 3 to 10 carbon atoms, and a trialkylsilyl group having 3 to 12 carbon atoms.

In the compound of one embodiment of the present invention, since the luminophore has a structure in which two or more identical skeletons are bonded, the transition dipole moment for light emission increases and hence the molar absorption coefficient increases. This can increase the rate of excitation energy transfer from the host material by the Förster mechanism.

The compound of one embodiment of the present invention has a structure in which the two or more diarylamino groups including the protecting groups are bonded to the luminophore at symmetric positions, whereby the quantum yield can be increased. The use of the diarylamino group in the compound of one embodiment of the present invention inhibits an increase in molecular weight, allowing maintaining the sublimability.

In the compound of one embodiment of the present invention, since the protecting groups are bonded to the aryl groups of the diarylamino groups bonded to the luminophore, the protecting groups can be positioned to cover the luminophore, and the host material and the luminophore can be made away from each other at such a distance that energy transfer from the host material to the luminophore due to the Dexter mechanism is unlikely to occur. Using the aryl groups having the protecting groups improves the effect of covering the luminophore, which makes the above energy transfer due to the Dexter mechanism more unlikely to occur.

In General Formula (G1), General Formula (G2), General Formula (G3), and General Formula (G4) above, as the aromatic hydrocarbon group having 6 to 13 carbon atoms, a phenyl group, a biphenyl group, a naphthyl group, a fluorenyl group, and the like can be given.

In General Formula (G1), General Formula (G2), General Formula (G3), and General Formula (G4) above, specific examples of the alkyl group having 3 to 10 carbon atoms include a propyl group, an isopropyl group, a butyl group, an isobutyl group, a sec-butyl group, a tert-butyl group, a pentyl group, a hexyl group, a heptyl group, and an octyl group.

In General Formula (G1), General Formula (G2), General Formula (G3), and General Formula (G4) above, specific examples of the cycloalkyl group having 3 to 10 carbon atoms include a cyclopropyl group, a cyclobutyl group, and a cyclohexyl group. In the case where the cycloalkyl group has a substituent, specific examples of the substituent include an alkyl group having 1 to 7 carbon atoms, such as a methyl group, an ethyl group, a propyl group, an isopropyl group, a butyl group, an isobutyl group, a sec-butyl group, a tert-butyl group, a pentyl group, or a hexyl group, a cycloalkyl group having 5 to 7 carbon atoms, such as a cyclopentyl group, a cyclohexyl group, a cycloheptyl group, or a 8,9,10-trinorbornanyl group, and an aryl group having 6 to 12 carbon atoms, such as a phenyl group, a naphthyl group, or a biphenyl group.

In General Formula (G1), General Formula (G2), General Formula (G3), and General Formula (G4) above, specific examples of the cycloalkyl group with a bridge structure having 7 to 10 carbon atoms include an adamantyl group, a bicyclo[2.2.1]heptyl group, a tricyclo[5.2.1.02′6]decanyl group, and a noradamantyl group.

In General Formula (G1), General Formula (G2), General Formula (G3), and General Formula (G4) above, specific examples of the trialkylsilyl group having 3 to 12 carbon atoms include a trimethylsilyl group, a triethylsilyl group, and a tert-butyl dimethylsilyl group.

In General Formula (G1), General Formula (G2), General Formula (G3), and General Formula (G4) above, in the case where any of the aromatic hydrocarbon group having 6 to 13 carbon atoms, the cycloalkyl group having 3 to 10 carbon atoms, and the aryl group having 6 to carbon atoms has a substituent, examples of the substituent include an alkyl group having 1 to 7 carbon atoms, such as a methyl group, an ethyl group, a propyl group, an isopropyl group, a butyl group, an isobutyl group, a sec-butyl group, a tert-butyl group, a pentyl group, or a hexyl group, a cycloalkyl group having 5 to 7 carbon atoms, such as a cyclopentyl group, a cyclohexyl group, a cycloheptyl group, or a 8,9,10-trinorbornanyl group, and an aryl group having 6 to 12 carbon atoms, such as a phenyl group, a naphthyl group, or a biphenyl group.

In General Formula (G1), General Formula (G2), General Formula (G3), and General Formula (G4) above, specific examples of the aryl group having 6 to 25 carbon atoms include a phenyl group, a naphthyl group, a biphenyl group, a fluorenyl group, and a spirofluorenyl group. In the case where the aryl group has a substituent, examples of the substituent include the alkyl group having 3 to 10 carbon atoms, the substituted or unsubstituted cycloalkyl group having 3 to carbon atoms, and the trialkylsilyl group having 3 to 12 carbon atoms, which are described above.

Specific examples of the compounds represented by General Formulae (G1) to General Formula (G4) are shown in Structural Formulae (100) to (131) below. Note that specific examples of the compounds shown in General Formula (G1) to General Formula (G4) are not limited to those shown below.

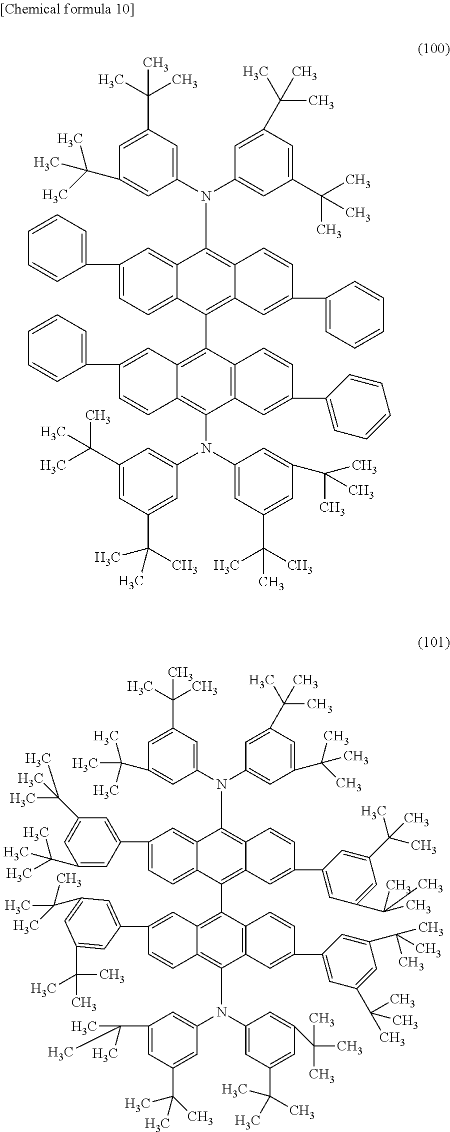

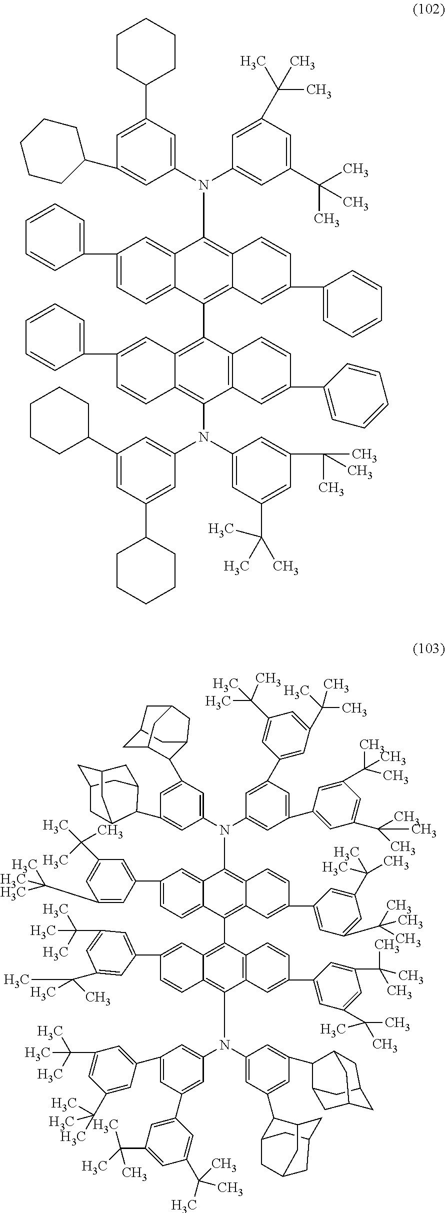

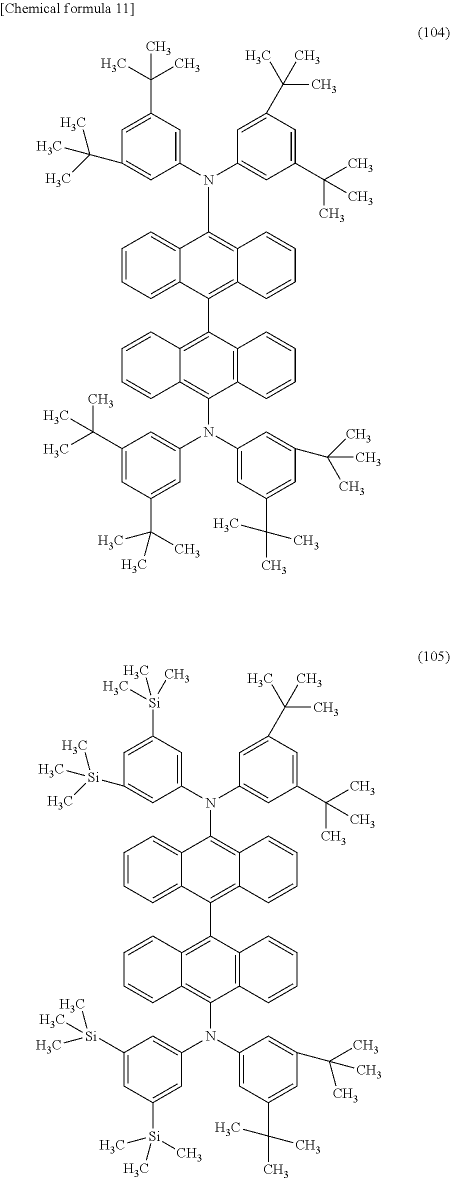

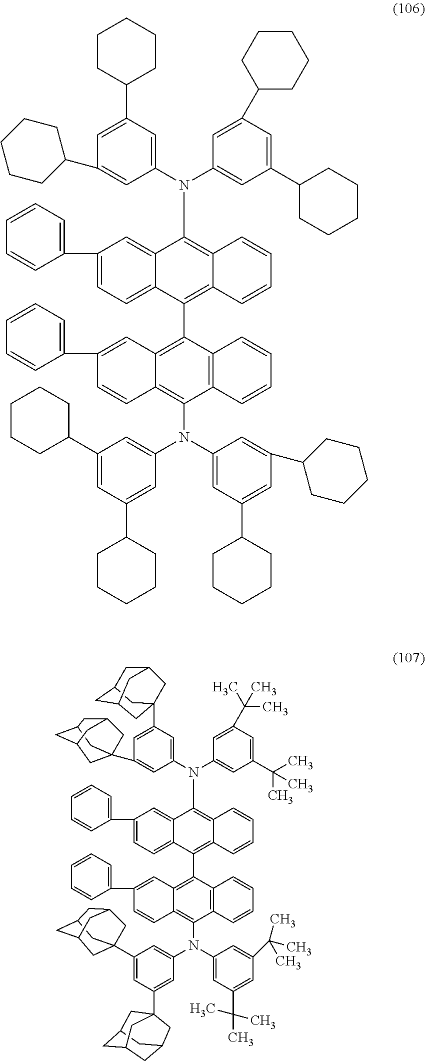

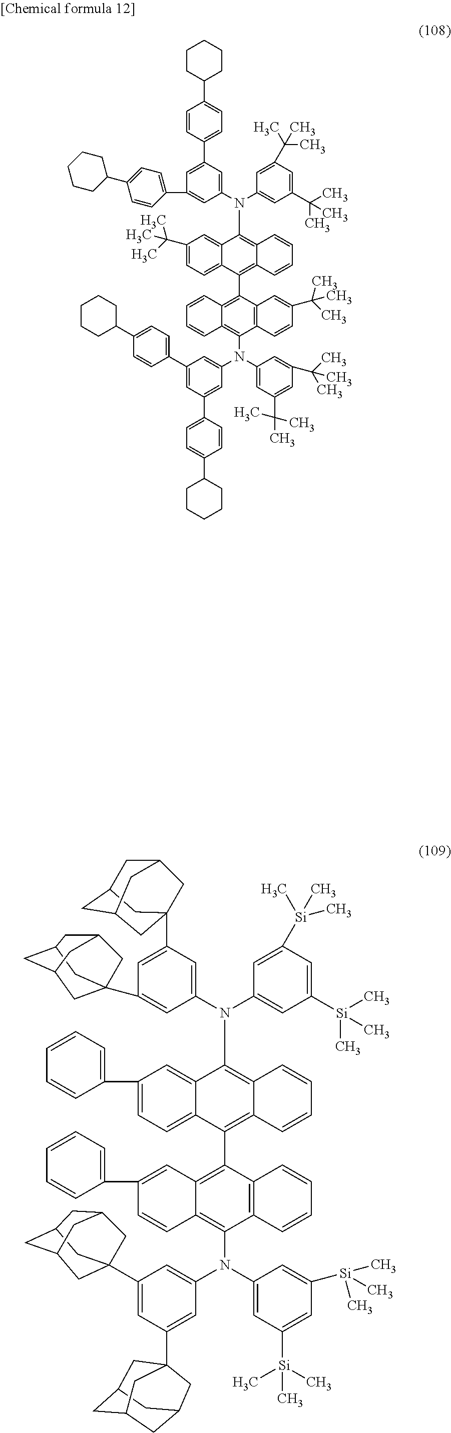

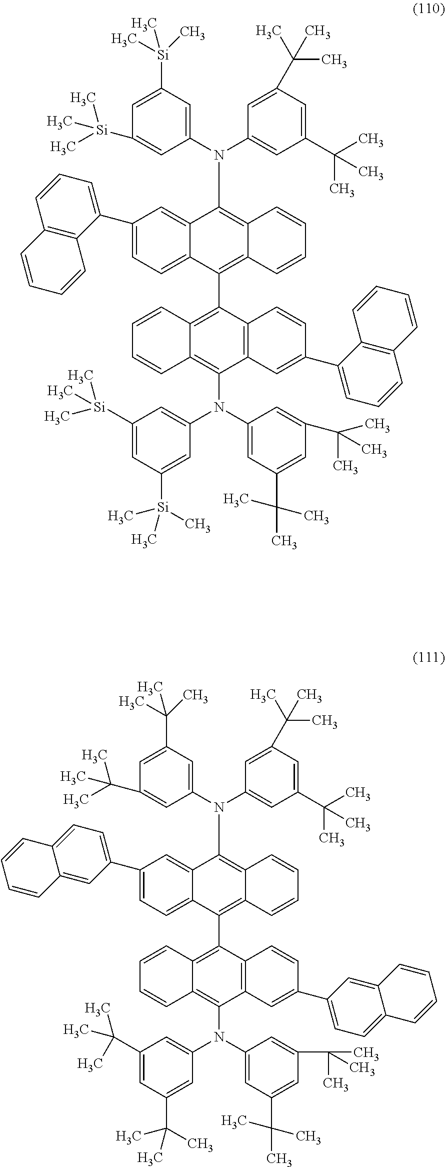

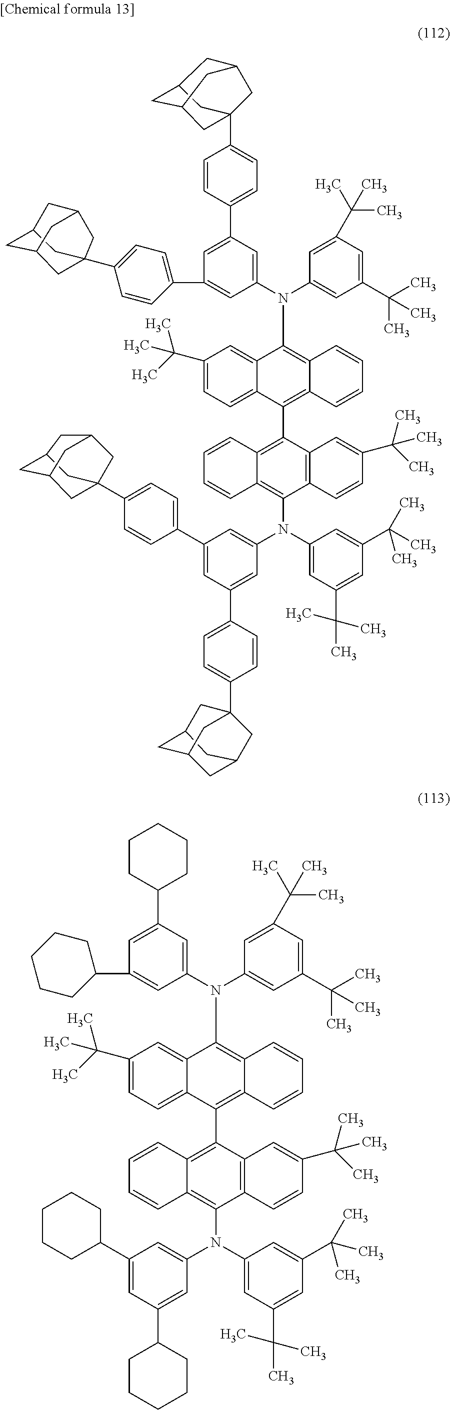

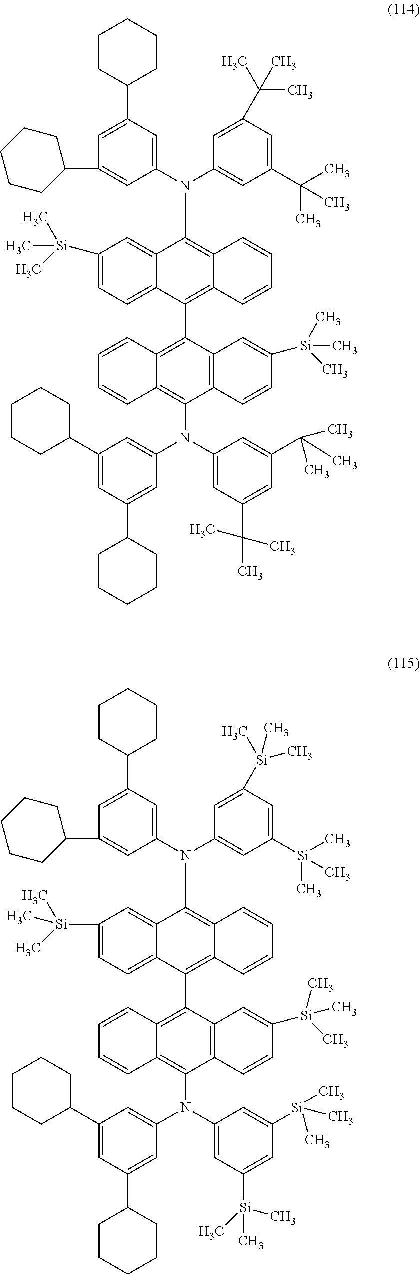

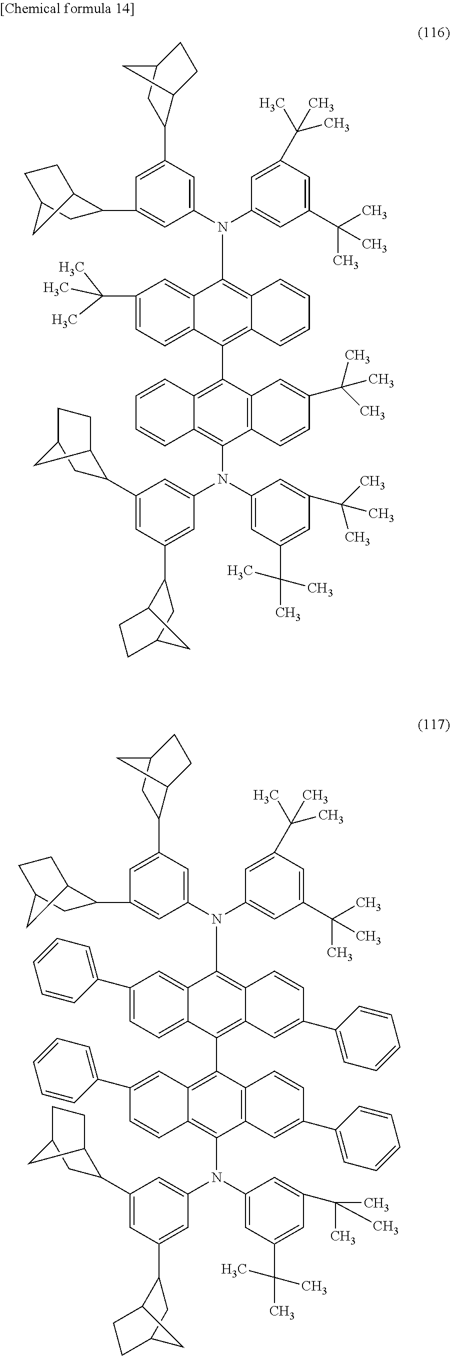

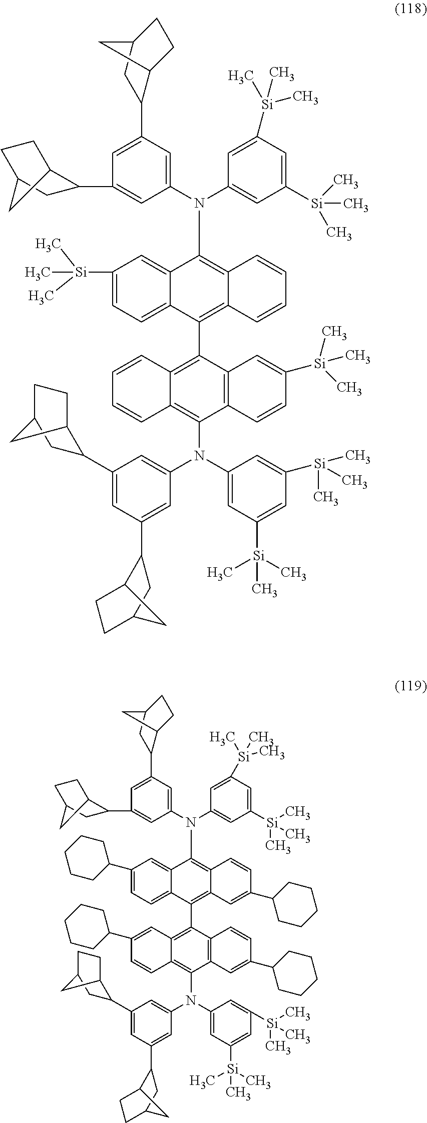

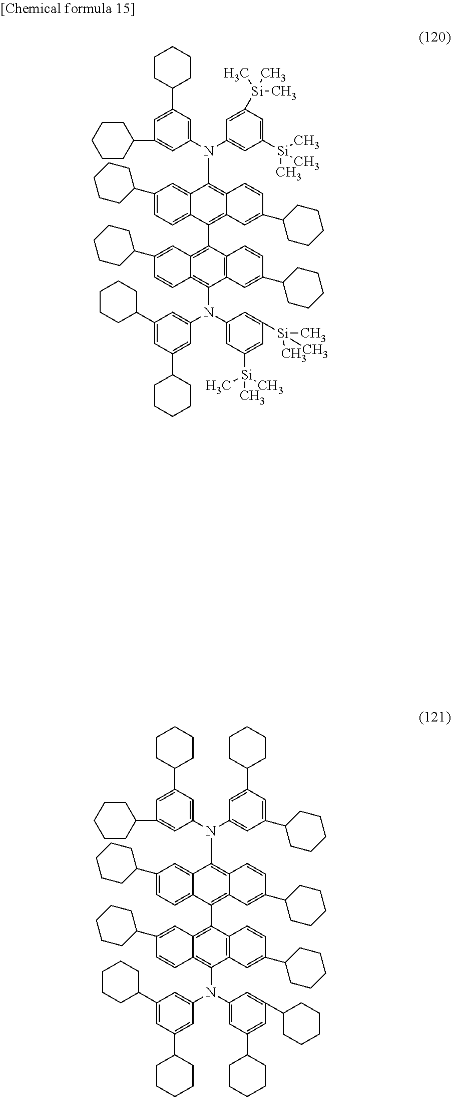

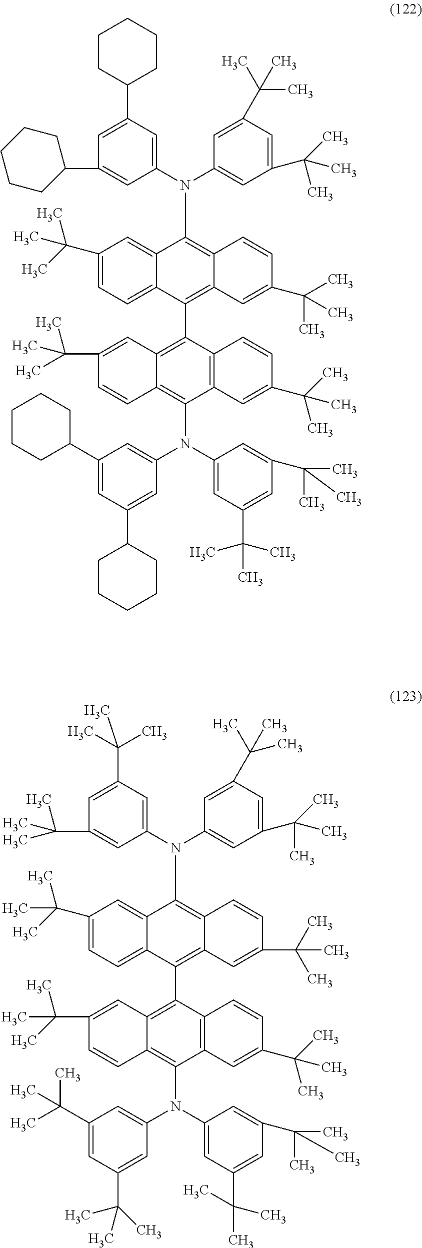

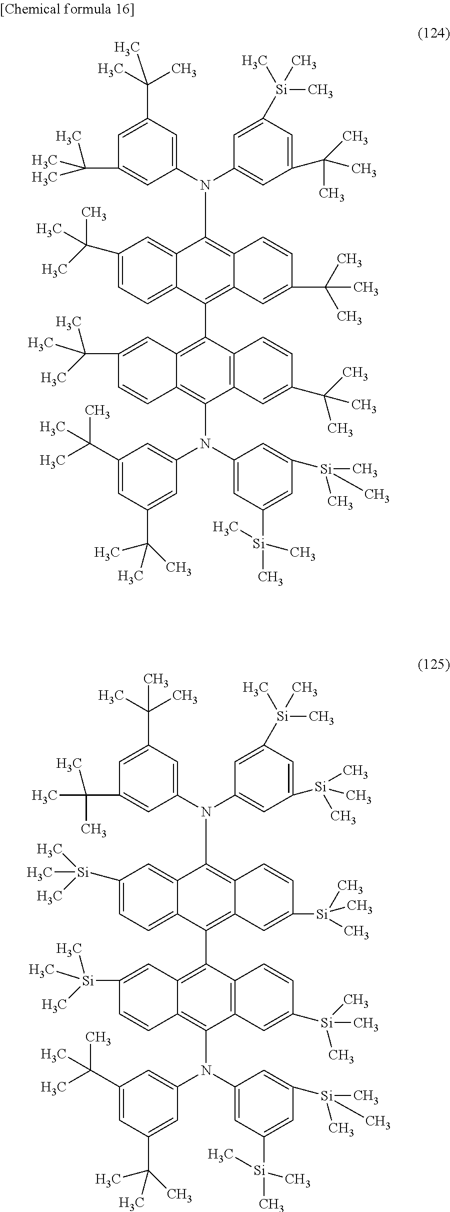

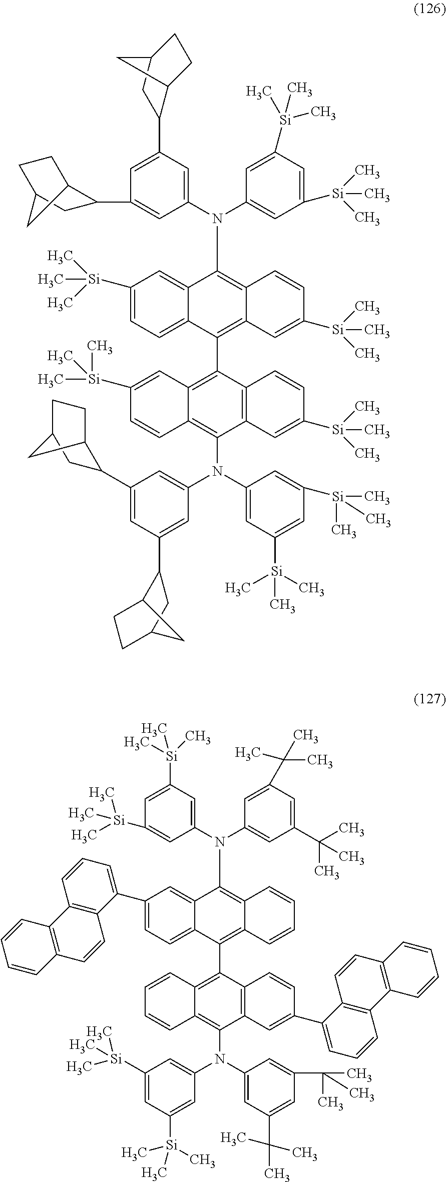

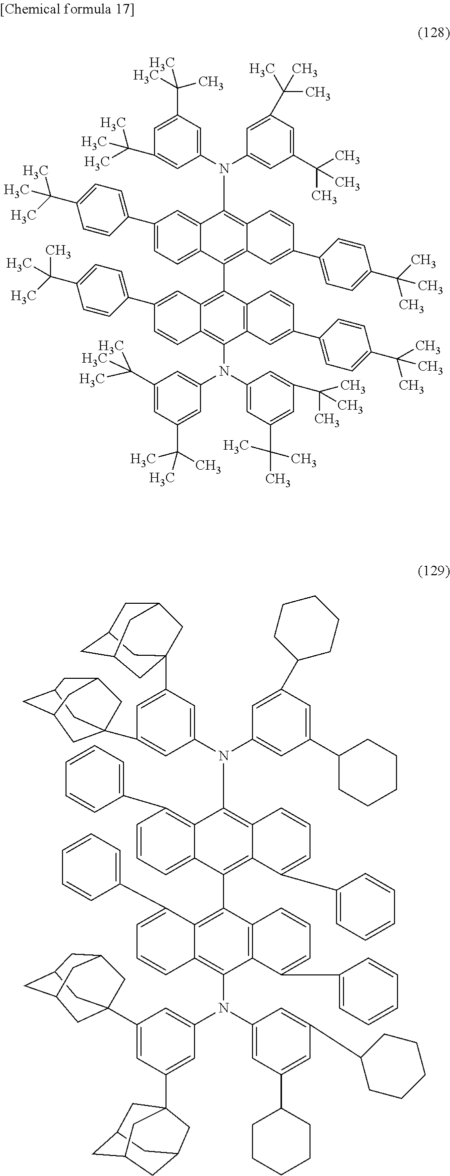

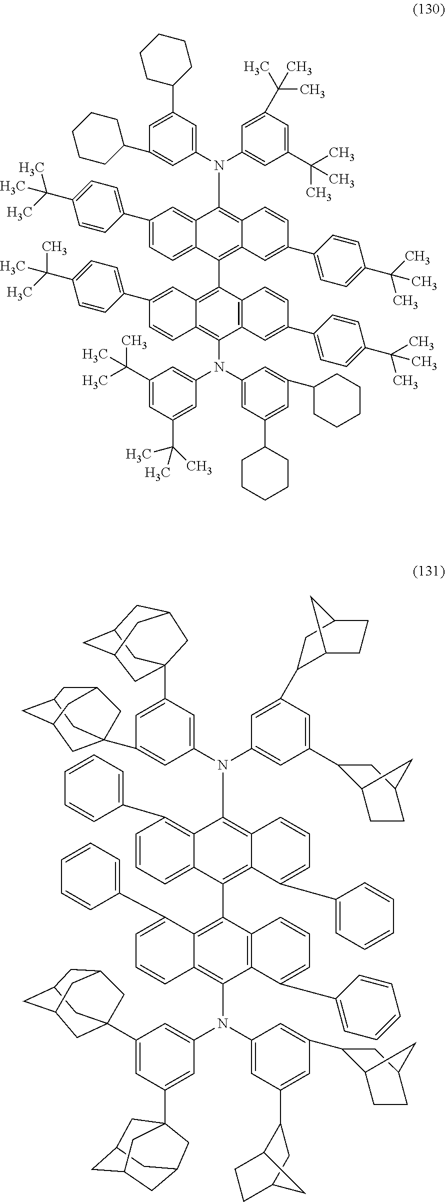

### Synthesis Method of Organic Compound Represented by General Formula (G1)

Next, a method of synthesizing the compound represented by General Formula (G1) will be described.

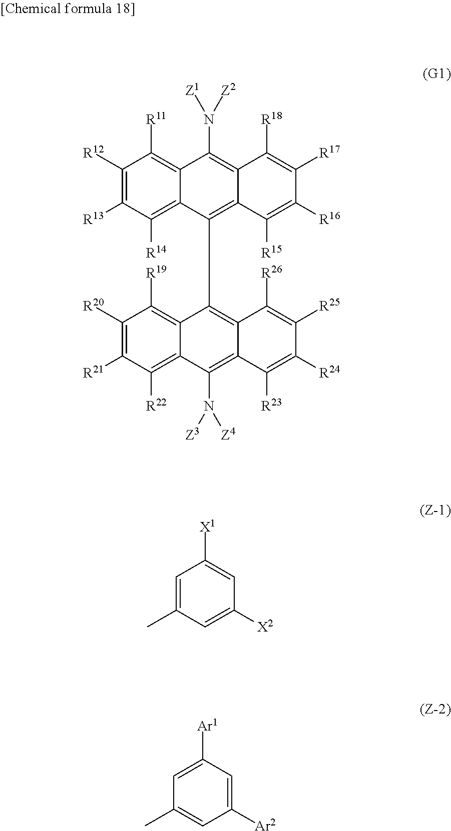

In General formula (G1), each of Z¹ to Z⁴ independently has a structure represented by General formula (Z-1) or General formula (Z-2). In General Formula (Z-1), each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of Ar¹ and Ar² independently represents an aromatic hydrocarbon group having 6 to 13 carbon atoms, and at least one of Ar¹ and Ar² includes a substituent that is the same as X¹. Moreover, each of R¹ to R²⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

The compound represented by General Formula (G1) above can be synthesized by, for example, Synthesis Scheme (S-1) and Synthesis Scheme (S-2) shown below.

First, a compound 1, a compound 2 (aniline compound), and a compound 3 (aniline compound) are coupled, whereby a compound 4 (diamine compound) can be obtained (Synthesis Scheme (S-1)).

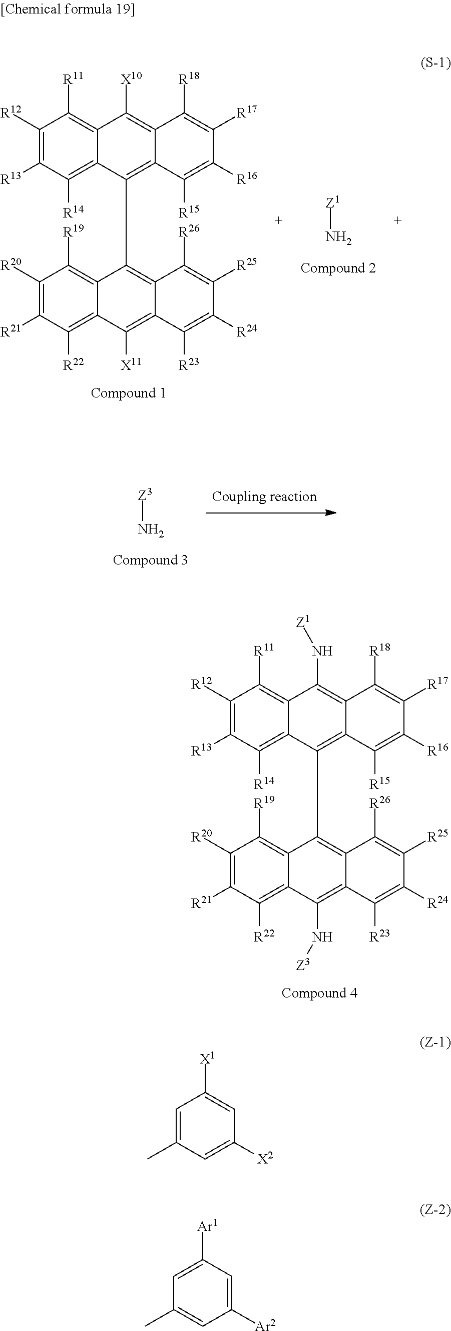

Next, the compound 4 (diamine compound), a compound 5 (halogenated aryl), and a compound 6 (halogenated aryl) are coupled, whereby the compound represented by General Formula (G1) can be obtained (Synthesis Scheme (S-2)).

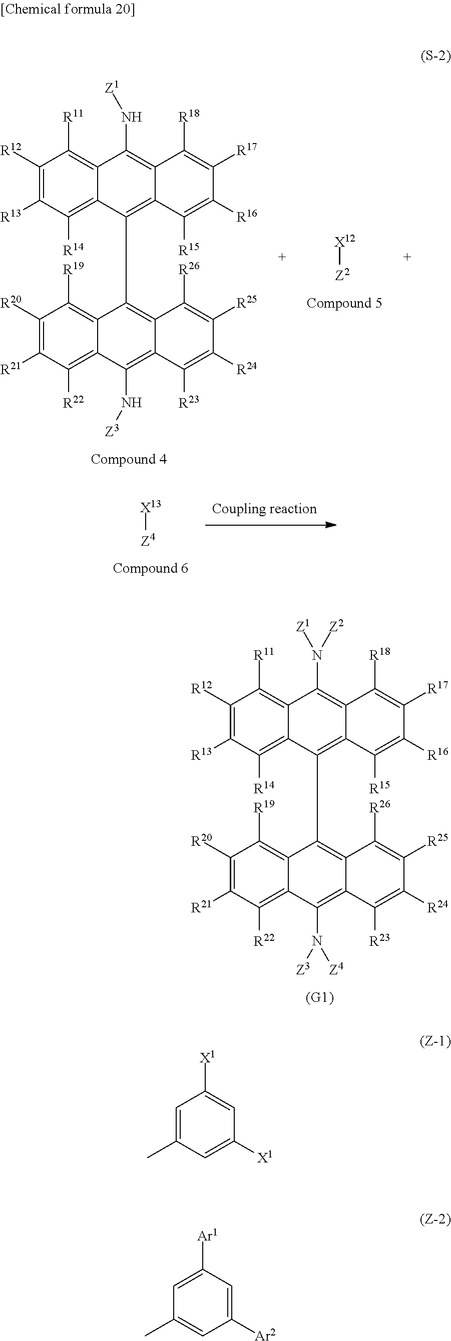

The compound represented by General Formula (G1) above can also be synthesized by methods shown in Synthesis Scheme (S-3), Synthesis Scheme (S-4), and Synthesis Scheme (S-5) below.

First, the compound 2 (aniline compound) and the compound 5 (halogenated aryl) are coupled, whereby a compound 7 (amine compound) can be obtained (Synthesis Scheme (S-3)).

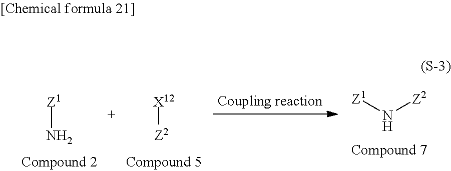

The compound 3 (aniline compound) and the compound 6 (halogenated aryl) are coupled, whereby a compound 8 (amine compound) can be obtained (Synthesis Scheme (S-4)).

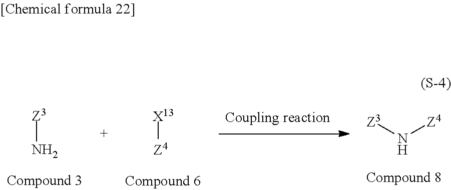

Next, the compound 1, the compound 7 (amine compound), and the compound 8 (amine compound) are coupled, whereby the compound represented by General Formula (G1) can be obtained (Synthesis Scheme (S-5)).

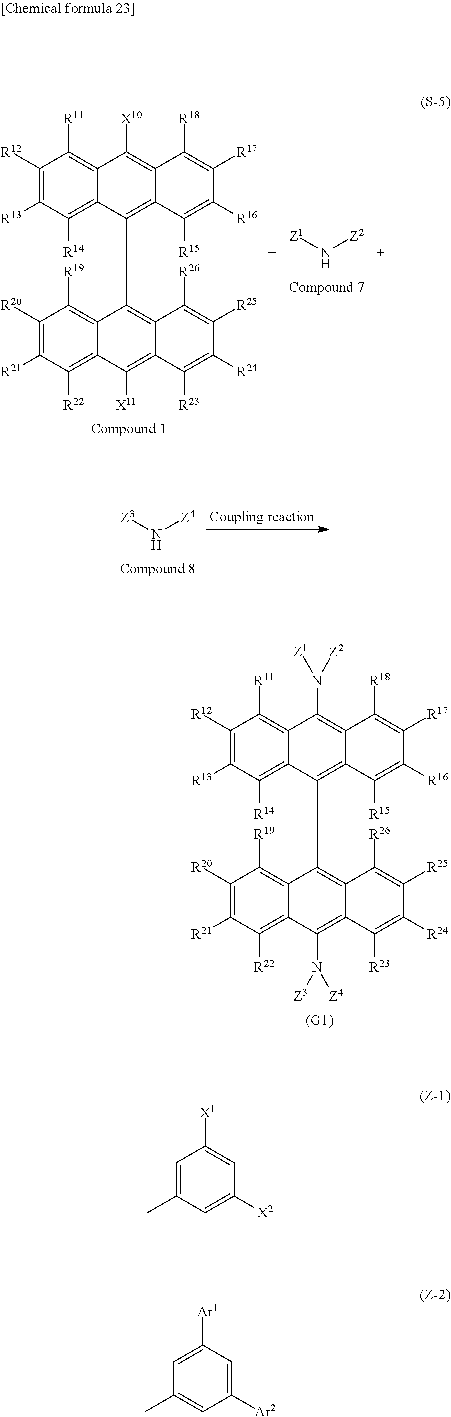

In Synthesis Schemes (S-1) to (S-5) above, each of Z¹ to Z⁴ independently has a structure represented by General Formula (Z-1) or General Formula (Z-2). In General Formula (Z-1), each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms. Moreover, each of Ar¹ and Ar² independently represents an aromatic hydrocarbon group having 6 to 13 carbon atoms, and at least one of Ar¹ and Ar² includes a substituent that is the same as X¹. Moreover, each of R¹ to R²⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

In the case where a Buchwald-Hartwig reaction using a palladium catalyst is performed in Synthesis Schemes (S-1) to (S-5) above, X¹⁰ to X¹³ each represent a halogen group or a triflate group, and the halogen is preferably iodine, bromine, or chlorine. In the reaction, a palladium compound such as bis(dibenzylideneacetone)palladium(0) or palladium(II) acetate and a ligand such as tri(tert-butyl)phosphine, tri(n-hexyl)phosphine, tricyclohexylphosphine, di(1-adamantyl)-n-butylphosphine, or 2-dicyclohexylphosphino-2′,6′-dimethoxy-1,1′-biphenyl can be used. In addition, an organic base such as sodium tert-butoxide, an inorganic base such as potassium carbonate, cesium carbonate, or sodium carbonate, or the like can be used. Furthermore, toluene, xylene, mesitylene, benzene, tetrahydrofuran, dioxane, or the like can be used as a solvent. Reagents that can be used in the reaction are not limited thereto.

The reaction performed in Synthesis Schemes (S-1) to (S-5) above is not limited to the Buchwald-Hartwig reaction. A Migita-Kosugi-Stille coupling reaction using an organotin compound, a coupling reaction using a Grignard reagent, an Ullmann reaction using copper or a copper compound, or the like can be used.

In the case where the compound 2 and the compound 3 have different structures in Synthesis Scheme (S-1) above, it is preferable that the compound 1 and the compound 2 be reacted to form a coupling product and then the obtained coupling product and the compound 3 be reacted. In the case where the compound 1 is reacted with the compound 2 and the compound 3 in different stages, it is preferable that the compound 1 be a dihalogen compound and X¹⁰ and X¹¹ be different halogens and selectively subjected to amination reactions one by one.

Furthermore, in Synthesis Scheme (S-2), it is preferable that the compound 4 and the compound 5 be reacted first to form a coupling product and then the obtained coupling product and the compound 6 be reacted.

Furthermore, in Synthesis Scheme (S-5), it is preferable that the compound 1 and the compound 7 be reacted first to form a coupling product and then the obtained coupling product and the compound 8 be reacted.

Although the method of synthesizing the compound of one embodiment of the present invention is described above, the present invention is not limited thereto and synthesis may be performed by any other synthesis method.

### Embodiment 2

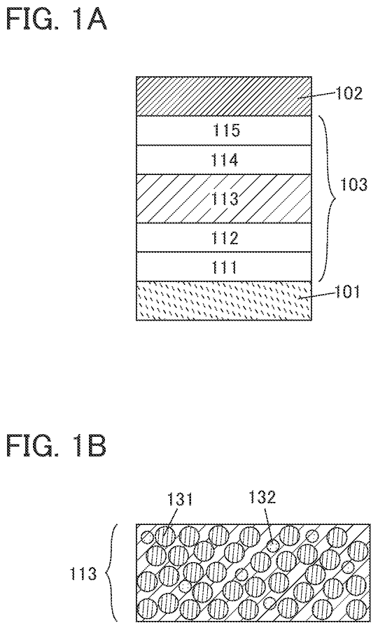

In this embodiment, an example of a light-emitting device, in which the compound of one embodiment of the present invention is preferably used, will be described. As shown in FIG. 1A, the light-emitting device has a structure in which an EL layer 103 is positioned between a pair of electrodes, a first electrode 101 (corresponding to an anode in FIG. 1A) and a second electrode 102 (corresponding to a cathode in FIG. 1A); the EL layer 103 includes at least a light-emitting layer 113; and furthermore, functional layers such as a hole-injection layer 111, a hole-transport layer 112, an electron-transport layer 114, and an electron-injection layer 115 can be provided.

The light-emitting layer 113 includes a light-emitting substance (guest material) and a host material. In the light-emitting device, voltage application between the pair of electrodes allows electrons and holes to be injected from the cathode and the anode, respectively, into the EL layer 103 and thus current flows. At this time, when carriers (electrons and holes) are recombined in the light-emitting layer 113, excitons are generated and excitation energy of the excitons is converted into light emission, whereby light emission can be obtained from the light-emitting device. Note that as illustrated in FIG. 1B, the light-emitting layer 113 in this embodiment contains a compound 132, which is an energy acceptor and functions as a light-emitting substance (guest material), and a compound 131, which is an energy donor and functions as a host material. Thus, in this embodiment, the case where the compound of one embodiment of the present invention is used as the light-emitting substance (guest material) is described. Note that the light-emitting layer 113 may include a plurality of compounds functioning as the host material.

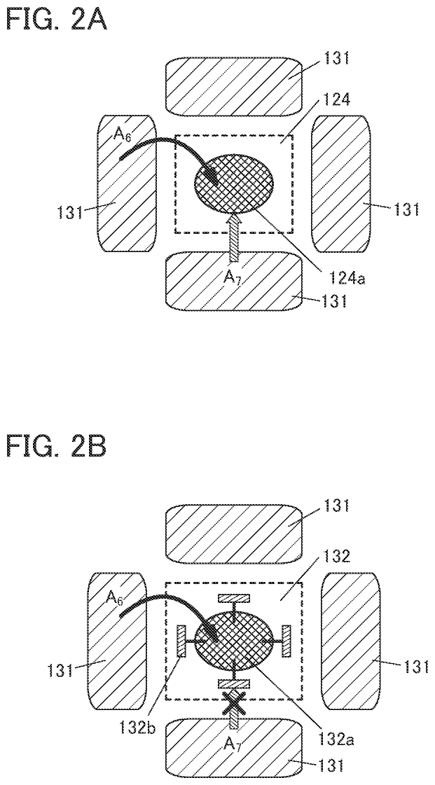

As to the excitons generated by the carrier recombination, the proportion of generation of singlet excitons is 25%, and the proportion of generation of triplet excitons is 75%; thus, it is preferable to make not only singlet excitons but also triplet excitons contribute to the light emission in order to improve the emission efficiency of the light-emitting device. Here, the concept of energy transfer that occurs between the guest material and the host material in the light-emitting layer 113 is described with reference to FIG. 2. Note that FIG. 2A illustrates a structure of a general guest material (fluorescent substance) and a concept of energy transfer between the guest material and the host material when the general guest material is used. FIG. 2B illustrates a structure of the compound 132 of one embodiment of the present invention and a concept of energy transfer between the guest material and the host material when the compound 132 is used as a guest material.

FIG. 2A shows a state where the compound 131 serving as a host material and a fluorescent substance 124 serving as a guest material are present. Note that the fluorescent substance 124 is a general fluorescent substance, and includes a luminophore 124a but does not include a protecting group.

FIG. 2B shows a state where the compound 131 serving as a host material and the compound (fluorescent substance) 132 of one embodiment of the present invention serving as a guest material are present. Note that the compound 132 is a fluorescent substance which is used as an energy acceptor in the light-emitting device, and includes a luminophore 132a and a protecting group 132b. Note that the protecting group 132b has a function of making the luminophore 132a away from the compound (host material) 131 at such a distance that energy transfer from the compound (host material) 131 to the luminophore 132a by the Dexter mechanism is less likely to occur.

In the light-emitting layer, the compound 131 serving as a host material is positioned close to the compound 124 and the compound (fluorescent substance) 132 serving as guest materials, as shown in FIG. 2A and FIG. 2B. In the case where the compound 124 does not include a protecting group as shown in FIG. 2A, the luminophore 124a and the compound 131 are close to each other; thus, both energy transfer by the Förster mechanism (Route A₆ in FIG. 2A) and energy transfer by the Dexter mechanism (Route A₇ in FIG. 2A) can occur as the energy transfer from the compound 131 to the compound 124. In the case where the guest material is a fluorescent substance, when the triplet excitation energy transfer from the host material to the guest material is caused by the Dexter mechanism and the triplet exited state of the guest material is generated, non-radiative deactivation of the triplet excitation energy occurs, contributing to a reduction in the emission efficiency of the light-emitting device.

In FIG. 2B, the compound (fluorescent substance) 132 serving as a guest material has the protecting group 132b, allowing the luminophore 132a and the compound 131 serving as a host material to be kept away from each other. This inhibits energy transfer by the Dexter mechanism (Route A₇).

Here, the luminophore 124a included in the compound 124 shown in FIG. 2A and the luminophore 132a included in the compound (fluorescent substance) 132 shown in FIG. 2B are described. The luminophore (124a or 132a) refers to an atomic group (skeleton) that causes light emission in a fluorescent substance. The luminophore (124a or 132a) generally has a π bond and preferably has an aromatic ring, further preferably a fused aromatic ring or a fused heteroaromatic ring. Since the luminophore (124a or 132a) has two or more identical skeletons, the transition dipole moment for light emission increases and hence the molar absorption coefficient increases, which is preferable because the rate of excitation energy transfer from the host material by the Förster mechanism can be increased. Examples of the fused aromatic ring or the fused heteroaromatic ring included in the luminophore (124a or 132a) include a phenanthrene ring, a stilbene ring, an acridone ring, a phenoxazine ring, and a phenothiazine ring. Specific examples are a naphthalene ring, an anthracene ring, a fluorene ring, a chrysene ring, a triphenylene ring, tetracene ring, a pyrene ring, a perylene ring, a quinoline ring, a benzimidazole ring, a quinazoline ring, a carbazole ring, an acridine ring, a coumalin ring, a quinacridone ring, a naphthobisbenzofuran ring, a dibenzofuran ring, a dibenzothiophene ring, an indenocarbazole ring, an indolocarbazole ring, and a dibenzo carbazole ring. The luminophore 132a included in the compound 132 of one embodiment of the present invention is preferably an anthracene skeleton, and particularly preferably has a bianthracene skeleton in which the 9-position of one of the two anthracene skeletons and the 10-position of the other are bonded.

The protecting group 132b in the compound (fluorescent substance) 132 shown in FIG. 2B preferably has a T1 level higher than the T1 levels of the luminophore 132a and the compound 131 serving as a host material. Note that specific examples of the protecting group 132b of the compound 132, which is one embodiment of the present invention, include an alkyl group having 3 to 10 carbon atoms, a substituted or unsubstituted cycloalkyl group having 3 to 10 carbon atoms, and a trialkylsilyl group having 3 to 12 carbon atoms. When the protective group 132b as described above is included, a bulky structure can be obtained; accordingly, the distance between the luminophore 132a of the compound 132 serving as a guest material and the compound 131 serving as a host material can be long.

Next, a structure of the light-emitting layer of the light-emitting device of one embodiment of the present invention will be described.

### Structure Example 1 of Light-Emitting Layer

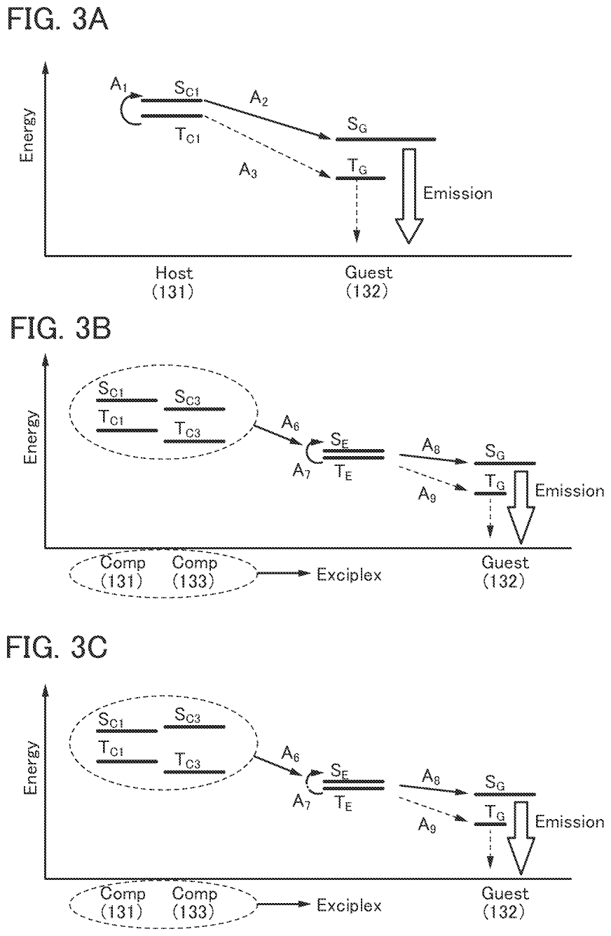

This structure example shows the light-emitting layer in the light-emitting device, which includes the compound 131 functioning as a host material and the compound 132 functioning as a light-emitting substance (guest material). A TADF material is used as the compound 131, and a fluorescent substance is used as the compound 132 serving as the light-emitting substance (guest material). Thus, it is preferable that the compound of one embodiment of the present invention be used as the compound 132 which is a fluorescent substance. FIG. 3A shows an example of the correlation of energy levels in the light-emitting layer 113 in this structure example. The following explains what the terms and numerals in FIG. 3A represent.
Host (131): the compound 131
Guest (132): the compound 132
T_{C1}: the T1 level of the compound 131
S_{C1}: the S1 level of the compound 131
S_{G}: the S1 level of the compound 132
T_{G}: the T1 level of the compound 132

In this structure example, since the compound 131 is a material with TADF, the compound 131 has a function of converting triplet excitation energy into singlet excitation energy by upconversion (Route A₁ in FIG. 3A). The singlet excitation energy of the compound 131 is rapidly transferred to the compound 132 (Route A₂ in FIG. 3A). At this time, the preferable relation between the S_{C1} of the compound 131 and the S_{G} of the compound 132 is S_{C1}≥S_{G}. Note that S_{C1} denotes energy with the wavelength of a line obtained by extrapolating a tangent to the fluorescent spectrum of the compound 131 at a tail on the short wavelength side. In addition, S_{G} denotes energy with the wavelength of the absorption edge of the absorption spectrum of the compound 132.

Thus, the triplet excitation energy generated in the compound 131 is transferred to the S1 level of the compound 132 serving as a guest material through Route A₁ and Route A₂, whereby efficient light emission (Emission) of the compound 132 is achieved to improve the emission efficiency of the light-emitting device. In Route A₂, the compound 131 functions as an energy donor and the compound 132 functions as an energy acceptor. Note that in the light-emitting layer 113 in the light-emitting device described in this structure example, the above routes might compete with a route through which the triplet excitation energy generated in the compound 131 is transferred to the T1 level of the compound 132 (Route A₃ in FIG. 3A). When such energy transfer (Route A₃) occurs, the compound 132 that is a fluorescent substance cannot make the triplet excitation energy contribute to light emission, which reduces the emission efficiency of the light-emitting device.

As mechanisms of the intermolecular energy transfer, the Förster mechanism (dipole-dipole interaction) and the Dexter mechanism (electron exchange interaction) are typically known. The Dexter mechanism is generated dominantly when the distance between the compound serving as an energy donor and the compound serving as an energy acceptor is 1 nm or shorter. Accordingly, when the compound 132 serving as an energy acceptor is a fluorescent material having a low triplet excitation energy level and the concentration of the compound 132 is high as in this structure example, as to the triplet excitation energy of the compound 131 serving as an energy donor, energy transfer by the Dexter mechanism through Route A₃ and non-radiative decay of the triplet excitation energy after the energy transfer are dominant. Therefore, in order to inhibit the energy transfer through Route A₃, it is important to make the distance between the compound 131 and the compound 132 long enough not to cause the energy transfer by the Dexter mechanism.

The T1 level (T_{G}) of the compound 132 serving as an energy acceptor is derived from the luminophore included in the compound 132 in many cases. Therefore, it is important to increase the distance between the compound 131 and the luminophore included in the compound 132 in order to inhibit energy transfer through Route A₃ in the light-emitting layer 113.

In general, as an example of a method of lengthening the distance between an energy donor and a luminophore included in an energy acceptor, lowering the concentration of the energy acceptor in the mixed film is given. However, lowering the concentration of the energy acceptor inhibits not only energy transfer based on the Dexter mechanism from the energy donor to the energy acceptor but also energy transfer based on the Förster mechanism from the energy donor to the energy acceptor. In that case, a problem such as a decrease in the emission efficiency and reliability of the light-emitting device is caused because Route A₂ is based on the Förster mechanism. The compound of one embodiment of the present invention includes a luminophore and a protective group in its structure. In the case where the compound of one embodiment of the present invention functions as the energy acceptor in the light-emitting layer 113, the protective group has a function of lengthening the distance between another energy donor and the luminophore. Thus, in the case where the compound of one embodiment of the present invention is used as the compound 132, the distance between the compound 132 and the compound 131 can be long. In general, the Dexter mechanism is dominant when the distance between the energy donor and the energy acceptor is shorter than or equal to 1 nm, and the Förster mechanism is dominant when the distance is longer than or equal to 1 nm and shorter than or equal to 10 nm. For this reason, the protective group is preferably a bulky substituent ranging from 1 nm to 10 nm from the luminophore. As the protective group included in the compound of one embodiment of the present invention, the above-described protective group is preferably used. With the use of the compound of one embodiment of the present invention as the compound 132, even when the concentration of the compound 132 is increased, the rate of energy transfer by the Förster mechanism can be increased while the energy transfer by the Dexter mechanism is inhibited. In other words, singlet excitation energy transfer (Route A₂) from the S1 level (S_{C1}) of the compound 131 to the S1 level (S_{G}) of the compound 132 is likely to occur while triplet excitation energy transfer (Route A₃: energy transfer by the Dexter mechanism) from the compound 131 to the T1 level (T_{G}) of the compound 132 can be less likely to occur. Thus, the emission efficiency of the light-emitting device can be increased while a decrease in emission efficiency due to energy transfer through Route A₃ can be inhibited. By increasing the rate of energy transfer due to the Förster mechanism, the excitation lifetime of the energy acceptor in the light-emitting layer is shortened, improving the reliability of the light-emitting device. Specifically, the concentration of the compound 132 in the light-emitting layer 113 is preferably greater than or equal to 2 wt % and less than or equal to 50 wt %, more preferably greater than or equal to 5 wt % and less than or equal to 30 wt %, further more preferably greater than or equal to 5 wt % and less than or equal to 20 wt % of the compound 131 serving as an energy donor.

### Structure Example 2 of Light-Emitting Layer

This structure example shows the case in which the light-emitting layer 113 in the light-emitting device includes the compound 131, the compound 132, and a compound 133, a combination of the compound 131 and the compound 133 forms an exciplex, and a fluorescent substance is used (ExEF is utilized) as the compound 132 functioning as the light-emitting substance (guest material). Thus, it is preferable that the compound of one embodiment of the present invention be used as the compound 132 which is a fluorescent substance. FIG. 3B shows an example of the correlation of energy levels in the light-emitting layer 113 in this structure example. The following explains what the terms and numerals in FIG. 3B represent.
Comp (131): the compound 131
Comp (133): the compound 133
Guest (132): the compound 132
S_{C1}: the S1 level of the compound 131
T_{C1}: the T1 level of the compound 131
S_{C3}: the S1 level of the compound 133
T_{C3}: the T1 level of the compound 133
S_{G}: the S1 level of the compound 132
T_{G}: the T1 level of the compound 132
S_{E}: the S1 level of the exciplex
T_{E}: the T1 level of the exciplex

Although any combination of the compound 131 and the compound 133 that can form an exciplex is acceptable, it is further preferable that one of them be a compound having a function of transporting holes (hole-transport property) and the other be a compound having a function of transporting electrons (electron-transport property). In this case, a donor-acceptor exciplex is easily formed; thus, efficient formation of an exciplex is possible. In the case where the combination of the compound 131 and the compound 133 is a combination of a compound having a hole-transport property and a compound having an electron-transport property, the carrier balance can be easily controlled by the mixture ratio. Specifically, the ratio of the compound having a hole-transport property to the compound having an electron-transport property is preferably within a range of 1:9 to 9:1 (weight ratio). Since the carrier balance can be easily controlled with the structure, a carrier recombination region can also be controlled easily.

For the combination of host materials for forming an exciplex efficiently, it is preferable that the HOMO level of one of the compound 131 and the compound 133 be higher than the HOMO level of the other and the LUMO level of the one of the compounds be higher than the LUMO level of the other. Note that the HOMO level of the compound 131 may be equivalent to the HOMO level of the compound 133, or the LUMO level of the compound 131 may be equivalent to the LUMO level of the compound 133.

Note that the LUMO levels and the HOMO levels of the compounds can be derived from the electrochemical characteristics (reduction potentials and oxidation potentials) of the compounds that are measured by cyclic voltammetry (CV) measurement.

As illustrated in FIG. 3B, the S1 level (S_{E}) and the T1 level (T_{E}) of the exciplex formed by the compound 131 and the compound 133 are energy levels adjacent to each other (see Route A₆ in FIG. 3B).

Because the excitation energy levels (S_{E} and T_{E}) of the exciplex are lower than the S1 levels (S_{C1} and S_{C3}) of the substances (the compound 131 and the compound 133) that form an exciplex, an excited state can be formed with lower excitation energy. Accordingly, the driving voltage of the light-emitting device can be reduced.

Since the S1 level (S_{E}) and the T1 level (T_{E}) of the exciplex are energy levels adjacent to each other, reverse intersystem crossing occurs easily, i.e., the exciplex has a TADF property. Therefore, the exciplex has a function of converting triplet excitation energy into singlet excitation energy by upconversion (Route A₇ in FIG. 3B). The singlet excitation energy of the exciplex can rapidly be transferred to the compound 132 (Route A₈ in FIG. 3B). At this time, S_{E}≥S_{G} is preferably satisfied. In Route A₈, the exciplex serves as an energy donor and the compound 132 serves as an energy acceptor. Specifically, when the level of energy with the wavelength of the line obtained by extrapolating a tangent to the fluorescent spectrum of the exciplex at a tail on the short wavelength side is S_{E} and the level of energy with the wavelength of the absorption edge of the absorption spectrum of the compound 132 is S_{G}, S_{E}≥S_{G} is preferably satisfied.

Note that in order to improve the TADF property, it is preferable that the T1 levels of both of the compound 131 and the compound 133, that is, T_{C1} and T_{C3} be higher than or equal to T_{E}. As the index for them, the emission peak wavelengths of the phosphorescent spectra of the compound 131 and the compound 133 on the shortest wavelength side are each preferably less than or equal to the maximum emission peak wavelength of the exciplex. When the level of energy with the wavelength of the line obtained by extrapolating a tangent to the fluorescent spectrum of the exciplex at a tail on the short wavelength side is S_{E} and the levels of energies with wavelengths of the lines obtained by extrapolating tangents to the phosphorescent spectra of the compound 131 and the compound 133 at a tail on the short wavelength side are T_{C1} and T_{C3}, respectively, S_{E}−T_{C1}≤0.2 eV and S_{E}−T_{C3}≤0.2 eV are preferably satisfied.

The triplet excitation energy generated in the light-emitting layer 113 is transferred to the S1 level of the compound 132 serving as a guest material through Route A₆ and Route A₈, whereby the compound 132 can emit light. Thus, the use of a combination of materials that form an exciplex in the light-emitting layer 113 can improve the emission efficiency of the fluorescent light-emitting device. However, the above routes might compete with a route through which the triplet excitation energy generated in the light-emitting layer 113 is transferred to the T1 level of the compound 132 (Route A₉ in FIG. 3B). When such energy transfer (Route A₉) occurs, the compound 132 that is a fluorescent substance cannot make the triplet excitation energy contribute to light emission, which reduces the emission efficiency of the light-emitting device.

In order to inhibit such energy transfer (Route A₉ in FIG. 3B), as described in Structure example 1 above, it is important that the distance between the compound 132 and the exciplex formed by the compound 131 and the compound 133 be long, and that the distance between the exciplex and the luminophore included in the compound 132 be long.

The compound of one embodiment of the present invention includes a luminophore and a protective group in its structure. In the case where the compound of one embodiment of the present invention functions as the energy acceptor in the light-emitting layer 113, the protective group has a function of lengthening the distance between another energy donor and the luminophore. Thus, in the case where the compound of one embodiment of the present invention is used as the compound 132 in this structure, the distance between the compound 132 and an exciplex formed by the compound 131 and the compound 133 can be long even when the concentration of the compound 132 is increased; accordingly, the rate of energy transfer by the Förster mechanism can be increased while energy transfer by the Dexter mechanism can be inhibited. With the use of the compound of one embodiment of the present invention as the compound 132, triplet excitation energy transfer (Route A₆ and Route A₈ in FIG. 3B) from the exciplex to the S1 level (S_{G}) of the compound 132 is likely to occur while triplet excitation energy transfer (Route A₉: energy transfer by the Dexter mechanism) from the exciplex to the T1 level (T_{G}) of the compound 132 can be less likely to occur. Thus, the emission efficiency of the light-emitting device can be increased while a decrease in emission efficiency due to energy transfer through Route A₉ can be inhibited. Thus, the emission efficiency of the light-emitting device can be increased while a decrease in emission efficiency due to energy transfer through Route A₉ can be inhibited. The reliability of the light-emitting device can be improved.

Note that in this specification, Route A₆, Route A₇, and Route A₈, which are described above, are also referred to as ExSET (Exciplex-Singlet Energy Transfer) or ExEF (Exciplex-Enhanced Fluorescence). In other words, in the light-emitting layer 113 in this specification, excitation energy is supplied from the exciplex to the fluorescent material.

### Structure Example 3 of Light-Emitting Layer

This structure example shows the case in which a fluorescent substance is used (ExEF is utilized) as the compound 132 functioning as the light-emitting substance (guest material), where the light-emitting layer 113 in the light-emitting device includes the compound 131, the compound 132, and a compound 133 and a combination of the compound 131 and the compound 133 forms an exciplex. In addition, this structure example is different from Structure example 2 in that the compound 133 is a phosphorescent material. It is preferable that the compound of one embodiment of the present invention be used as the compound 132 which is a fluorescent substance. FIG. 3C shows an example of the correlation of energy levels in the light-emitting layer 113 in this structure example. Note that terms and numerals in FIG. 3C are the same as those in FIG. 3B and thus the description thereof is omitted.

In this structure example, a compound containing a heavy atom is used as one of the compounds that form an exciplex. Thus, intersystem crossing between a singlet state and a triplet state is promoted. Thus, an exciplex capable of transition from a triplet excited state to a singlet ground state (i.e., capable of exhibiting phosphorescence) can be formed. In this case, unlike in the case of a typical exciplex, the triplet excitation energy level (T_{E}) of the exciplex is the level of an energy donor; thus, T_{E} is preferably higher than or equal to the singlet excitation energy level (S_{G}) of the compound 132, which is a light-emitting material. Specifically, T_{E} S_{G} is preferably satisfied when T_{E} is the level of energy with the wavelength of the line obtained by extrapolating a tangent to the emission spectrum of the exciplex using a heavy atom at a tail on the short wavelength side and S_{G} is the level of energy with the wavelength of the absorption edge of the absorption spectrum of the compound 132.

With such energy level correlation, the triplet excitation energy of the formed exciplex can be transferred from the triplet excitation energy level (T_{E}) of the exciplex to the singlet excitation energy level (S_{G}) of the compound 132. Note that it is sometimes difficult to clearly distinguish fluorescence and phosphorescence from each other in an emission spectrum because the S1 level (S_{E}) and the T1 level (T_{E}) of the exciplex are energy levels adjacent to each other. In that case, fluorescence and phosphorescence can be sometimes distinguished from each other by the emission lifetime.

The phosphorescent material used in the above structure preferably includes a heavy atom such as Jr, Pt, Os, Ru, or Pd. By contrast, the quantum yield can be either high or low because a phosphorescent material serves as an energy donor in this structure example. That is, energy transfer from the triplet excitation energy level of the exciplex to the singlet excitation energy level of the guest material is acceptable as long as it is allowable transition. The energy transfer from the phosphorescent material or the exciplex formed using a phosphorescent material to the guest material is a preferred structure, in which energy transfer from the triplet excitation energy level of the energy donor to the singlet excitation energy level of the guest material (energy acceptor) is allowable transition.

Thus, in the light-emitting layer 113 of the light-emitting device described in this structure example, the triplet excitation energy of the exciplex is transferred to the S1 level (S_{G}) of the guest material through Route A₈ (without passing through Route A₇ in FIG. 3C), as illustrated in FIG. 3C. That is, triplet excitation energy and singlet excitation energy can be transferred to the S1 level of the guest material only through Route A₆ and Route A₈. In Route A₈, the exciplex serves as an energy donor and the compound 132 functions as an energy acceptor. Note that in the light-emitting layer 113 in the light-emitting device described in this structure example, the above routes might compete with a route through which the triplet excitation energy exciplex is transferred to the T1 level of the compound 132 (Route A₉ in FIG. 3C). When such energy transfer (Route A₉) occurs, the compound 132 that is a fluorescent substance cannot make the triplet excitation energy contribute to light emission, which reduces the emission efficiency of the light-emitting device.

In order to inhibit such energy transfer (Route A₉), as described above in Structure example 1, it is important that the distance between the compound 131 and the compound 132 and the distance between the compound 131 and the luminophore included in the compound 132 be long.

The compound of one embodiment of the present invention includes a luminophore and a protective group in its structure. In the case where the compound of one embodiment of the present invention functions as the energy acceptor in the light-emitting layer 113, the protective group has a function of lengthening the distance between another energy donor and the luminophore. Thus, in the case where the compound of one embodiment of the present invention is used as the compound 132 in this structure, the distance between the compound 132 and an exciplex formed by the compound 131 and the compound 133 can be long even when the concentration of the compound 132 is increased; accordingly, the rate of energy transfer by the Förster mechanism can be increased while energy transfer by the Dexter mechanism can be inhibited. With the use of the compound of one embodiment of the present invention as the compound 132, triplet excitation energy transfer (Route A₆ and Route A₈) from the exciplex to the S1 level (S_{G}) of the compound 132 is likely to occur while triplet excitation energy transfer (Route A₉: energy transfer by the Dexter mechanism) from the exciplex to the T1 level (T_{G}) of the compound 132 can be less likely to occur. Thus, the emission efficiency of the light-emitting device can be increased while a decrease in emission efficiency due to energy transfer through Route A₉ can be inhibited. The reliability of the light-emitting device can be improved.

### Structure Example 4 of Light-Emitting Layer

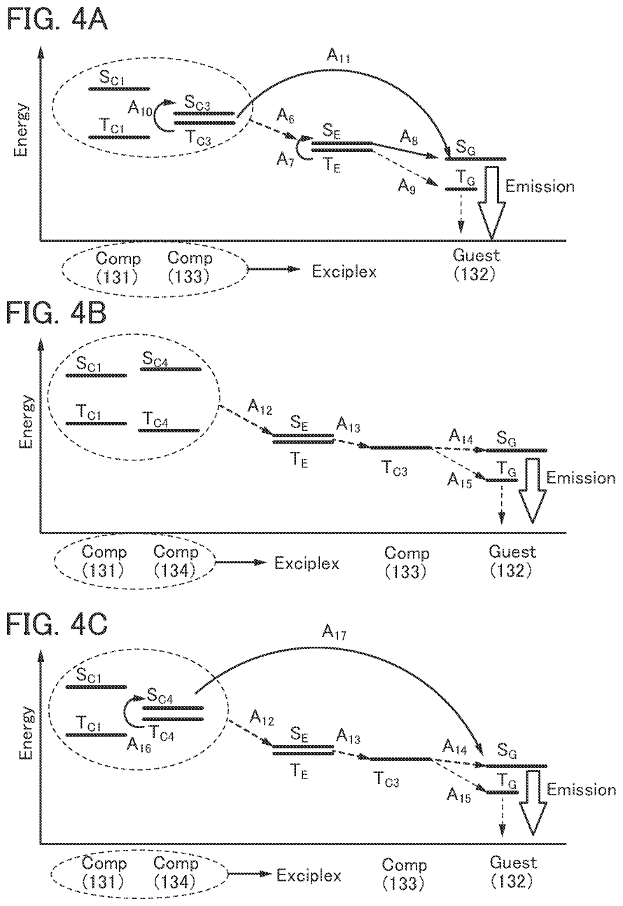

This structure example shows the light-emitting layer 113 in the light-emitting device, which includes three kinds of substances: the compound 131, the compound 132, and the compound 133. A combination of the compound 131 and the compound 133 forms an exciplex. The case in which a fluorescent substance is used (ExEF is utilized) as the compound 132 functioning as the light-emitting substance (guest material) is described. Thus, it is preferable that the compound of one embodiment of the present invention be used as the compound 132 which is a fluorescent substance. Note that this structure example is different from Structure example 3 in that the compound 133 is a material having a TADF property. FIG. 4A shows an example of the correlation of energy levels in the light-emitting layer 113 in this structure example. Note that terms and numerals in FIG. 4A are the same as those in FIG. 3B and thus the description thereof is omitted.

In this structure example, since the compound 133 is the TADF material, the compound 133 that does not form an exciplex has a function of converting triplet excitation energy into singlet excitation energy by upconversion (Route A₁₀ in FIG. 4A). Consequently, the singlet excitation energy of the compound 133 is rapidly transferred to the compound 132 (Route A₁₁ in FIG. 4A). At this time, S_{C3}≥S_{G} is preferably satisfied.

Thus, as described above in Structure example 3, the light-emitting layer 113 of the light-emitting device described in this structure example has a pathway where the triplet excitation energy is transferred to the compound 132 serving as a guest material through Route A₆ to Route A₈ in FIG. 4A and a pathway where the triplet excitation energy is transferred to the compound 132 through Route A₁₀ and Route A₁₁ in FIG. 4A. In this way, there are a plurality of paths through each of which the triplet excitation energy is transferred to the compound 132 that is a fluorescent substance, which can further increase the emission efficiency. In Route A₈, the exciplex functions as an energy donor and the compound 132 functions as an energy acceptor. In Route A₁₁, the compound 133 functions as an energy donor and the compound 132 functions as an energy acceptor. Note that in the light-emitting layer 113 in the light-emitting device described in this structure example, the above routes might compete with a route through which the triplet excitation energy of the exciplex is transferred to the T1 level of the compound 132 (Route A₉ in FIG. 4A). When such energy transfer (Route A₉) occurs, the compound 132 that is a fluorescent substance cannot make the triplet excitation energy contribute to light emission, which reduces the emission efficiency of the light-emitting device.

In order to inhibit such energy transfer (Route A₉), as described in Structure example 1 above, it is important that the distance between the compound 132 and the exciplex formed by the compound 131 and the compound 133 be long, that is, the distance between the exciplex formed by the compound 131 and the compound 133 and the luminophore included in the compound 132 be long.

The compound of one embodiment of the present invention includes a luminophore and a protective group in its structure. In the case where the compound of one embodiment of the present invention functions as the energy acceptor in the light-emitting layer 113, the protective group has a function of lengthening the distance between another energy donor and the luminophore. Thus, in the case where the compound of one embodiment of the present invention is used as the compound 132 in this structure, the distance between the compound 132 and an exciplex formed by the compound 131 and the compound 133 can be long even when the concentration of the compound 132 is increased; accordingly, the rate of energy transfer by the Förster mechanism can be increased while energy transfer by the Dexter mechanism can be inhibited. With the use of the compound of one embodiment of the present invention as the compound 132, both triplet excitation energy transfer (Route A₆ and Route A₈) from the exciplex to the S1 level (S_{G}) of the compound 132 and triplet excitation energy transfer (Route A₁₀ and Route A₁₁) from the exciplex to the S1 level (S_{G}) of the compound 132 are likely to occur while triplet excitation energy transfer (Route A₉: energy transfer by the Dexter mechanism) from the exciplex to the T1 level (T_{G}) of the compound 132 can be less likely to occur. Thus, the emission efficiency of the light-emitting device can be increased while a decrease in emission efficiency due to energy transfer through Route A₉ can be inhibited. The reliability of the light-emitting device can be improved.

### Structure Example 5 of Light-Emitting Device

This structure example shows the light-emitting layer 113 in the light-emitting device, which includes four kinds of substances: the compound 131, the compound 132, the compound 133, and the compound 134. Note that the compound 133 has a function of being capable of converting triplet excitation energy into light emission and is particularly a phosphorescent substance. A combination of the compound 131 and the compound 134 forms an exciplex. The case in which a fluorescent substance is used as the compound 132 functioning as the light-emitting substance (guest material) is described. Thus, it is preferable that the compound of one embodiment of the present invention be used as the compound 132 which is a fluorescent substance. FIG. 4B shows an example of the correlation of energy levels in the light-emitting layer 113 in this structure example. Note that terms and numerals in FIG. 4B are similar to those in FIG. 3B and the other terms and numerals are as follows:
S_{C4}: the S1 level of the compound 134
T_{C4}: the T1 level of the compound 134

In this structure example, the compound 131 and the compound 134 form an exciplex. The S1 level (S_{E}) of the exciplex and the T1 level (T_{E}) of the exciplex are energy levels adjacent to each other (see Route A₁₂ in FIG. 4B). Note that when the exciplex formed by the two kinds of substances through the above path loses excitation energy, the two kinds of substances exist as the original different substances.

Because the excitation energy levels (S_{E} and T_{E}) of the exciplex are lower than the S1 levels (S_{C1} and S_{C4}) of the substances (the compound 131 and the compound 134) that form an exciplex, an excited state can be formed with lower excitation energy. Accordingly, the driving voltages can be reduced.

When the compound 133 is a phosphorescent material, intersystem crossing between a singlet state and a triplet state is allowed. Hence, both the singlet excitation energy and the triplet excitation energy are rapidly transferred from the exciplex to the compound 133 (Route A₁₃). At this time, T_{E}≥T_{C3} is preferably satisfied.

The triplet excitation energy of the compound 133 is converted into the singlet excitation energy of the compound 132 (Route A₁₄). At this time, it is preferable that the relation T_{E}≥T_{C3}≥S_{G} be satisfied as shown in FIG. 4B because energy is transferred efficiently from the compound 133 to the compound 132. Specifically, T_{C3}≥S_{G} is preferably satisfied when T_{C3} is energy with the wavelength of the line obtained by extrapolating a tangent to the phosphorescent spectrum of the compound 133 at a tail on the short wavelength side, and S_{G} is energy with a wavelength of the absorption edge of the absorption spectrum of the compound 132. In Route A₁₄, the compound 133 serves as an energy donor and the compound 132 serves as an energy acceptor.

In this structure example, although any combination of the compound 131 and the compound 134 that can form an exciplex is acceptable, it is further preferable that one of them be a compound having a hole-transport property and the other be a compound having an electron-transport property.

For the combination of materials for forming an exciplex efficiently, it is preferable that the HOMO level of one of the compound 131 and the compound 134 be higher than the HOMO level of the other and the LUMO level of the one of the compounds be higher than the LUMO level of the other.

The correlation between the energy levels of the compound 131 and the compound 134 is not limited to that in FIG. 4B. In other words, the singlet excitation energy level (S_{C1}) of the compound 131 may be higher or lower than the singlet excitation energy level (S_{C4}) of the compound 134. The triplet excitation energy level (T_{C1}) of the compound 131 may be higher or lower than the triplet excitation energy level (T_{C4}) of the compound 134.

In the light-emitting device of this structure example, the compound 131 preferably has a π-electron deficient skeleton. Such a composition lowers the LUMO level of the compound 131, which is suitable for formation of an exciplex.

In the light-emitting device of this structure example, the compound 131 preferably has a π-electron rich skeleton. Such a composition increases the HOMO level of the compound 131, which is suitable for formation of an exciplex.

The compound of one embodiment of the present invention includes a luminophore and a protective group in its structure. In the case where the compound of one embodiment of the present invention functions as the energy acceptor in the light-emitting layer 113, the protective group has a function of lengthening the distance between another energy donor and the luminophore. Thus, in the case where the compound of one embodiment of the present invention is used as the compound 132, the distance between the compound 133 and the compound 132 can be long. With the use of the compound of one embodiment of the present invention as the compound 132, triplet excitation energy transfer (Route A₁₄) from the compound 133 to the S1 level (S_{G}) of the compound 132 is likely to occur while triplet excitation energy transfer (Route A₁₅: energy transfer by the Dexter mechanism) from the exciplex to the T1 level (T_{G}) of the compound 132 can be less likely to occur. Thus, the emission efficiency of the light-emitting device can be increased while a decrease in emission efficiency due to energy transfer through Route A₁₅ can be inhibited.

In this structure example, by increasing the concentration of the compound 132 serving as an energy acceptor, the rate of energy transfer by the Förster mechanism can be increased while the energy transfer by the Dexter mechanism is inhibited. By increasing the rate of energy transfer due to the Förster mechanism, the excitation lifetime of the energy acceptor in the light-emitting layer is shortened, improving the reliability of the light-emitting device. Specifically, the concentration of the compound 132 in the light-emitting layer 113 is preferably greater than or equal to 2 wt % and less than or equal to 50 wt %, more preferably greater than or equal to 5 wt % and less than or equal to 30 wt %, further more preferably greater than or equal to 5 wt % and less than or equal to 20 wt % of the compound 133 serving as an energy donor.

Note that in this specification, Route A₁₂ and Route A₁₃, which are the routes described above, are also referred to as ExTET (Exciplex-Triplet Energy Transfer). This indicates that, in the light-emitting layer 113 in this specification, excitation energy is supplied from the exciplex to the compound 133.

### Structure Example 6 of Light-Emitting Device

This structure example shows the light-emitting layer 113 in the light-emitting device, which includes four kinds of substances: the compound 131, the compound 132, the compound 133, and the compound 134. Note that the compound 133 has a function of being capable of converting triplet excitation energy into light emission and is particularly a phosphorescent substance. A combination of the compound 131 and the compound 134 forms an exciplex. The case in which a fluorescent substance is used as the compound 132 functioning as the light-emitting substance (guest material) is described. Thus, it is preferable that the compound of one embodiment of the present invention be used as the compound 132 which is a fluorescent substance. Note that this structure example is different from Structure example 5 in that the compound 134 is a material having a TADF property. FIG. 4C shows an example of the correlation of energy levels in the light-emitting layer 113 in this structure example. Note that terms and numerals in FIG. 4C are the same as those in FIG. 3B and FIG. 4B and thus the description thereof is omitted.

Here, since the compound 134 is the TADF material, the compound 134 that does not form an exciplex has a function of converting triplet excitation energy into singlet excitation energy by upconversion (Route A₁₆ in FIG. 4C). Thus, the singlet excitation energy of the compound 134 is rapidly transferred to the compound 132 (Route A₁₇ in FIG. 4C). At this time, S_{C4}≥S_{G} is preferably satisfied. Specifically, when the level of energy with the wavelength of the line obtained by extrapolating a tangent to the fluorescent spectrum of the compound 134 at a tail on the short wavelength side is S_{C4} and the level of energy with the wavelength of the absorption edge of the absorption spectrum of the compound 132 is S_{G}, S_{C4}≥S_{G} is preferably satisfied.

Thus, as described above in Structure example 5, the light-emitting layer 113 of the light-emitting device described in this structure example has a pathway where the triplet excitation energy is transferred to the compound 132 serving as a guest material through Route A₁₂, Route A₁₃, and Route A₁₄ in FIG. 4C and a pathway where the triplet excitation energy is transferred to the compound 132 through Route A₁₆ and Route A₁₇ in FIG. 4C. In this way, there are a plurality of paths through each of which the triplet excitation energy is transferred to the compound 132 that is a fluorescent substance, which can further increase the emission efficiency. In Route A₁₄, the compound 133 functions as an energy donor, and the compound 132 functions as an energy acceptor. In Route A₁₇, the compound 134 functions as an energy donor and the compound 132 functions as an energy acceptor. Note that in the light-emitting layer 113 in the light-emitting device described in this structure example, the above routes might compete with a route through which the triplet excitation energy of the compound 133 is transferred to the T1 level of the compound 132 (Route A₁₅ in FIG. 4C). When such energy transfer (Route A₁₅) occurs, the compound 132 that is a fluorescent substance cannot make the triplet excitation energy contribute to light emission, which reduces the emission efficiency of the light-emitting device.

In order to inhibit such energy transfer (Route A₁₅), as described above in Structure example 1, it is important that the distance between the compound 133 and the compound 132 and the distance between the compound 133 and the luminophore included in the compound 132 be long.

The compound of one embodiment of the present invention includes a luminophore and a protective group in its structure. In the case where the compound of one embodiment of the present invention functions as the energy acceptor in the light-emitting layer 113, the protective group has a function of lengthening the distance between another energy donor and the luminophore. Thus, in the case where the compound of one embodiment of the present invention is used as the compound 132 in this structure, the distance between the compound 133 and the compound 132 can be long even when the concentration of the compound 132 is increased; accordingly, the rate of energy transfer by the Förster mechanism can be increased while energy transfer by the Dexter mechanism can be inhibited. With the use of the compound of one embodiment of the present invention as the compound 132, both triplet excitation energy transfer (Route A₁₂, Route A₁₃, and Route A₁₄) from the exciplex to the S1 level (S_{G}) of the compound 132 and triplet excitation energy transfer ((Route A₁₆ and Route A₁₇) from the compound 133 to the S1 level (S_{G}) of the compound 132 are likely to occur while triplet excitation energy transfer (Route A₁₅: energy transfer by the Dexter mechanism) from the exciplex to the T1 level (T_{G}) of the compound 132 can be less likely to occur. Thus, the emission efficiency of the light-emitting device can be increased while a decrease in emission efficiency due to energy transfer through Route A₁₅ can be inhibited. The reliability of the light-emitting device can be improved.

### Structure Example 7 of Light-Emitting Layer

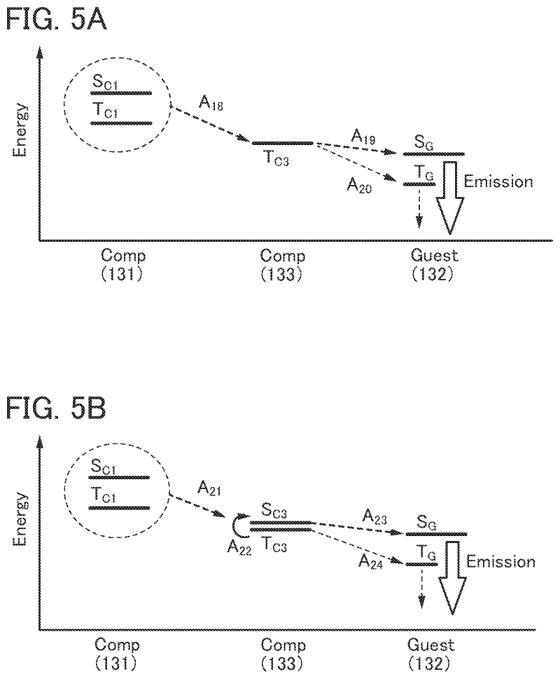

This structure example shows the light-emitting layer 113 in the light-emitting device, which includes three kinds of substances: the compound 131, the compound 132, and the compound 133. The compound 133 has a function of being capable of converting triplet excitation energy into light emission and is particularly a phosphorescent substance. A fluorescent substance is used as the compound 132 functioning as the light-emitting substance (guest material). Thus, it is preferable that the compound of one embodiment of the present invention be used as the compound 132 which is a fluorescent substance. FIG. 5A shows an example of the correlation of energy levels in the light-emitting layer 113 in this structure example. The following explains what the terms and numerals in FIG. 5A represent.
Comp (131): the compound 131
Comp (133): the compound 133
Guest (132): the compound 132
S_{C1}: the S1 level of the compound 131
T_{C1}: the T1 level of the compound 131
T_{C3}: the T1 level of the compound 133
T_{G}: the T1 level of the compound 132
S_{G}: the S1 level of the compound 132

In this structure example, carrier recombination occurs mainly in the compound 131, whereby singlet excitons and triplet excitons are generated. When a phosphorescent substance having a relation T_{C3}≤T_{C1} is selected as the compound 133, both the singlet excitation energy and triplet excitation energy generated in the compound 131 can be transferred to the T_{C3} level of the compound 133 (Route A₁₈ in FIG. 5A). Some of the carriers can be recombined also in the compound 133.

The phosphorescent substance used in the above structure preferably includes a heavy atom such as Jr, Pt, Os, Ru, or Pd. A phosphorescent substance is preferably used as the compound 133, in which case energy transfer from the triplet excitation energy level of the energy donor to the singlet excitation energy level of the guest material (energy acceptor) is allowable transition. Thus, the triplet excitation energy of the compound 133 can be transferred to the S1 level (S_{G}) of the guest material through the path of Route A₁₉. In Route A₁₉, the compound 133 serves as an energy donor and the compound 132 serves as an energy acceptor. In that case, T_{C3}≥S_{G} is preferably satisfied because the excitation energy of the compound 133 is efficiently transferred to the singlet excited state of the compound 132 serving as a guest material. Specifically, T_{C3}≥S_{G} is preferably satisfied when T_{C3} is energy with a wavelength of the line obtained by extrapolating a tangent to the phosphorescent spectrum of the compound 133 at a tail on the short wavelength side, and S_{G} is energy with a wavelength of the absorption edge of the absorption spectrum of the compound 132. Note that in the light-emitting layer 113 in the light-emitting device of this structure example, the above routes might compete with a route through which the triplet excitation energy of the compound 133 is transferred to the T1 level of the compound 132 (Route A₂₀ in FIG. 5A). When such energy transfer (Route A₂₀) occurs, the compound 132 that is a fluorescent substance cannot make the triplet excitation energy contribute to light emission, which reduces the emission efficiency of the light-emitting device.

In order to inhibit such energy transfer (Route A₂₀), as described above in Structure example 1, it is important that the distance between the compound 133 and the compound 132 and the distance between the compound 133 and the luminophore included in the compound 132 be long.

The compound of one embodiment of the present invention includes a luminophore and a protective group in its structure. In the case where the compound of one embodiment of the present invention functions as the energy acceptor in the light-emitting layer 113, the protective group has a function of lengthening the distance between another energy donor and the luminophore. Thus, in the case where the compound of one embodiment of the present invention is used as the compound 132 in this structure, the distance between the compound 133 and the compound 132 can be long even when the concentration of the compound 132 is increased; accordingly, the rate of energy transfer by the Förster mechanism can be increased while energy transfer by the Dexter mechanism can be inhibited. With the use of the compound of one embodiment of the present invention as the compound 132, triplet excitation energy transfer (Route A₁₉) from the compound 133 to the S1 level (S_{G}) of the compound 132 is likely to occur while triplet excitation energy transfer (Route A₂₀: energy transfer by the Dexter mechanism) from the exciplex to the T1 level (T_{G}) of the compound 132 can be less likely to occur. Thus, the emission efficiency of the light-emitting device can be increased while a decrease in emission efficiency due to energy transfer through Route A₂₀ can be inhibited. The reliability of the light-emitting device can be improved.

### Structure Example 8 of Light-Emitting Layer

This structure example shows the light-emitting layer 113 in the light-emitting device, which includes three kinds of substances: the compound 131, the compound 132, and the compound 133. Note that the compound 133 has a function of being capable of converting triplet excitation energy into light emission and is particularly a material having a TADF property. A fluorescent substance is used as the compound 132 functioning as the light-emitting substance (guest material). Thus, it is preferable that the compound of one embodiment of the present invention be used as the compound 132 which is a fluorescent substance. FIG. 5B shows an example of the correlation of energy levels in the light-emitting layer 113 in this structure example. Note that terms and numerals in FIG. 5B are similar to those in FIG. 5A and the other terms and numerals are as follows:
S_{C3}: the S1 level of the compound 133

In this structure example, carrier recombination occurs mainly in the compound 131, whereby singlet excitons and triplet excitons are generated. When a material having a TADF property having a relation S_{C3}≤S_{C1} and T_{C3}≤T_{C1} is selected as the compound 133, both the singlet excitation energy and triplet excitation energy generated in the compound 131 can be transferred to the S_{C3} and T_{C3} levels of the compound 133 (Route A₂₁ in FIG. 5B). Some of the carriers can be recombined also in the compound 133.

The compound 133 is a material having a TADF property and thus has a function of converting triplet excitation energy into singlet excitation energy by upconversion (Route A₂₂ in FIG. 5B). The singlet excitation energy of the compound 133 can be rapidly transferred to the compound 132 (Route A₂₃ in FIG. 5B). At this time, S_{C3}≥S_{G} is preferably satisfied. Specifically, when the level of energy with the wavelength of the line obtained by extrapolating a tangent to the fluorescent spectrum of the compound 133 at a tail on the short wavelength side is S_{C3} and the level of energy with the wavelength of the absorption edge of the absorption spectrum of the compound 132 is S_{G}, S_{C3}≥S_{G} is preferably satisfied.

Thus, in the light-emitting layer 113 of the light-emitting device described in this structure example, the triplet excitation energy generated in the compound 133 can be converted into fluorescence of the compound 132 by passing through Route A₂₁, Route A₂₂, and Route A₂₃ in FIG. 5B. In Route A₂₃, the compound 133 functions as an energy donor and the compound 132 functions as an energy acceptor. Note that in the light-emitting layer 113 in the light-emitting device of this structure example, the above routes might compete with a route through which the triplet excitation energy of the compound 133 is transferred to the T1 level of the compound 132 (Route A₂₄ in FIG. 5B). When such energy transfer (Route A₂₄) occurs, the compound 132 that is a fluorescent substance cannot make the triplet excitation energy contribute to light emission, which reduces the emission efficiency of the light-emitting device.

In order to inhibit such energy transfer (Route A₂₄), as described above in Structure example 1, it is important that the distance between the compound 133 and the compound 132 and the distance between the compound 133 and the luminophore included in the compound 132 be long.

The compound of one embodiment of the present invention includes a luminophore and a protective group in its structure. In the case where the compound of one embodiment of the present invention functions as the energy acceptor in the light-emitting layer 113, the protective group has a function of lengthening the distance between another energy donor and the luminophore. Thus, in the case where the compound of one embodiment of the present invention is used as the compound 132 in this structure, the distance between the compound 133 and the compound 132 can be long even when the concentration of the compound 132 is increased; accordingly, the rate of energy transfer by the Förster mechanism can be increased while energy transfer by the Dexter mechanism can be inhibited. With the use of the compound of one embodiment of the present invention as the compound 132, triplet excitation energy transfer (Route A₂₃) from the compound 133 to the S1 level (S_{G}) of the compound 132 is likely to occur while triplet excitation energy transfer (Route A₂₄: energy transfer by the Dexter mechanism) from the compound 133 to the T1 level (T_{G}) of the compound 132 can be less likely to occur. Thus, the emission efficiency of the light-emitting device can be increased while a decrease in emission efficiency due to energy transfer through Route A₂₄ can be inhibited. The reliability of the light-emitting device can be improved.

### Embodiment 3

In this embodiment, a light-emitting device of one embodiment of the present invention will be described.

### Structure Example of Light-Emitting Device

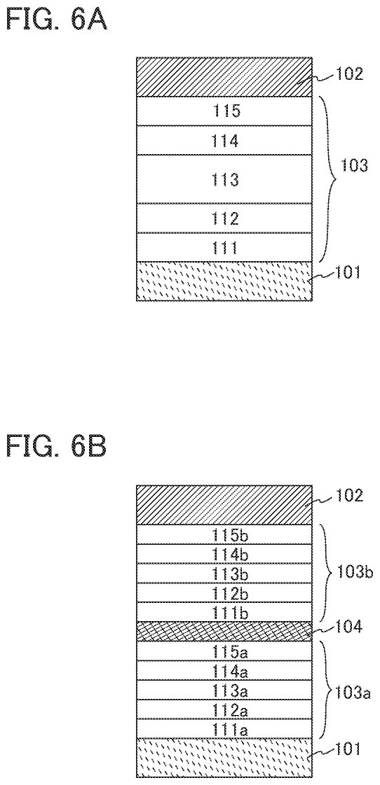

FIG. 6A shows an example of a light-emitting device including, between a pair of electrodes, an EL layer including a light-emitting layer. Specifically, the light-emitting device has a structure in which the EL layer 103 is sandwiched between the first electrode 101 and the second electrode 102. For example, in the case where the first electrode 101 serves as an anode, the EL layer 103 has a structure in which the hole-injection layer 111, the hole-transport layer 112, the light-emitting layer 113, the electron-transport layer 114, and the electron-injection layer 115 are sequentially stacked as functional layers. The light-emitting layer 113 includes a host material and a guest material; the third organic compound is used as the host material; and as the guest material, the first organic compound, which is a material (a fluorescent substance) having a function of converting singlet excitation energy into light emission, and the second organic compound, which is a material (a phosphorescent substance or a TADF material) having a function of converting triplet excitation energy into light emission, are used.

Embodiments of the present invention also include light-emitting devices having other structures, for example, a light-emitting device that can be driven at a low voltage by having a structure in which a plurality of EL layers, between which a charge-generation layer is sandwiched, are provided between a pair of electrodes (a tandem structure), and a light-emitting device that has improved optical characteristics by having a micro-optical resonator (microcavity) structure between a pair of electrodes. Note that the charge-generation layer has a function of injecting electrons into one of the adjacent EL layers and injecting holes into the other of the EL layers when a voltage is applied to the first electrode 101 and the second electrode 102.

Note that at least one of the first electrode 101 and the second electrode 102 of the above light-emitting device is an electrode having a light-transmitting property (e.g., a transparent electrode or a transflective electrode). In the case where the electrode having a light-transmitting property is a transparent electrode, the visible light transmittance of the transparent electrode is 40% or higher. In the case where the electrode having a light-transmitting property is a transflective electrode, the visible light reflectance of the transflective electrode is higher than or equal to 20% and lower than or equal to 80%, preferably higher than or equal to 40% and lower than or equal to 70%. The resistivity of these electrodes is preferably 1×10⁻² Ωcm or lower.

In the case where one of the first electrode 101 and the second electrode 102 is an electrode having reflectivity (a reflective electrode) in the above light-emitting device of one embodiment of the present invention, the visible light reflectance of the electrode having reflectivity is higher than or equal to 40% and lower than or equal to 100%, preferably higher than or equal to 70% and lower than or equal to 100%. The resistivity of this electrode is preferably 1×10⁻² Ωcm or lower.

### <First Electrode and Second Electrode>

As materials for forming the first electrode 101 and the second electrode 102, any of the following materials can be used in an appropriate combination as long as the functions of the electrodes described above can be fulfilled. For example, a metal, an alloy, an electrically conductive compound, and a mixture of these can be used as appropriate. Specific examples include In—Sn oxide (also referred to as ITO), In—Si—Sn oxide (also referred to as ITSO), In—Zn oxide, and In—W—Zn oxide. It is also possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use an element belonging to Group 1 or Group 2 in the periodic table, which is not listed above as an example (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

For fabrication of these electrodes, a sputtering method or a vacuum evaporation method can be used.

### <Hole-Injection Layer>

The hole-injection layer 111 is a layer injecting holes from the first electrode 101 that is an anode to the EL layer 103, and is a layer containing an organic acceptor material or a material with a high hole-injection property.

The organic acceptor material is a material that allows holes to be generated in another organic compound whose HOMO level value is close to the LUMO level value of the organic acceptor material when charge separation is caused between the organic acceptor material and the organic compound. Thus, as the organic acceptor material, a compound having an electron-withdrawing group (a halogen group or a cyano group), such as a quinodimethane derivative, a chloranil derivative, or a hexaazatriphenylene derivative, can be used. For example, 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), or 1,3,4,5,7,8-hexafluorotetracyano-naphthoquinodimethane (abbreviation: F6-TCNNQ) can be used. Among organic acceptor materials, HAT-CN, which has a high acceptor property and stable film quality against heat, is particularly favorable. Besides, a [3]radialene derivative has a very high electron-accepting property and thus is preferable; specifically, α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], α,α′,α″-1,2,3-cyclopropanetriylidenetris[2,3,4,5,6-pentafluorobenzeneacetonitrile], or the like can be used.

Examples of the material with a high hole-injection property include transition metal oxides such as molybdenum oxide, vanadium oxide, ruthenium oxide, tungsten oxide, and manganese oxide. It is also possible to use a phthalocyanine-based compound such as phthalocyanine (abbreviation: H₂Pc) or copper phthalocyanine (abbreviation: CuPc), or the like.

In addition to the above materials, it is also possible to use an aromatic amine compound, which is a low molecular compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-Y-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), or 3-[N-(1-naphthyl)-N-(9-phenylcarbazol-3-yl)amino]-9-phenylcarbazole (abbreviation: PCzPCN1).

It is also possible to use a high molecular compound (an oligomer, a dendrimer, a polymer, or the like) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N-phenylamino}phenyl)methacrylamide] (abbreviation: PTPDMA), or poly[N,N′-bis(4-butylphenyl)-N,N′-bis(phenyl)benzidine] (abbreviation: Poly-TPD). Alternatively, it is also possible to use a high molecular compound to which acid is added, such as poly(3,4-ethylenedioxythiophene)/poly(styrenesulfonic acid) (abbreviation: PEDOT/PSS) or polyaniline/poly(styrenesulfonic acid) (PAni/PSS).

Alternatively, as the material having a high hole-injection property, a composite material containing a hole-transport material and an acceptor material (electron-accepting material) can be used. In this case, the acceptor material extracts electrons from a hole-transport material, so that holes are generated in the hole-injection layer 111 and the holes are injected into the light-emitting layer 113 through the hole-transport layer 112. Note that the hole-injection layer 111 may be formed as a single layer made of a composite material containing a hole-transport material and an acceptor material (electron-accepting material), or may be formed by stacking a layer containing a hole-transport material and a layer containing an acceptor material (electron-accepting material).

As the hole-transport material, a substance having a hole mobility higher than or equal to 1×10⁻⁶ cm²/Vs is preferable. Note that other substances can be used as long as they have a property of transporting more holes than electrons.

As the hole-transport material, materials having a high hole-transport property, such as a π-electron rich heteroaromatic compound (e.g., a carbazole derivative and a furan derivative) and an aromatic amine (a compound having an aromatic amine skeleton), are preferable.

Examples of the above carbazole derivative (a compound having a carbazole skeleton) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(1,1′-biphenyl-4-yl)-3,3′-bi-9H-carbazole, 9,9′-bis(1,1′-biphenyl-3-yl)-3,3′-bi-9H-carbazole, 9-(1,1′-biphenyl-3-yl)-9′-(1,1′-biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: βNCCP).

Specific examples of the above aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-NX-diphenylbenzene-1,3-diamine (abbreviation: PCA2B), N,N′,N″-triphenyl-N,N′,″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamine]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

In addition to the above, other examples of the carbazole derivative include 3-[4-(9-phenanthryl)-phenyl]-9-phenyl-9H-carbazole (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the above furan derivative (a compound having a furan skeleton) include compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DB TFLP-IV), 4,4′,4″-(benzene-1,3,5-triyl)tri(dibenzofuran) (abbreviation: DBF3P-II), and 4-{3-[3-(9-phenyl-9H-fluoren-9-yl)phenyl]phenyl}dibenzofuran (abbreviation: mmDBFFLBi-II).

Specific examples of the above aromatic amine include 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N′-phenyl-N′-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 2,7-bis[N-(4-diphenylaminophenyl)-N-phenylamino]-spiro-9,9′-bifluorene (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis {4-[bis(3-methylphenyl)amino]phenyl}-N,N′-diphenyl-(1,1′-biphenyl)-4,4′-diamine (abbreviation: DNTPD), and 1,3,5-tris[N-(4-diphenylaminophenyl)-N-phenylamino]benzene (abbreviation: DPA3B).

As the hole-transport material, a high molecular compound such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N-phenylamino}phenyl)methacrylamide] (abbreviation: PTPDMA), or poly[N,N′-bis(4-butylphenyl)-N,N′-bis(phenyl)benzidine] (abbreviation: Poly-TPD) can also be used.

Note that the hole-transport material is not limited to the above, and one of or a combination of various known materials may be used as the hole-transport material.

As the acceptor material used for the hole-injection layer 111, an oxide of a metal belonging to any of Group 4 to Group 8 of the periodic table can be used. As specific examples, molybdenum oxide, vanadium oxide, niobium oxide, tantalum oxide, chromium oxide, tungsten oxide, manganese oxide, and rhenium oxide can be given. Among these, molybdenum oxide is particularly preferable since it is stable in the air, has a low hygroscopic property, and is easy to handle. It is also possible to use any of the above-described organic acceptors.

Note that the hole-injection layer 111 can be formed by any of various known deposition methods, and can be formed by a vacuum evaporation method, for example.

### <Hole-Transport Layer>

The hole-transport layer 112 is a layer transporting holes, which are injected from the first electrode 101 through the hole-injection layer 111, to the light-emitting layer 113. Note that the hole-transport layer 112 is a layer containing a hole-transport material. Thus, for the hole-transport layer 112, a hole-transport material that can be used for the hole-injection layer 111 can be used.

Note that in the light-emitting device of one embodiment of the present invention, the same organic compound is preferably used for the hole-transport layer 112 and the light-emitting layer 113. This is because the use of the same organic compounds for the hole-transport layer 112 and the light-emitting layer 113 allows efficient hole transport from the hole-transport layer 112 to the light-emitting layer 113.

### <Light-Emitting Layer>

The light-emitting layer 113 is a layer containing a light-emitting substance. In the light-emitting device of one embodiment of the present invention, the light-emitting layer 113 includes a host material and a guest material; the third organic compound is used as the host material; and as the guest material, the first organic compound, which is a material (a fluorescent substance) having a function of converting singlet excitation energy into light emission, and the second organic compound, which is a material (a phosphorescent substance or a TADF material) having a function of converting triplet excitation energy into light emission, are used. The light-emitting substance that can be used for the light-emitting layer 113 is not particularly limited as long as the above condition is satisfied, and it is possible to use a substance that exhibits emission color of blue, purple, bluish purple, green, yellowish green, yellow, orange, red, or the like can be used as appropriate.

Note that a plurality of kinds of organic compounds may be used as host materials used for the light-emitting layer 113; alternatively, an exciplex formed by these compounds may be used. A substance that has an energy gap larger than that of the first organic compound and that of the second organic compound, which are used as the guest material, is preferably used as the third organic compound used as the host material. It is preferable that the lowest singlet excitation energy level (S1 level) of the third organic compound be higher than the S1 level of the first organic compound and that the lowest triplet excitation energy level (T1 level) of the third organic compound be higher than the T1 level of the first organic compound. Furthermore, the lowest triplet excitation energy level (T1 level) of the third organic compound is preferably higher than the T1 level of the second organic compound.

An organic compound such as the aforementioned hole-transport material that can be used in the hole-transport layer 112 or an electron-transport material described later that can be used in the electron-transport layer 114, or an exciplex formed by a plurality of kinds of organic compounds can be used as the one or more kinds of organic compounds used as the host material as long as requirements for the host material used in the light-emitting layer are satisfied. An exciplex (also referred to as Exciplex) whose excited state is formed by a plurality of kinds of organic compounds has an extremely small difference between the S1 level and the T1 level and functions as a TADF material that can convert triplet excitation energy into singlet excitation energy. As a combination of the plurality of kinds of organic compounds forming an exciplex, for example, it is preferable that one have a π-electron deficient heteroaromatic ring and the other have a π-electron rich heteroaromatic ring. A phosphorescent substance such as an iridium-, rhodium-, or platinum-based organometallic complex or a metal complex may be used as one of the combination forming an exciplex.

Note that the first organic compound and the second organic compound, which are used as the guest materials of the light-emitting layer 113, preferably exhibit different emission colors. Alternatively, complementary emission colors may be combined to obtain white light emission.

The material described in Embodiment 2 can be used as the first organic compound, which is the first guest material of the light-emitting layer 113 and has a function of converting singlet excitation energy into light emission, in the combination satisfying requirements for the guest materials used in the light-emitting layer. Examples of the second organic compound, which is the second guest material of the light-emitting layer 113 and has a function of converting triplet excitation energy into light emission, include a substance that emits phosphorescence (a phosphorescent substance) and a TADF material that exhibits thermally activated delayed fluorescence. Any of these materials can be used similarly in the combination satisfying the requirements for the guest materials used in the light-emitting layer. The lowest singlet excitation energy level (S1 level) of the first organic compound is higher than the T1 level of the second organic compound. That is, a peak wavelength in the emission spectrum of light emitted from the second organic compound is longer than that in the emission spectrum of light emitted from the first organic compound.

A phosphorescent substance refers to a compound that exhibits phosphorescence but does not exhibit fluorescence at a temperature higher than or equal to low temperatures (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (platinum complex), a rare earth metal complex, or the like. Specifically, a transition metal element is preferable and it is particularly preferable that a platinum group element (ruthenium (Ru), rhodium (Rh), palladium (Pd), osmium (Os), iridium (Jr), or platinum (Pt)), especially iridium, be contained, in which case the transition probability relating to direct transition between the singlet ground state and the triplet excited state can be increased.

As a phosphorescent substance that emits blue or green light and whose emission spectrum has a peak wavelength at greater than or equal to 450 nm and less than or equal to 570 nm, the following substances can be given.

For example, organometallic complexes having a 4H-triazole skeleton, such as tris{2-[5-(2-methylphenyl)-4-(2,6-dimethylphenyl)-4H-1,2,4-triazol-3-yl-κN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-prop yl-1H-1,2,4-triazolato)iridium(III) (abbreviation: [Ir(Prptz1-Me)₃]); organometallic complexes having an imidazole skeleton, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)); and the like can be given.

As a phosphorescent substance that exhibits green or yellow and whose emission spectrum has a peak wavelength at greater than or equal to 495 nm and less than or equal to 590 nm, the following substances can be given.

For example, organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis {4,6-dimethyl-2-[6-(2,6-dimethylphenyl)-4-pyrimidinyl-κN3]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), and bis[2-(2-pyridinyl-κN)phenyl-κC][2-(4-methyl-5-phenyl-2-pyridinyl-κN)phenyl-κC]; organometallic complexes such as bis(2,4-diphenyl-1,3-oxazolato-N,C^{2′})iridium(III) acetylacetonate (abbreviation: [Ir(dpo)₂(acac)]), bis {2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and rare earth metal complexes such as tris(acetylacetonato)(monophenanthroline)terbium(III) (abbreviation: [Tb(acac)₃(Phen)]) can be given.

As a phosphorescent substance that exhibits yellow or red and whose emission spectrum has a peak wavelength at greater than or equal to 570 nm and less than or equal to 750 nm, the following substances can be given.

For example, organometallic complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis[2-[5-(2,6-dimethylphenyl)-3-(3,5-dimethylphenyl)-2-pyrazinyl-κN]-4,6-dimethylphenyl-κC](2,2′,6,6′-tetramethyl-3,5-heptadionato-κ2O,O′)iridium(II) (abbreviation: [Ir(dmdppr-dmp)₂(dpm)]), (acetylacetonato)bis[2-methyl-3-phenylquinoxalinato-N,C^{2′}]iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-X²O,O′)iridium(III) (abbreviation: [Ir(dmpqn)₂(acac)]); platinum complexes such as 2,3,7,8,12,13,17,18-octaethyl-21H,23H-porphyrinplatinum(II) (abbreviation: [PtOEP]); and rare earth metal complexes such as tris(1,3-diphenyl-1,3-propanedionato)(monophenanthroline)europium(II) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]) can be given.

Any of materials shown below can be used as the TADF material. The TADF material refers to a material that has a small difference (preferably, less than or equal to 0.2 eV) between the S1 level and the T1 level, can up-convert triplet excited state into singlet excited state (reverse intersystem crossing) using a little thermal energy, and efficiently exhibits light emission (fluorescence) from the singlet excited state. The thermally activated delayed fluorescence is efficiently obtained under the condition where the difference in energy between the triplet excited energy level and the singlet excited energy level is greater than or equal to 0 eV and less than or equal to 0.2 eV, preferably greater than or equal to 0 eV and less than or equal to 0.1 eV. Delayed fluorescence by the TADF material refers to light emission having a spectrum similar to that of normal fluorescence and an extremely long lifetime. The lifetime is 1×10⁻⁶ seconds or longer, preferably 1×10⁻³ seconds or longer.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

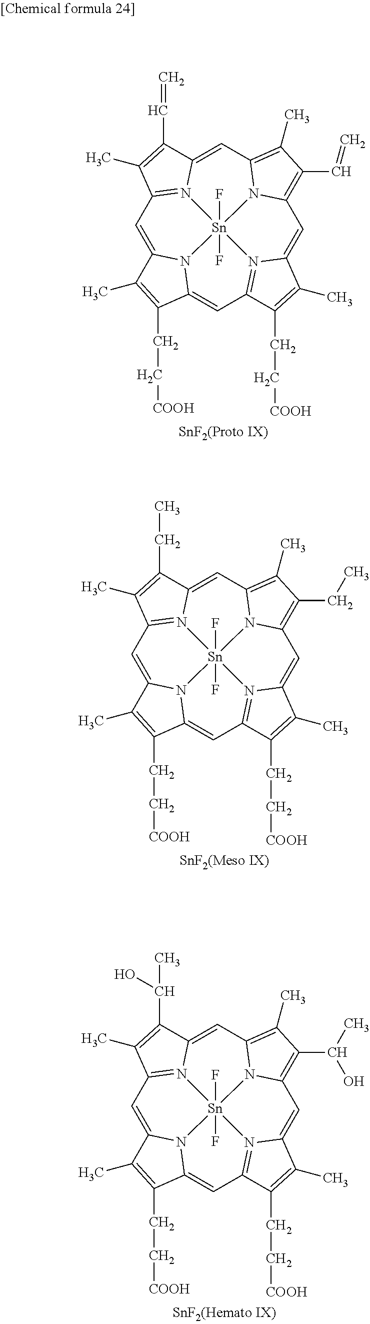

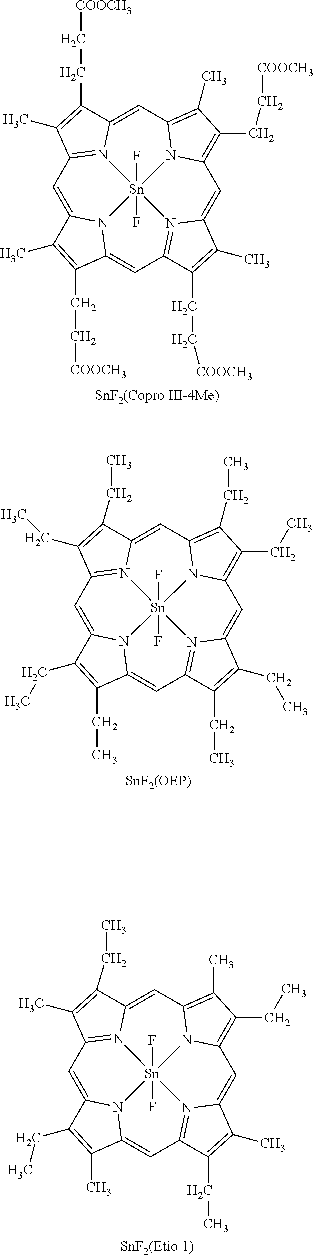

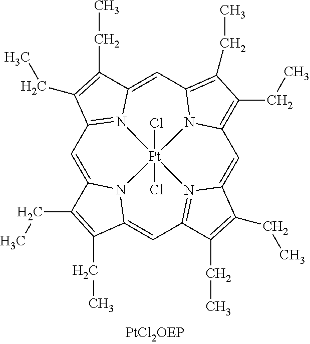

Alternatively, a heterocyclic compound having a π-electron rich heteroaromatic ring and a π-electron deficient heteroaromatic ring, such as 2-(biphenyl-4-yl)-4,6-bis(12-phenylindolo[2,3-α]carbazol-11-yl)-1,3,5-triazine (abbreviation: PIC-TRZ), 2-(4-[3-(N-phenyl-9H-carbazol-3-yl)-9H-carbazol-9-yl]phenyl)-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), and 9-[3-(4,6-diphenyl-1,3,5-triazin-2-yl)phenyl]-9′-phenyl-2,3′-bi-9H-carbazole (abbreviation: mPCCzPTzn-02), may be used.

Note that a substance in which a π-electron rich heteroaromatic ring is directly bonded to a π-electron deficient heteroaromatic ring is particularly preferable because both the donor property of the π-electron rich heteroaromatic ring and the acceptor property of the π-electron deficient heteroaromatic ring are improved and the energy difference between the singlet excited state and the triplet excited state becomes small.

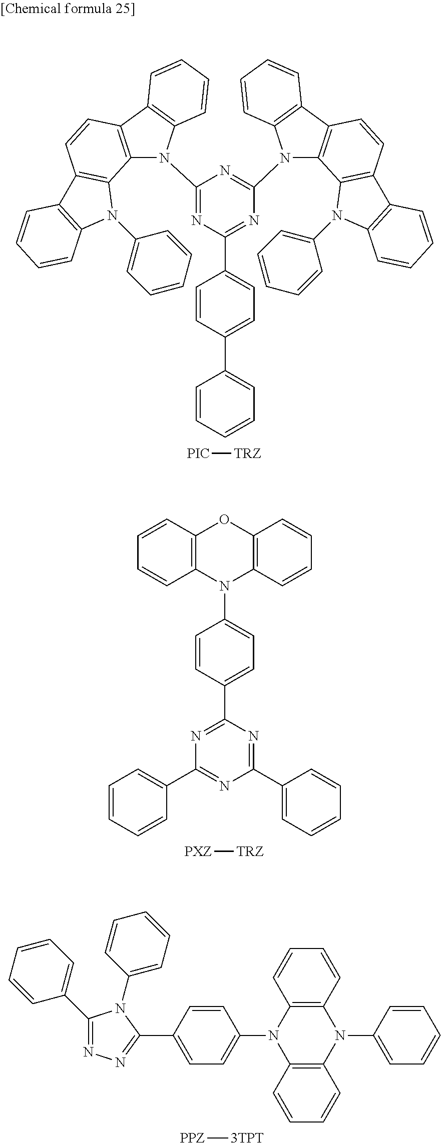

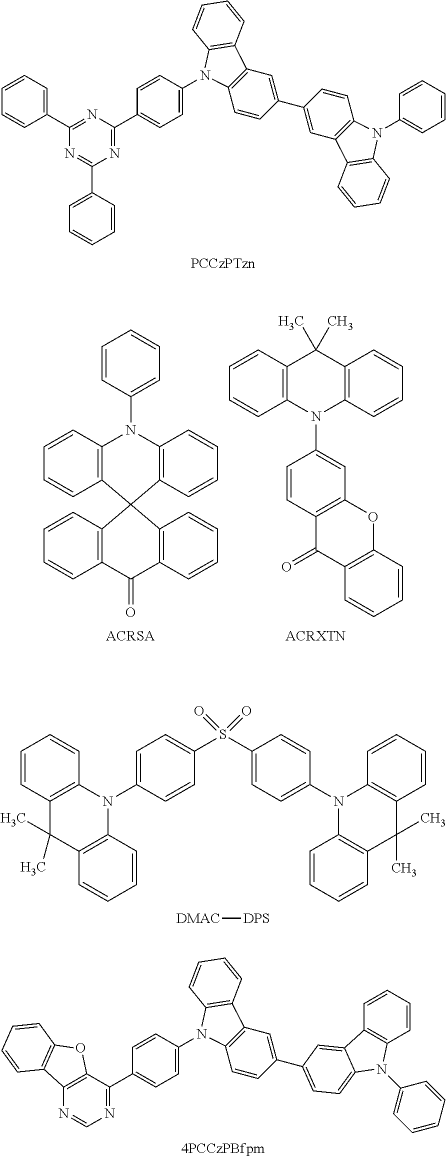

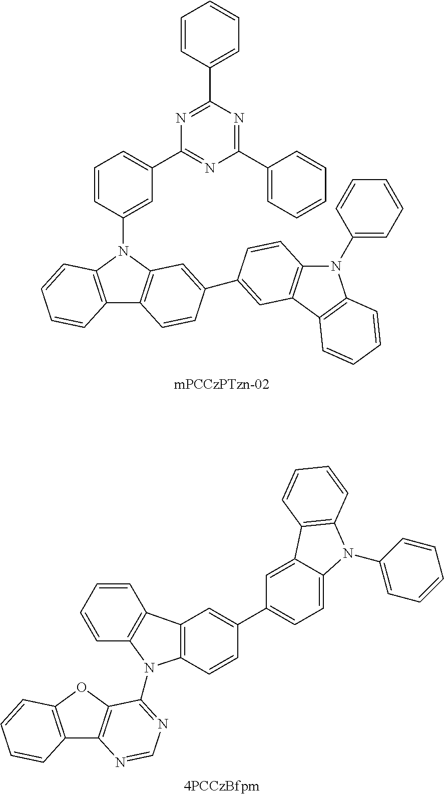 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-n2c3ccccc3c3cc(-c4ccc5c(c4)c4ccccc4n5-c4ncnc5c4oc4ccccc45)ccc32)cc1]

As the second organic compound, which is the material having a function of converting triplet excitation energy into light emission, a nanostructure of a transition metal compound having a perovskite structure is also given in addition to the above. In particular, a nanostructure of a metal-halide perovskite material is preferable. The nanostructure is preferably a nanoparticle or a nanorod.

Other than the above, the following substances emitting fluorescence (fluorescent substances) can be given as the light-emitting substance that can be used for the light-emitting layer 113 and convert singlet excitation energy into light emission. Examples include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of pyrene derivatives include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine] (abbreviation: 1,6BnfAPrn), N,N′-(pyrene-1,6-diyl)bis[(N-phenylbenzo[b]naphtho[1,2-d]furan)-8-amine] (abbreviation: 1,6BnfAPrn-02), and N,N′-(pyrene-1,6-diyl)bis[(6,N-diphenylbenzo[b]naphtho[1,2-d]furan)-8-amine] (abbreviation: 1,6BnfAPrn-03).

In addition, it is possible to use 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), or the like.

Examples of the third organic compound, which is the host material of the light-emitting layer 113, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the above include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)-phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N″,N″-octaphenyldibenzo[g,p]rysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-(4-(9-phenyl-9H-fluoren-9-yl)-biphenyl-4′-yl)-anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

In addition, examples of the third organic compound, which is the host material of the light-emitting layer 113, include an aromatic amine, a carbazole derivative, a dibenzothiophene derivative, a dibenzofuran derivative, a zinc- or aluminum-based metal complex, an oxadiazole derivative, a triazole derivative, a benzimidazole derivative, a quinoxaline derivative, a dibenzoquinoxaline derivative, a pyrimidine derivative, a pyrazine derivative, a triazine derivative, a pyridine derivative, a bipyridine derivative, and a phenanthroline derivative.

Specific examples include triazole derivatives such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), and 3-(4-tert-butylphenyl)-4-(4-ethylphenyl)-5-(4-biphenylyl)-1,2,4-triazole (abbreviation: p-EtTAZ); and quinoxaline derivatives or dibenzoquinoxaline derivatives, such as 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), 4,4′-bis(5-methylbenzoxazol-2-yl)stilbene (abbreviation: BzOs), bathophenanthroline (abbreviation: Bphen), bathocuproine (abbreviation: BCP), 2,9-bis(naphthalen-2-yl)-4,7-diphenyl-1,10-phenanthroline (abbreviation: NBphen), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[fh]quinoxaline (abbreviation: 2CzPDBq-III), 7-[3-(dibenzothiophen-4-yl)phenyl]dibenzo[f,h]quinoxaline (abbreviation: 7mDBTPDBq-II), and 6-[3-(dibenzothiophen-4-yl)phenyl]dibenzo[f,h]quinoxaline (abbreviation: 6mDBTPDBq-II).

Examples further include pyrimidine derivatives such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), triazine derivatives such as 2-{4-[3-(N-phenyl-9H-carbazol-3-yl)-9H-carbazol-9-yl]phenyl}-4,6-diphenyl-1,3,5-triazine (abbreviation: PCCzPTzn), triazine derivatives such as 9-[3-(4,6-diphenyl-1,3,5-triazin-2-yl)phenyl]-9′-phenyl-2,3′-bi-9H-carbazole (abbreviation: mPCCzPTzn-02), and pyridine derivatives such as 3,5-bis[3-(9H-carbazol-9-yl)phenyl]pyridine (abbreviation: 35DCzPPy), and 1,3,5-tri[3-(3-pyridyl)phenyl]benzene (abbreviation: TmPyPB).

Furthermore, a high molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy) can be used.

### <Electron-Transport Layer>

The electron-transport layer 114 is a layer transporting electrons, which are injected from the second electrode 102 through the electron-injection layer 115 to be described later, to the light-emitting layer 113. Note that the electron-transport layer 114 is a layer containing an electron-transport material. The electron-transport material used in the electron-transport layer 114 is preferably a substance having an electron mobility higher than or equal to 1×10⁻⁶ cm²/Vs. Note that other substances can be used as long as they have a property of transporting more electrons than holes. Electron-transport layers (114, 114a, and 114b) each function even with a single-layer structure, but can improve the device characteristics when having a stacked-layer structure of two or more layers as needed.

As the organic compound that can be used for the electron-transport layer 114, it is possible to use, in addition to the organic compounds having a structure in which an aromatic ring is fused to a furan ring of a furodiazine skeleton, a metal complex having a quinoline skeleton, a metal complex having a benzoquinoline skeleton, a metal complex having an oxazole skeleton, a metal complex having a thiazole skeleton, an oxadiazole derivative, a triazole derivative, an imidazole derivative, an oxazole derivative, a thiazole derivative, a phenanthroline derivative, a quinoline derivative having a quinoline ligand, a benzoquinoline derivative, a quinoxaline derivative, a dibenzoquinoxaline derivative, a pyridine derivative, a bipyridine derivative, a pyrimidine derivative, or a π-electron deficient heteroaromatic compound such as a nitrogen-containing heteroaromatic compound.

Specific examples of the electron-transport material include metal complexes having a quinoline skeleton or a benzoquinoline skeleton, such as 2-[3′-(dibenzothiophen-4-yl)biphenyl-3-yl]dibenzo[f,h]quinoxaline (abbreviation: 2mDBTBPDBq-II), 5-[3-(4,6-diphenyl-1,3,5-triazin-2yl)phenyl1]-7,7-dimethyl-5H,7H-indeno[2,1-b]carbazole (abbreviation: mINc(II)PTzn), 4-[3-(dibenzothiophen-4-yl)phenyl]-8-(naphthalen-2-yl)-[1]benzofuro[3,2-d]pyrimidine (abbreviation: 8βN-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 9-[(3′-dibenzothiophen-4-yl)biphenyl-3-yl]naphtho[1′,2′: 4,5]furo[2,3-b]pyrazine (abbreviation: 9mDBtBPNfpr), 8-[3′-(dibenzothiophen-4-yl) (1,1′-biphenyl-3-yl)]naphtho[1′,2′: 4,5]furo[3,2-d]pyrimidine (abbreviation: 8mDBtBPNfpm), 8-[(2,2′-binaphthalen)-6-yl]-4-[3-(dibenzothiophen-4-yl]phenyl-[1]benzofuro[3,2-d]pyrimidine (abbreviation: 8(βN2)-4mDBtPBfpm), tris(8-quinolinolato)aluminum(III) (abbreviation: Alq₃), tris(4-methyl-8-quinolinolato)aluminum(III) (abbreviation: Almq₃), bis(10-hydroxybenzo[h]quinolinato)beryllium (abbreviation: BeBq₂), bis(2-methyl-8-quinolinolato) (4-phenylphenolato)aluminum(III) (abbreviation: BAlq), and bis(8-quinolinolato)zinc(II) (abbreviation: Znq), and metal complexes having an oxazole skeleton or a thiazole skeleton, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Other than the metal complexes, it is possible to use oxadiazole derivatives such as PBD, OXD-7, and CO11, triazole derivatives such as TAZ and p-EtTAZ, imidazole derivatives (including benzimidazole derivatives) such as TPBI and mDBTBIm-II, an oxazole derivative such as BzOs, phenanthroline derivatives such as Bphen, BCP, and NBphen, quinoxaline derivatives and dibenzoquinoxaline derivatives, such as 2mDBTPDBq-II, 2mDBTBPDBq-II, 2mCzBPDBq, 2CzPDBq-III, 7mDBTPDBq-II, and 6mDBTPDBq-II, pyridine derivatives such as 35DCzPPy and TmPyPB, pyrimidine derivatives such as 4,6mPnP2Pm, 4,6mDBTP2Pm-II, and 4,6mCzP2Pm, and triazine derivatives such as PCCzPTzn and mPCCzPTzn-02.

It is also possible to use high molecular compounds such as PPy, PF-Py, and PF-BPy.

### <Electron-Injection Layer>

The electron-injection layer 115 is a layer for increasing the efficiency of electron injection from the cathode 102, and thus is preferably formed using a material whose LUMO level value has a small difference (0.5 eV or less) from the work function value of a material of the second electrode (cathode) 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), 8-(quinolinolato)lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), lithium oxide (LiOₓ), or cesium carbonate. A rare earth metal compound like erbium fluoride (ErF₃) can also be used.

When a charge-generation layer 104 is provided between two EL layers (103a and 103b) as in the light-emitting device shown in FIG. 6B, a structure in which a plurality of EL layers are stacked between the pair of electrodes (also referred to as a tandem structure) can be employed. Note that in this embodiment, functions and materials of the hole-injection layer (111), the hole-transport layer (112), the light-emitting layer (113), the electron-transport layer (114), and the electron-injection layer (115) that are illustrated in FIG. 6A are the same as those of hole-injection layers (111a and 111b), hole-transport layers (112a and 112b), light-emitting layers (113a and 113b), the electron-transport layers (114a and 114b), and electron-injection layers (115a and 115b) that are illustrated in FIG. 6B.

### <Charge-Generation Layer>

In the light-emitting device of FIG. 6B, the charge-generation layer 104 has a function of injecting electrons into the EL layer 103a and injecting holes into the EL layer 103b when voltage is applied between the first electrode (anode) 101 and the second electrode (cathode) 102. Note that the charge-generation layer 104 may have either a structure in which an electron acceptor (acceptor) is added to a hole-transport material or a structure in which an electron donor (donor) is added to an electron-transport material. Alternatively, both of these structures may be stacked. Note that forming the charge-generation layer 104 with the use of any of the above materials can inhibit an increase in drive voltage in the case where the EL layers are stacked.

In the case where the charge-generation layer 104 has a structure in which an electron acceptor is added to a hole-transport material, any of the materials described in this embodiment can be used as the hole-transport material. As the electron acceptor, 7,7,8,8-tetracyano-2,3,5,6-tetrafluoroquinodimethane (abbreviation: F₄-TCNQ), chloranil, and the like can be given. Other examples include oxides of metals belonging to Group 4 to Group 8 of the periodic table. Specific examples are vanadium oxide, niobium oxide, tantalum oxide, chromium oxide, molybdenum oxide, tungsten oxide, manganese oxide, and rhenium oxide.

In the case where the charge-generation layer 104 has a structure in which an electron donor is added to an electron-transport material, any of the materials described in this embodiment can be used as the electron-transport material. As the electron donor, it is possible to use an alkali metal, an alkaline earth metal, a rare earth metal, metals belonging to Group 2 and Group 13 of the periodic table, or an oxide or carbonate thereof. Specifically, lithium (Li), cesium (Cs), magnesium (Mg), calcium (Ca), ytterbium (Yb), indium (In), lithium oxide, cesium carbonate, or the like is preferably used. An organic compound such as tetrathianaphthacene may be used as the electron donor.

Although FIG. 6B shows the structure in which two EL layers 103 are stacked, a structure may be employed in which three or more EL layers are stacked with a charge-generation layer provided between different EL layers.

### <Substrate>

The light-emitting device described in this embodiment can be formed over any of a variety of substrates. Note that the type of the substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, a laminate film, paper including a fibrous material, and a base material film.

Note that examples of the glass substrate include barium borosilicate glass, aluminoborosilicate glass, and soda lime glass. Examples of the flexible substrate, the laminate film, and the base material film include plastics typified by polyethylene terephthalate (PET), polyethylene naphthalate (PEN), and polyether sulfone (PES); a synthetic resin such as an acrylic resin; polypropylene; polyester; polyvinyl fluoride; polyvinyl chloride; polyamide; polyimide; an aramid resin; an epoxy resin; an inorganic vapor deposition film; and paper.

For fabrication of the light-emitting device in this embodiment, a vacuum process such as an evaporation method or a solution process such as a spin coating method or an ink-jet method can be used. In the case of using an evaporation method, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the functional layers (the hole-injection layers (111, 111a, and 111b), the hole-transport layers (112, 112a, and 112b), the light-emitting layers (113, 113a, and 113b), the electron-transport layers (114, 114a, and 114b), and the electron-injection layers (115, 115a, and 115b)) included in the EL layers and the charge-generation layer 104 of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, a screen printing (stencil) method, an offset printing (planography) method, a flexography (relief printing) method, a gravure printing method, a micro-contact printing method, or a nanoimprinting method), or the like.

Note that materials that can be used for the functional layers (the hole-injection layers (111, 111a, and 111b), the hole-transport layers (112, 112a, and 112b), the light-emitting layers (113, 113a, and 113b), the electron-transport layers (114, 114a, and 114b), and the electron-injection layers (115, 115a, and 115b)) included in the EL layers (103, 103a, and 103b) and the charge-generation layer 104 of the light-emitting device described in this embodiment are not limited to the above materials, and other materials can also be used in combination as long as the functions of the layers are fulfilled. For example, a high molecular compound (e.g., an oligomer, a dendrimer, and a polymer), a middle molecular compound (a compound between a low molecular compound and a high molecular compound with a molecular weight of 400 to 4000), or an inorganic compound (e.g., a quantum dot material) can be used. As the quantum dot material, a colloidal quantum dot material, an alloyed quantum dot material, a core-shell quantum dot material, a core quantum dot material, or the like can be used.

The structure described in this embodiment can be used in an appropriate combination with any of the structures described in the other embodiments.

### Embodiment 4

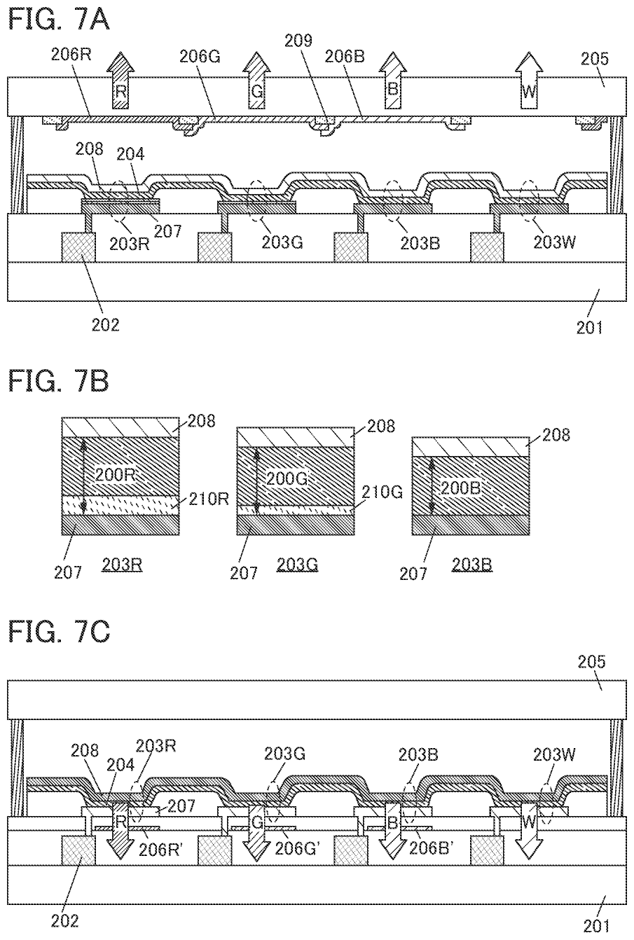

In this embodiment, a light-emitting apparatus of one embodiment of the present invention will be described. A light-emitting apparatus shown in FIG. 7A is an active-matrix light-emitting apparatus in which transistors (FETs) 202 over a first substrate 201 are electrically connected to light-emitting devices (203R, 203G, 203B, and 203W); the light-emitting devices (203R, 203G, 203B, and 203W) include a common EL layer 204 and each have a microcavity structure where the optical path length between electrodes of each light-emitting device is adjusted so that light of the desired color is emitted by the light-emitting device. The light-emitting apparatus is a top-emission light-emitting apparatus in which light obtained from the EL layer 204 is emitted through color filters (206R, 206G, and 206B) formed on a second substrate 205.

In the light-emitting apparatus shown in FIG. 7A, a first electrode 207 is formed so as to function as a reflective electrode. A second electrode 208 is formed to function as a transflective electrode having both properties of transmitting and reflecting light (visible light or near-infrared light). Note that the description in the other embodiments can be referred to for electrode materials forming the first electrode 207 and the second electrode 208, and appropriate materials can be used.

In the case where the light-emitting device 203R is a red-light-emitting device, the light-emitting device 203G is a green-light-emitting device, the light-emitting device 203B is a blue-light-emitting device, and the light-emitting device 203W is a white-light-emitting device in FIG. 7A, for example, the distance between the first electrode 207 and the second electrode 208 in the light-emitting device 203R is adjusted to have an optical path length 200R, the distance between the first electrode 207 and the second electrode 208 in the light-emitting device 203G is adjusted to have an optical path length 200G, and the distance between the first electrode 207 and the second electrode 208 in the light-emitting device 203B is adjusted to have an optical path length 200B, as shown in FIG. 7B. Note that optical adjustment can be performed in such a manner that a conductive layer 210R is stacked over the first electrode 207 in the light-emitting device 203R and a conductive layer 210G is stacked in the light-emitting device 203G as shown in FIG. 7B.

The color filters (206R, 206G, and 206B) are formed on the second substrate 205. Note that the color filter is a filter that transmits visible light in a specific wavelength range and blocks visible light in a specific wavelength range. Thus, as shown in FIG. 7A, the color filter 206R that transmits only light in the red wavelength range is provided in a position overlapping with the light-emitting device 203R, whereby red light emission can be obtained from the light-emitting device 203R. The color filter 206G that transmits only light in the green wavelength range is provided in a position overlapping with the light-emitting device 203G, whereby green light emission can be obtained from the light-emitting device 203G. The color filter 206B that transmits only light in the blue wavelength range is provided in a position overlapping with the light-emitting device 203B, whereby blue light emission can be obtained from the light-emitting device 203B. The light-emitting device 203W can emit white light without a color filter. Note that a black layer (black matrix) 209 may be provided at an end portion of one type of color filter. The color filters (206R, 206G, and 206B) and the black layer 209 may be covered with an overcoat layer using a transparent material.

Although the light-emitting apparatus in FIG. 7A has a structure in which light is extracted from the second substrate 205 side (a top-emission structure), the light-emitting apparatus may have a structure in which light is extracted from the first substrate 201 side where the FETs 202 are formed (a bottom-emission structure) as shown in FIG. 7C. For a bottom-emission light-emitting apparatus, the first electrode 207 is formed so as to function as a transflective electrode and the second electrode 208 is formed so as to function as a reflective electrode. As the first substrate 201, a substrate having at least a light-transmitting property is used. As shown in FIG. 7C, color filters (206R′, 206G′, and 206B′) are provided closer to the first substrate 201 than the light-emitting devices (203R, 203G, and 203B) are.

FIG. 7A shows the case where the light-emitting devices are the red-light-emitting device, the green-light-emitting device, the blue-light-emitting device, and the white-light-emitting device; however, the light-emitting devices of embodiments of the present invention are not limited to the above structures, and a yellow-light-emitting device or an orange-light-emitting device may be included. Note that the description in the other embodiments can be referred to for materials that are used for the EL layers (a light-emitting layer, a hole-injection layer, a hole-transport layer, an electron-transport layer, an electron-injection layer, a charge-generation layer, and the like) to fabricate each of the light-emitting devices, and appropriate materials can be used. In that case, a color filter needs to be appropriately selected according to the emission color of the light-emitting device.

With the above structure, a light-emitting apparatus including light-emitting devices that exhibit a plurality of emission colors can be obtained.

Note that the structure described in this embodiment can be used in an appropriate combination with any of the structures described in the other embodiments.

### Embodiment 5

In this embodiment, a light-emitting apparatus of one embodiment of the present invention will be described.

The use of the device structure of the light-emitting device of one embodiment of the present invention allows fabrication of an active-matrix light-emitting apparatus and a passive-matrix light-emitting apparatus. Note that an active-matrix light-emitting apparatus has a structure including a combination of a light-emitting device and a transistor (an FET). Thus, each of a passive-matrix light-emitting apparatus and an active-matrix light-emitting apparatus is included in one embodiment of the present invention. Note that any of the light-emitting devices described in the other embodiments can be used in the light-emitting apparatus described in this embodiment.

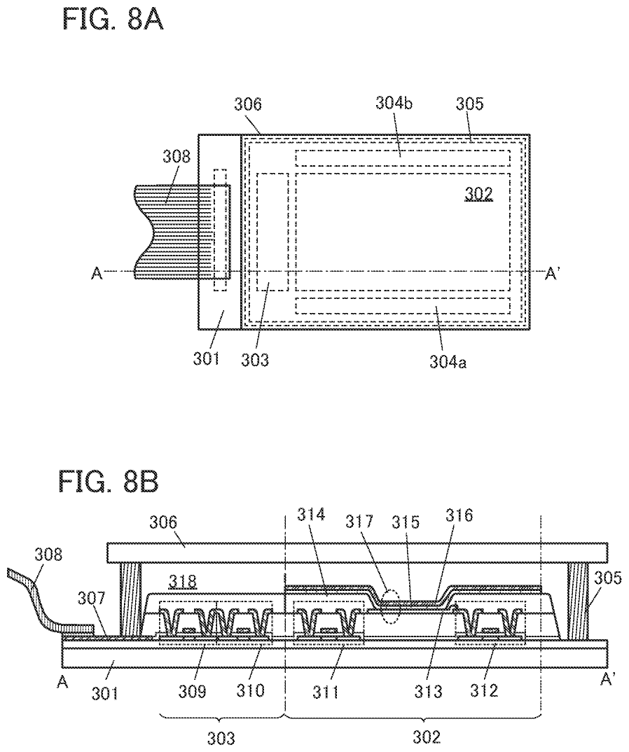

In this embodiment, an active-matrix light-emitting apparatus will be described with reference to FIG. 8.

FIG. 8A is a top view showing a light-emitting apparatus, and FIG. 8B is a cross-sectional view taken along a chain line A-A′ in FIG. 8A. The active-matrix light-emitting apparatus includes a pixel portion 302, a driver circuit portion (source line driver circuit) 303, and driver circuit portions (gate line driver circuits) (304a and 304b) that are provided over a first substrate 301. The pixel portion 302 and the driver circuit portions (303, 304a, and 304b) are sealed between the first substrate 301 and a second substrate 306 with a sealant 305.

A lead wiring 307 is provided over the first substrate 301. The lead wiring 307 is electrically connected to an FPC 308 that is an external input terminal. The FPC 308 transmits a signal (e.g., a video signal, a clock signal, a start signal, and a reset signal) and a potential from the outside to the driver circuit portions (303, 304a, and 304b). The FPC 308 may be provided with a printed wiring board (PWB). Note that the light-emitting apparatus provided with an FPC or a PWB is included in the category of a light-emitting apparatus.

Next, FIG. 8B shows the cross-sectional structure.

The pixel portion 302 is made up of a plurality of pixels each including an FET (switching FET) 311, an FET (current control FET) 312, and a first electrode 313 electrically connected to the FET 312. Note that the number of FETs included in each pixel is not particularly limited and can be set appropriately as needed.

As FETs 309, 310, 311, and 312, for example, a staggered transistor or an inverted staggered transistor can be used without particular limitation. A top-gate transistor, a bottom-gate transistor, or the like may be used.

Note that there is no particular limitation on the crystallinity of a semiconductor that can be used for the FETs 309, 310, 311, and 312, and an amorphous semiconductor or a semiconductor having crystallinity (a microcrystalline semiconductor, a polycrystalline semiconductor, a single crystal semiconductor, or a semiconductor partly including crystal regions) may be used. The use of a semiconductor having crystallinity is preferable, in which case deterioration of the transistor characteristics can be inhibited.

For the semiconductor, a Group 14 element, a compound semiconductor, an oxide semiconductor, an organic semiconductor, or the like can be used, for example. Typically, a semiconductor containing silicon, a semiconductor containing gallium arsenide, an oxide semiconductor containing indium, or the like can be used.

The driver circuit portion 303 includes the FET 309 and the FET 310. The driver circuit portion 303 may be formed with a circuit including transistors having the same conductivity type (either only n-channel transistors or only p-channel transistors) or a CMOS circuit including an n-channel transistor and a p-channel transistor. Furthermore, a driver circuit may be provided outside.

An end portion of the first electrode 313 is covered with an insulator 314. For the insulator 314, an organic compound such as a negative photosensitive resin or a positive photosensitive resin (an acrylic resin), or an inorganic compound such as silicon oxide, silicon oxynitride, or silicon nitride can be used. An upper end portion or a lower end portion of the insulator 314 preferably has a curved surface with curvature. In that case, favorable coverage with a film formed over the insulator 314 can be obtained.

An EL layer 315 and a second electrode 316 are stacked over the first electrode 313. The EL layer 315 includes a light-emitting layer, a hole-injection layer, a hole-transport layer, an electron-transport layer, an electron-injection layer, a charge-generation layer, and the like.

The structure and materials described in the other embodiments can be used for the structure of a light-emitting device 317 described in this embodiment. Although not shown here, the second electrode 316 is electrically connected to the FPC 308 that is an external input terminal.

Although the cross-sectional view in FIG. 8B shows only one light-emitting device 317, a plurality of light-emitting devices are arranged in a matrix in the pixel portion 302. Light-emitting devices from which light of three kinds of colors (R, G, and B) is obtained are selectively formed in the pixel portion 302, whereby a light-emitting apparatus capable of full-color display can be formed. In addition to the light-emitting devices from which light of three kinds of colors (R, G, and B) is obtained, for example, light-emitting devices from which light of white (W), yellow (Y), magenta (M), cyan (C), and the like is obtained may be formed. For example, when the light-emitting devices from which light of some of the above colors is obtained are added to the light-emitting devices from which light of three kinds of colors (R, G, and B) is obtained, effects such as an improvement in color purity and a reduction in power consumption can be obtained. Alternatively, a light-emitting apparatus that is capable of full-color display may be fabricated by a combination with color filters. As the kinds of color filters, red (R), green (G), blue (B), cyan (C), magenta (M), and yellow (Y) color filters and the like can be used.

When the second substrate 306 and the first substrate 301 are bonded to each other with the sealant 305, the FETs (309, 310, 311, and 312) and the light-emitting device 317 over the first substrate 301 are provided in a space 318 surrounded by the first substrate 301, the second substrate 306, and the sealant 305. Note that the space 318 may be filled with an inert gas (e.g., nitrogen or argon) or an organic substance (including the sealant 305).

An epoxy resin or glass frit can be used for the sealant 305. A material that transmits moisture and oxygen as little as possible is preferably used for the sealant 305. As the second substrate 306, a substrate that can be used as the first substrate 301 can be similarly used. Thus, any of the various substrates described in the other embodiments can be appropriately used. As the substrate, a glass substrate, a quartz substrate, or a plastic substrate made of FRP (Fiber-Reinforced Plastics), PVF (polyvinyl fluoride), polyester, an acrylic resin, or the like can be used. In the case where glass frit is used for the sealant, the first substrate 301 and the second substrate 306 are preferably glass substrates in terms of adhesion.

In the above manner, the active-matrix light-emitting apparatus can be obtained.

In the case where the active-matrix light-emitting apparatus is formed over a flexible substrate, the FETs and the light-emitting device may be directly formed over the flexible substrate; alternatively, the FETs and the light-emitting device may be formed over a substrate provided with a separation layer and then separated at the separation layer by application of heat, force, laser irradiation, or the like to be transferred to a flexible substrate. For the separation layer, a stack of inorganic films such as a tungsten film and a silicon oxide film, or an organic resin film of polyimide or the like can be used, for example. Examples of the flexible substrate include, in addition to a substrate where a transistor can be formed, a paper substrate, a cellophane substrate, an aramid film substrate, a polyimide film substrate, a cloth substrate (including a natural fiber (silk, cotton, and hemp), a synthetic fiber (nylon, polyurethane, and polyester), a regenerated fiber (acetate, cupro, rayon, and regenerated polyester), and the like), a leather substrate, and a rubber substrate. With the use of any of these substrates, high durability, high heat resistance, a reduction in weight, and a reduction in thickness can be achieved.

The light-emitting device included in the active-matrix light-emitting apparatus may be driven to emit light in a pulsed manner (using a frequency of kHz or MHz, for example) so that the light is used for display. The light-emitting device formed using any of the above organic compounds has excellent frequency characteristics; thus, the time for driving the light-emitting device can be shortened, and the power consumption can be reduced. Furthermore, a reduction in driving time leads to inhibition of heat generation, so that the degree of deterioration of the light-emitting device can be reduced.

Note that the structure described in this embodiment can be used in an appropriate combination with any of the structures described in the other embodiments.

### Embodiment 6

In this embodiment, examples of a variety of electronic devices and an automobile completed using the light-emitting device of one embodiment of the present invention or a light-emitting apparatus including the light-emitting device of one embodiment of the present invention will be described. Note that the light-emitting apparatus can be used mainly in a display portion of the electronic device described in this embodiment.

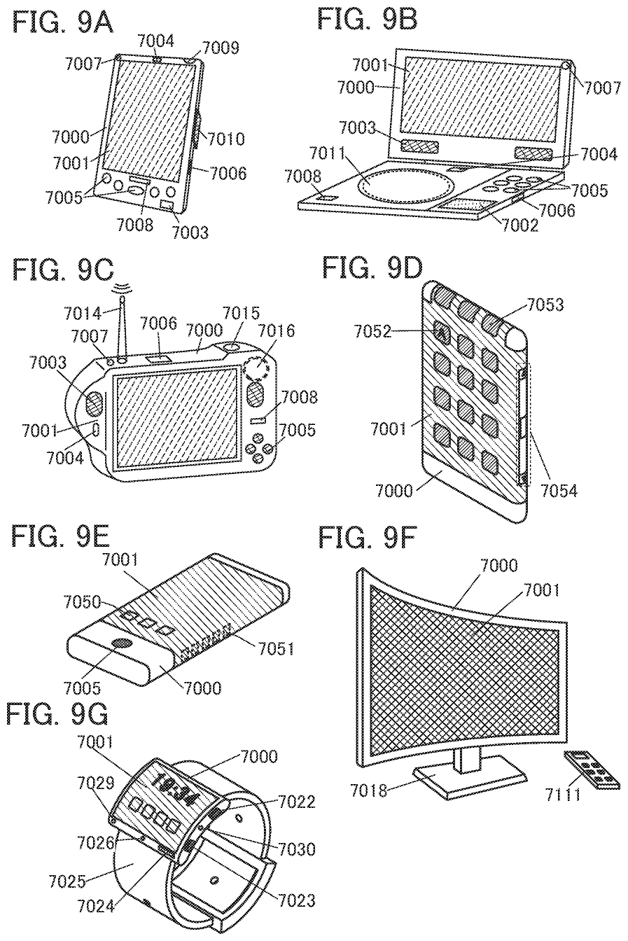

Electronic devices shown in FIG. 9A to FIG. 9E can include a housing 7000, a display portion 7001, a speaker 7003, an LED lamp 7004, an operation key 7005 (including a power switch or an operation switch), a connection terminal 7006, a sensor 7007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 7008, and the like.

FIG. 9A is a mobile computer that can include a switch 7009, an infrared port 7010, and the like in addition to the above components.

FIG. 9B is a portable image reproducing device (e.g., a DVD player) that is provided with a recording medium and can include a second display portion 7002, a recording medium reading portion 7011, and the like in addition to the above components.

FIG. 9C is a digital camera that has a television reception function and can include an antenna 7014, a shutter button 7015, an image receiving portion 7016, and the like in addition to the above components.

FIG. 9D is a portable information terminal. The portable information terminal has a function of displaying information on three or more surfaces of the display portion 7001. Here, an example in which information 7052, information 7053, and information 7054 are displayed on different surfaces is shown. For example, the user can check the information 7053 displayed in a position that can be observed from above the portable information terminal, with the portable information terminal put in a breast pocket of his/her clothes. The user can see the display without taking out the portable information terminal from the pocket and decide whether to answer the call, for example.

FIG. 9E is a portable information terminal (e.g., a smartphone) that can include the display portion 7001, the operation key 7005, and the like in the housing 7000. Note that the speaker, the connection terminal, the sensor, or the like may be provided in the portable information terminal. The portable information terminal can display text and image information on its plurality of surfaces. Here, an example in which three icons 7050 are displayed is shown. Information 7051 indicated by dashed rectangles can be displayed on another surface of the display portion 7001. Examples of the information 7051 include notification of reception of an e-mail, SNS, or an incoming call, the title and sender of an e-mail, SNS, or the like, the date, the time, remaining battery, and the reception strength of an antenna. Alternatively, the icon 7050 or the like may be displayed in the position where the information 7051 is displayed.

FIG. 9F is a large-size television set (also referred to as TV or television receiver) that can include the housing 7000, the display portion 7001, and the like. Here, a structure in which the housing 7000 is supported by a stand 7018 is shown. The television set can be operated with a separate remote controller 7111 or the like. Note that the display portion 7001 may include a touch sensor, in which case the television set may be operated by touch on the display portion 7001 with a finger or the like. The remote controller 7111 may include a display portion for displaying information output from the remote controller 7111. With operation keys or a touch panel provided in the remote controller 7111, channels and volume can be controlled and images displayed on the display portion 7001 can be controlled.

The electronic devices shown in FIG. 9A to FIG. 9F can have a variety of functions. For example, they can have a function of displaying a variety of information (e.g., a still image, a moving image, and a text image) on the display portion, a touch panel function, a function of displaying a calendar, date, time, or the like, a function of controlling processing with a variety of software (programs), a wireless communication function, a function of being connected to a variety of computer networks with a wireless communication function, a function of transmitting and receiving a variety of data with a wireless communication function, and a function of reading out a program or data stored in a recording medium and displaying it on the display portion. Furthermore, the electronic device including a plurality of display portions can have a function of displaying image information mainly on one display portion while displaying text information mainly on another display portion, a function of displaying a three-dimensional image by displaying images on a plurality of display portions with a parallax taken into account, or the like. The electronic device including an image receiving portion can have a function of taking a still image, a function of taking a moving image, a function of automatically or manually correcting a taken image, a function of storing a taken image in a recording medium (an external recording medium or a recording medium incorporated in the camera), a function of displaying a taken image on the display portion, and the like. Note that functions that the electronic devices shown in FIG. 9A to FIG. 9F can have are not limited to those, and the electronic devices can have a variety of functions.

FIG. 9G is a watch-type portable information terminal that can be used as a watch-type electronic device, for example. The watch-type portable information terminal includes the housing 7000, the display portion 7001, operation buttons 7022 and 7023, a connection terminal 7024, a band 7025, a microphone 7026, a sensor 7029, a speaker 7030, and the like. The display surface of the display portion 7001 is bent, and display can be performed along the bent display surface. The portable information terminal enables hands-free calling by mutually communicating with, for example, a headset capable of wireless communication. With the connection terminal 7024, the portable information terminal can perform mutual data transmission with another information terminal and be charged. Wireless power feeding can also be employed for the charging operation.

The display portion 7001 mounted in the housing 7000 also serving as a bezel includes a non-rectangular display region. The display portion 7001 can display an icon indicating time, another icon, and the like. The display portion 7001 may be a touch panel (an input/output device) including a touch sensor (an input device).

Note that the watch-type electronic device shown in FIG. 9G can have a variety of functions. For example, it can have a function of displaying a variety of information (e.g., a still image, a moving image, and a text image) on the display portion, a touch panel function, a function of displaying a calendar, date, time, or the like, a function of controlling processing with a variety of software (programs), a wireless communication function, a function of being connected to a variety of computer networks with a wireless communication function, a function of transmitting and receiving a variety of data with a wireless communication function, and a function of reading out a program or data stored in a recording medium and displaying it on the display portion.

Moreover, a speaker, a sensor (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone, and the like can be included inside the housing 7000.

Note that the light-emitting apparatus of one embodiment of the present invention can be used in the display portions of the electronic devices described in this embodiment, enabling the electronic devices to have a long lifetime.

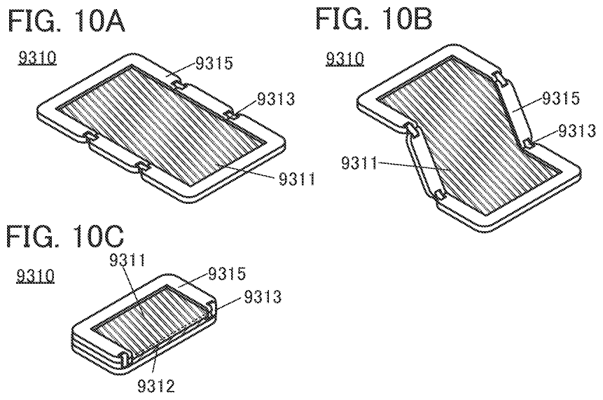

Another electronic device including the light-emitting apparatus is a foldable portable information terminal shown in FIG. 10A to FIG. 10C. FIG. 10A shows a portable information terminal 9310 that is opened. FIG. 10B shows the portable information terminal 9310 in a state in the middle of change from one of an opened state and a folded state to the other. FIG. 10C shows the portable information terminal 9310 that is folded. The portable information terminal 9310 is excellent in portability when folded, and is excellent in display browsability when opened because of a seamless large display region.

A display portion 9311 is supported by three housings 9315 joined together by hinges 9313. Note that the display portion 9311 may be a touch panel (an input/output device) including a touch sensor (an input device). By bending the display portion 9311 at a portion between two housings 9315 with the use of the hinges 9313, the portable information terminal 9310 can be reversibly changed in shape from an opened state to a folded state. The light-emitting apparatus of one embodiment of the present invention can be used in the display portion 9311. An electronic device having a long lifetime can be achieved. A display region 9312 in the display portion 9311 is a display region that is positioned at a side surface of the portable information terminal 9310 that is folded. On the display region 9312, information icons, file shortcuts of frequently used applications and programs, and the like can be displayed; hence, confirmation of information and start of an application can be smoothly performed.

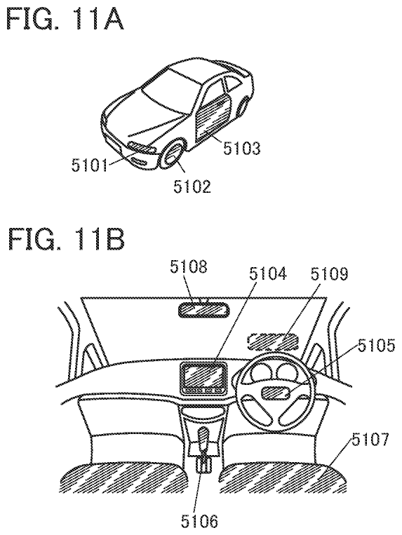

FIG. 11A and FIG. 11B show an automobile including the light-emitting apparatus. In other words, the light-emitting apparatus can be integrated into an automobile. Specifically, the light-emitting apparatus can be applied to lights 5101 (including lights of the rear part of the car), a tire wheel 5102, a part or the whole of a door 5103, or the like on the outer side of the automobile shown in FIG. 11A. The light-emitting apparatus can also be applied to a display portion 5104, a steering wheel 5105, a shifter 5106, a seat 5107, an inner rearview mirror 5108, a windshield 5109, or the like on the inner side of the automobile shown in FIG. 11B. The light-emitting apparatus may be applied to part of any of the other glass windows.

In the above manner, the electronic devices and the automobile each including the light-emitting apparatus of one embodiment of the present invention can be obtained. In that case, a long-lifetime electronic device can be achieved. In addition, the light-emitting apparatus can be used for electronic devices and automobiles in a variety of fields without being limited to those described in this embodiment.

Note that the structure described in this embodiment can be used in an appropriate combination with any of the structures described in the other embodiments.

### Embodiment 7

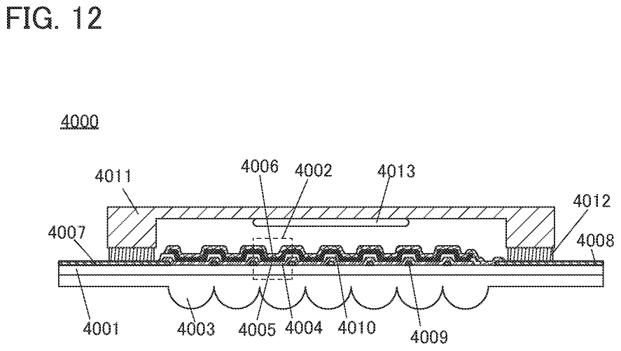

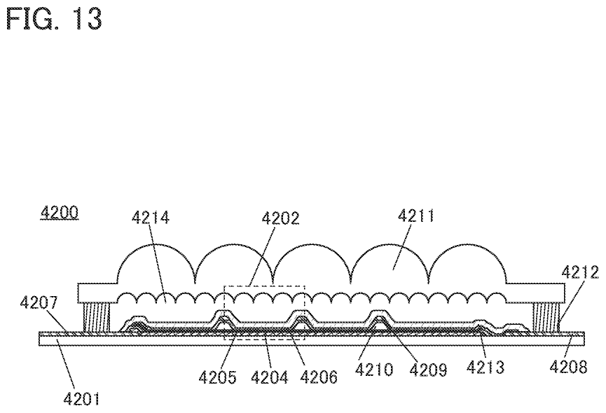

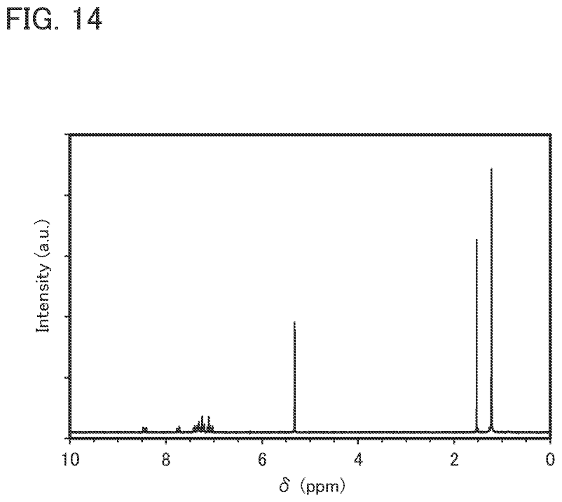

In this embodiment, a structure of a lighting device fabricated using the light-emitting apparatus of one embodiment of the present invention or the light-emitting device which is part of the light-emitting apparatus will be described with reference to FIG. 12 and FIG. 13.

FIG. 12 and FIG. 13 show examples of cross-sectional views of lighting devices. FIG. 12 illustrates a bottom-emission lighting device in which light is extracted from the substrate side, and FIG. 13 illustrates a top-emission lighting device in which light is extracted from the sealing substrate side.

A lighting device 4000 shown in FIG. 12 includes a light-emitting device 4002 over a substrate 4001. In addition, the lighting device 4000 includes a substrate 4003 with unevenness on the outer side of the substrate 4001. The light-emitting device 4002 includes a first electrode 4004, an EL layer 4005, and a second electrode 4006.

The first electrode 4004 is electrically connected to an electrode 4007, and the second electrode 4006 is electrically connected to an electrode 4008. An auxiliary wiring 4009 electrically connected to the first electrode 4004 may be provided. An insulating layer 4010 is formed over the auxiliary wiring 4009.

The substrate 4001 and a sealing substrate 4011 are bonded to each other with a sealant 4012. A desiccant 4013 is preferably provided between the sealing substrate 4011 and the light-emitting device 4002. Since the substrate 4003 has the unevenness shown in FIG. 12, the extraction efficiency of light generated in the light-emitting device 4002 can be increased.

A lighting device 4200 in FIG. 13 includes a light-emitting device 4202 over a substrate 4201. The light-emitting device 4202 includes a first electrode 4204, an EL layer 4205, and a second electrode 4206.

The first electrode 4204 is electrically connected to an electrode 4207, and the second electrode 4206 is electrically connected to an electrode 4208. An auxiliary wiring 4209 electrically connected to the second electrode 4206 may be provided. An insulating layer 4210 may be provided under the auxiliary wiring 4209.

The substrate 4201 and a sealing substrate 4211 with unevenness are bonded to each other with a sealant 4212. A barrier film 4213 and a planarization film 4214 may be provided between the sealing substrate 4211 and the light-emitting device 4202. Since the sealing substrate 4211 has the unevenness shown in FIG. 13, the extraction efficiency of light generated in the light-emitting device 4202 can be increased.

Application examples of such lighting devices include ceiling lights for indoor lighting. Examples of the ceiling lights include a ceiling direct mount light and a ceiling embedded light. Such a lighting device is fabricated using the light-emitting apparatus and a housing or a cover in combination.

As another example, such lighting devices can be used for a foot light that illuminates a floor so that safety on the floor can be improved. The foot light can be effectively used in a bedroom, on a staircase, or on a passage, for example. In such a case, the size and shape of the foot light can be changed depending on the area or structure of a room. The foot light can also be a stationary lighting device fabricated using the light-emitting apparatus and a support base in combination.

Such lighting devices can also be used for a sheet-like lighting device (sheet-like lighting). The sheet-like lighting, which is attached to a wall when used, is space-saving and thus can be used for a wide variety of applications. The area of the sheet-like lighting can be easily increased. The sheet-like lighting can also be used on a wall and a housing that have a curved surface.

Besides the above examples, the light-emitting apparatus of one embodiment of the present invention or the light-emitting device which is a part of the light-emitting apparatus can be used as part of furniture in a room, whereby a lighting device that has a function of the furniture can be obtained.

As described above, a variety of lighting devices that include the light-emitting apparatus can be obtained. Note that these lighting devices are also embodiments of the present invention.

The structures described in this embodiment can be used in an appropriate combination with any of the structures described in the other embodiments.

### Example 1

### Synthesis Example 1

In this example, a synthesis method of 2,2′,6,6′-tetraphenyl-N,N,N′,N′-tetrakis(3,5-di-tert-butylphenyl)-9,9′-bianthracene-10,10′-diamine (abbreviation: 22′66′Ph-mmtBuDPhA2BANT), which is an organic compound represented by Structural Formula (100) of Embodiment 1, is described. A structure of 22′66′Ph-mmtBuDPhA2BANT is shown below.

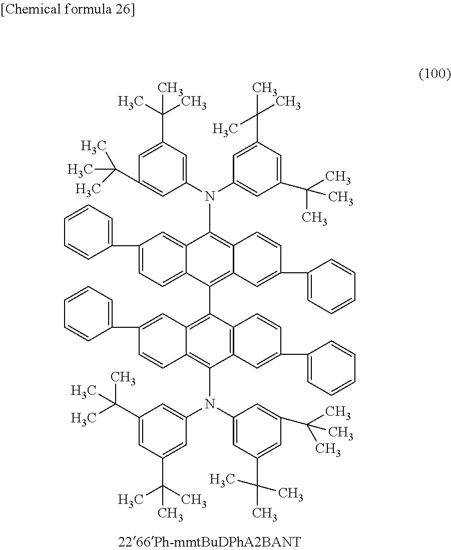 [SMILES: CC(C)(C)c1cc(N(c2cc(C(C)(C)C)cc(C(C)(C)C)c2)c2c3ccc(-c4ccccc4)cc3c(-c3c4cc(-c5ccccc5)ccc4c(N(c4cc(C(C)(C)C)cc(C(C)(C)C)c4)c4cc(C(C)(C)C)cc(C(C)(C)C)c4)c4cc(-c5ccccc5)ccc34)c3ccc(-c4ccccc4)cc23)cc(C(C)(C)C)c1]

### Step 1: Synthesis of 2,2′,6,6′-tetraphenyl-9,9′-bianthracene

Into a 500 mL three-neck flask were put 4.9 g (14 mmol) of 2,6-diphenylanthraquinone and 8.1 g (0.12 mol) of zinc, and the air in the flask was replaced with nitrogen. To this was added 20 mL of acetic acid, and the mixture was stirred at 110° C. To this was added dropwise 22 mL of concentrated hydrochloric acid, and the mixture was stirred under a nitrogen stream at 120° C. for 17 hours.

After the stirring, water was added to this mixture, and a gray solid as a residue was obtained by suction filtration. Toluene was added to this solid, the mixture was heated, and suction filtration gave a filtrate. This filtrate was concentrated, chloroform was added to the obtained yellow solid, and another suction filtration gave a filtrate. The obtained filtrate was concentrated to give a yellow solid.

The obtained solid was purified by high-performance liquid chromatography (abbreviation: HPLC) to give 1.0 g of a yellow solid in a yield of 23%. The synthesis scheme of Step 1 is shown in (a-1) below.

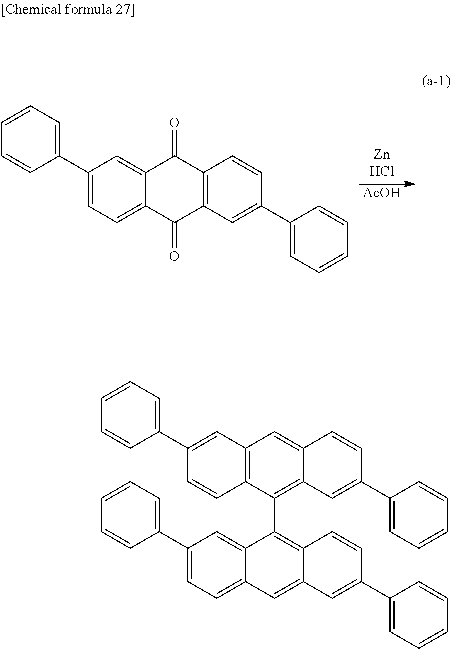

Results of ¹H NMR measurement of the yellow solid obtained in Step 1 described above are shown below. These results indicate that 2,2′,6,6′-tetraphenyl-9,9′-bianthracene was obtained.

¹H NMR (CD₂Cl₂, 300 MHz): σ=8.83 (s, 2H), 8.42 (d, J=1.8 Hz, 2H), 8.32 (d, J=8.8 Hz, 2H), 7.81-7.75 (m, 6H), 7.51-7.46 (m, 6H), 7.41-7.37 (m, 4H), 7.31-7.28 (m, 4H), 7.25-7.17 (m, 8H).

### Step 2: Synthesis of 10,10′-dibromo-2,2′,6,6′-tetraphenyl-9,9′-bianthracene

Into a 300 mL recovery flask was put 1.0 g (1.5 mmol) of 2,2′,6,6′-tetraphenyl-9,9′-bianthracene, and the air in the flask was replaced with nitrogen. To this was added 20 mL of chloroform, and the mixture was stirred at room temperature. To this solution was added 0.64 g (3.6 mmol) of N-bromosuccinimide (abbreviation: NBS), and stirring was performed under a nitrogen stream at room temperature for 15 hours.

After the stirring, water was added to this mixture, and an aqueous layer was subjected to extraction with chloroform. The obtained solution of the extract and the organic layer were combined and washed with a saturated aqueous solution of sodium thiosulfate, and then the organic layer was concentrated to give a yellow brown solid.

This solid was purified by silica gel column chromatography (developing solvent: hexane:toluene=4:1) to give 0.98 g of a target yellow solid in a yield of 78%. The synthesis scheme of Step 2 is shown in (a-2) below.

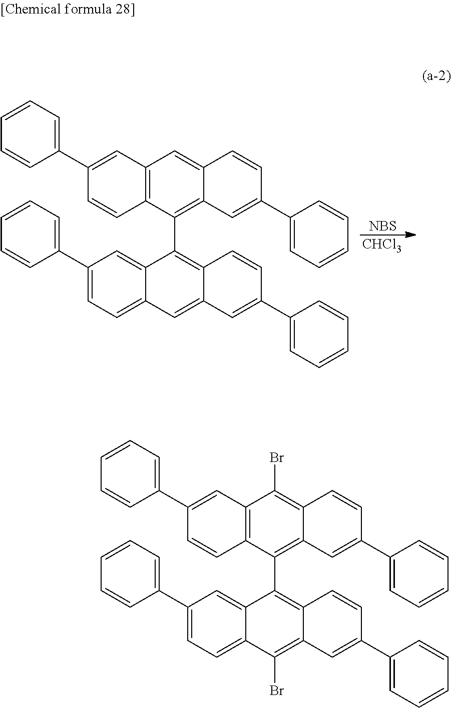

Results of ¹H NMR measurement of the yellow solid obtained in Step 2 described above are shown below. The results indicate that 10,10′-dibromo-2,2′,6,6′-tetraphenyl-9,9′-bianthracene was obtained.

¹H NMR (CD₂Cl₂, 300 MHz):σ=8.94 (d, J=1.8 Hz, 2H), 8.84 (d, J=8.8 Hz, 2H), 7.95 (dd, J=1.5 Hz, 9.2 Hz, 2H), 7.81-7.77 (m, 4H), 7.53-7.48 (m, 6H), 7.44-7.39 (m, 4H), 7.31-7.14 (m, 12H).

### Step 3: Synthesis of 22′66′Ph-mmtBuDPhA2BANT

Into a 200 mL three-neck flask were put 0.98 g (1.2 mmol) of 10,10′-dibromo-2,2′,6,6′-tetraphenyl-9,9′-bianthracene, 0.95 g (2.4 mmol) of bis(3,5-di-tert-butylphenyl)amine, 0.46 g (4.8 mmol) of sodium-t-butoxide, and 60 mg (0.15 mmol) of 2-dicyclohexylphosphino-2′,6′-dimethoxybiphenyl (abbreviation: Sphos), and the air in the flask was replaced with nitrogen. To this mixture was added 15 mL of xylene, and the mixture was degassed by being stirred under reduced pressure. To this mixture was added 40 mg (70 μmol) of bis(dibenzylideneacetone)palladium (abbreviation: Pd(dba)₂), and the mixture was stirred under a nitrogen stream at 150° C. for 2 hours.

After the stirring, 500 mL of toluene was added to the obtained mixture, which was then subjected to suction filtration through Florisil (Wako Pure Chemical Industries, Ltd., Catalog Number: 066-05265), Celite (Wako Pure Chemical Industries, Ltd., Catalog Number: 537-02305), and aluminum oxide to give a filtrate. The obtained filtrate was concentrated to give a brown solid.

This solid was purified by silica gel column chromatography (developing solvent: hexane:toluene=4:1) to give a target yellow solid. The obtained yellow solid was recrystallized with toluene and ethyl acetate to give 0.21 g of a target yellow solid in a yield of 12%. The synthesis scheme of Step 3 is shown in (a-3) below.

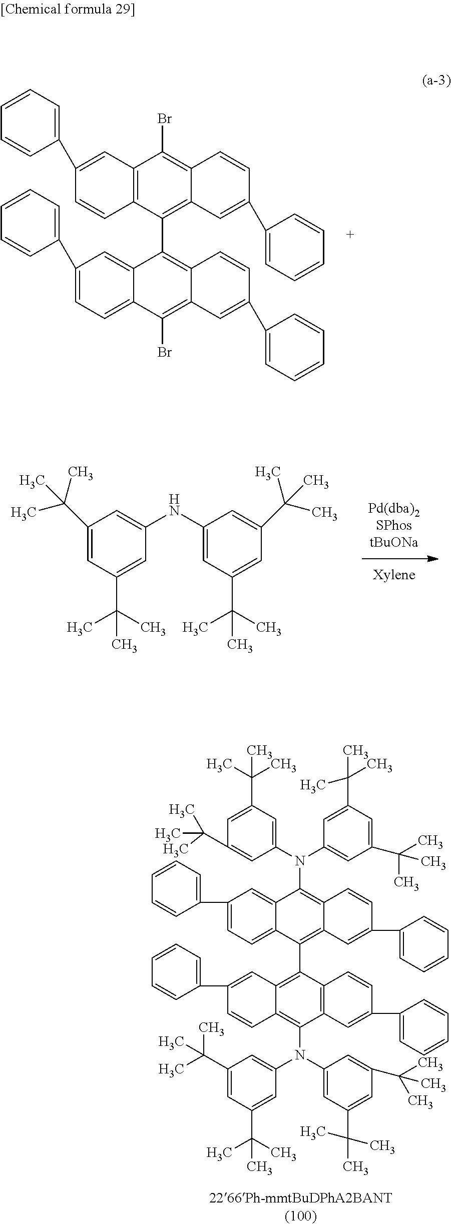 [SMILES: Brc1c2cc(-c3ccccc3)ccc2c(-c2c3ccc(-c4ccccc4)cc3c(Br)c3ccc(-c4ccccc4)cc23)c2cc(-c3ccccc3)ccc12 ; CC(C)(C)c1cc(Nc2cc(C(C)(C)C)cc(C(C)(C)C)c2)cc(C(C)(C)C)c1 ; CC(C)(C)c1cc(N(c2cc(C(C)(C)C)cc(C(C)(C)C)c2)c2c3ccc(-c4ccccc4)cc3c(-c3c4cc(-c5ccccc5)ccc4c(N(c4cc(C(C)(C)C)cc(C(C)(C)C)c4)c4cc(C(C)(C)C)cc(C(C)(C)C)c4)c4cc(-c5ccccc5)ccc34)c3ccc(-c4ccccc4)cc23)cc(C(C)(C)C)c1]

By a train sublimation method, 0.20 g of the obtained yellow solid was purified by sublimation. In the sublimation purification, the yellow solid was heated at 300° C. under a pressure of 3.0 Pa for 15 hours. After the sublimation purification, 0.17 g of a target yellow solid was obtained at a collection rate of 85%.

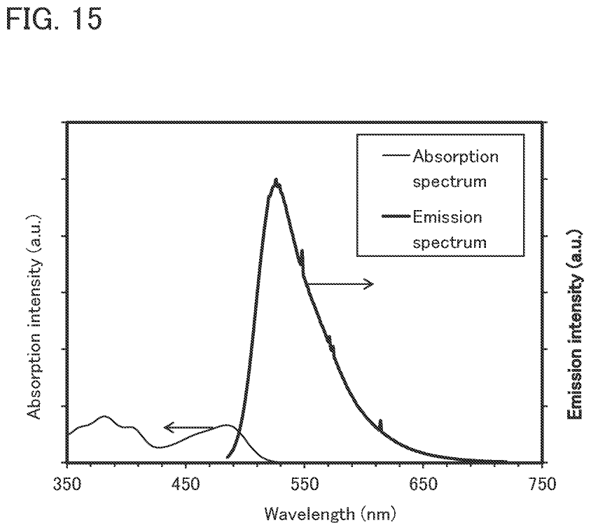

Results of ¹H NMR measurement of the yellow solid obtained in Step 3 described above will be described below. FIG. 14 shows a ¹H NMR chart. The results indicate that 22′66Ph-mmtBuDPhA2BANT (Structural Formula (100)) was obtained.

¹H NMR (CD₂Cl₂, 300 MHz):σ=8.47 (d, J=1.8 Hz, 2H), 8.44 (d, J=8.8 Hz, 2H), 7.78-7.72 (in, 4H), 7.43-7.20 (m, 26H), 7.14-7.11 (m, 6H), 7.08-7.02 (m, 4H), 1.23 (s, 36H), 1.22 (s, 36H).

Next, an absorption spectrum and an emission spectrum of a toluene solution of 22′66′Ph-mmtBuDPhA2BANT were measured. The ultraviolet-visible absorption spectrum (hereinafter simply referred to as “absorption spectrum”) and the emission spectrum were measured. The absorption spectrum was measured with an ultraviolet-visible spectrophotometer (V550DS, manufactured by JASCO Corporation). The emission spectrum was measured with a spectrofluorometer (FS920, manufactured by Hamamatsu Photonics K.K.). FIG. 15 shows the obtained measurement results of the absorption spectrum and the emission spectrum of the toluene solution. The horizontal axis represents wavelength, and the vertical axes represent absorption intensity and emission intensity.

As shown in FIG. 15, an absorption peak of 22′66′Ph-mmtBuDPhA2BANT in the toluene solution was observed at around 484 nm, and an emission wavelength peak was 526 nm (excitation wavelength: 470 nm).

### Example 2

### Synthesis Example 2

In this example, a synthesis method of 2,2′,6,6′-tetrakis(3,5-tert-butylphenyl)-N,N,N,N-tetrakis(3,5-di-tert-butylphenyl)-9,9′-bianthracene-10,10′-diamine (abbreviation: 22′66′mmtBuPh-mmtBuDPhA2BANT), which is an organic compound represented by Structural Formula (101) of Embodiment 1, is described. A structure of 22′66′mmtBuPh-mmtBuDPhA2BANT is shown below.

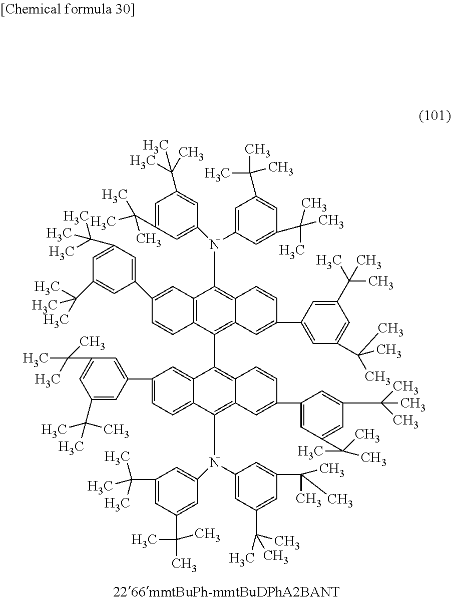 [SMILES: CC(C)(C)c1cc(-c2ccc3c(N(c4cc(C(C)(C)C)cc(C(C)(C)C)c4)c4cc(C(C)(C)C)cc(C(C)(C)C)c4)c4cc(-c5cc(C(C)(C)C)cc(C(C)(C)C)c5)ccc4c(-c4c5cc(-c6cc(C(C)(C)C)cc(C(C)(C)C)c6)ccc5c(N(c5cc(C(C)(C)C)cc(C(C)(C)C)c5)c5cc(C(C)(C)C)cc(C(C)(C)C)c5)c5cc(-c6cc(C(C)(C)C)cc(C(C)(C)C)c6)ccc45)c3c2)cc(C(C)(C)C)c1]

### Step 1: Synthesis of 2,6-bis(3,5-di-tert-butylphenyl)anthraquinone

Into a 1 L three-neck flask were put 7.48 g (20 mmol) of 2,6-dibromoanthraquinone, 13 g (42 mmol) of 2-(3,5-di-tert-butylphenyl)-4,4,5,5-tetramethyl-1,3,2-dioxaborolane, and 0.34 g (1.1 mmol) of tri(o-tolyl)phosphine (abbreviation: P(o-tol)₃), and the air in the flask was replaced with nitrogen. To this were added 200 mL of toluene, 70 mL of ethanol, and 40 mL of a 2M aqueous solution of potassium carbonate, and degassing and depressurization were performed in the flask. To the mixture was added 50 mg (0.22 mmol) of palladium(II) acetate and the mixture was stirred under a nitrogen stream at 90° C. for 9 hours.

After the stirring, water was added to this mixture, and an aqueous layer was subjected to extraction with toluene. The obtained solution of the extract and the organic layer were combined and washed with a saturated aqueous solution of sodium thiosulfate, and then the organic layer was concentrated to give a yellow brown solid.

The obtained solid was purified by silica gel column chromatography (developing solvent: hexane:toluene=1:1) to give 9.5 g of a yellow solid in a yield of 81%. The synthesis scheme of Step 1 is shown in (b-1) below.

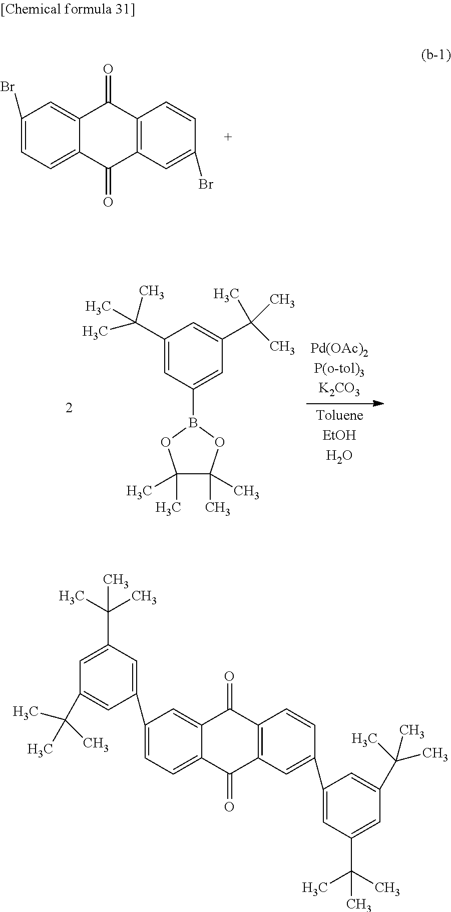 [SMILES: O=C1c2ccc(Br)cc2C(=O)c2ccc(Br)cc21 ; CC(C)(C)c1cc(B2OC(C)(C)C(C)(C)O2)cc(C(C)(C)C)c1 ; CC(C)(C)c1cc(-c2ccc3c(c2)C(=O)c2ccc(-c4cc(C(C)(C)C)cc(C(C)(C)C)c4)cc2C3=O)cc(C(C)(C)C)c1]

Results of ¹H NMR measurement of the yellow solid obtained in Step 1 described above are shown below. These results indicate that 2,6-bis(3,5-di-tert-butylphenyl)anthraquinone was obtained.

¹H NMR (CDCl₃, 300 MHz):σ=8.56 (d, J=2.0 Hz, 2H), 8.43 (d, J=8.1 Hz, 2H), 8.05 (dd, J=2.0 Hz, 8.1 Hz, 2H), 7.55 (m, 6H), 1.42 (s, 36H).

### Step 2: Synthesis of 2,2′,6,6′-tetrakis(3,5-di-tert-butylphenyl)-9,9′-bianthracene

Into a 200 mL three-neck flask were put 9.5 g (16 mmol) of 2,6-bis(3,5-di-tert-butylphenyl)anthraquinone and 22.4 g (0.34 mol) of zinc, and the air in the flask was replaced with nitrogen. To this was added 25 mL of acetic acid, and the mixture was stirred at 110° C. To this was added dropwise 53 mL of concentrated hydrochloric acid, and the mixture was stirred under a nitrogen stream at 110° C. for 43 hours. To this solution were added 20 mL of toluene and 3.2 g (49 mmol) of zinc, and the mixture was stirred at 110° C. To this was added dropwise 7.5 mL of concentrated hydrochloric acid, and the mixture was stirred under a nitrogen stream at 110° C. for 6 hours.

After the stirring, water was added to this mixture, and a gray solid as a residue was obtained by suction filtration. Chloroform was added to this gray solid, and another suction filtration gave a filtrate. The obtained filtrate was concentrated to give a yellow solid.

The obtained yellow solid was purified by high-performance liquid chromatography (abbreviation: HPLC) to give 3.4 g of a yellow solid in a yield of 37%. The synthesis scheme of Step 2 is shown in (b-2) below.

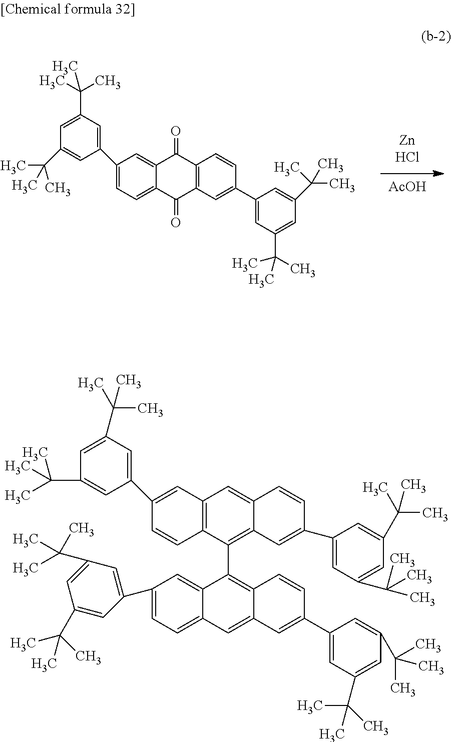

Results of ¹H NMR measurement of the yellow solid obtained in Step 2 described above are shown below. These results indicate that 2,2′,6,6′-tetrakis(3,5-di-tert-butylphenyl)-9,9′-bianthracene was obtained.

¹H NMR (CD₂Cl₂, 300 MHz):σ=8.80 (s, 2H), 8.38 (m, 2H), 8.27 (d, J=8.8 Hz, 2H), 7.78 (dd, J=1.5 Hz, 8.8 Hz, 2H), 7.59-7.54 (m, 7.49 (m, 2H), 7.45 (d, J=9.3 Hz, 2H), 7.34 (m, 2H), 7.25 (m, 2H), 7.06 (d, J=1.8 Hz, 4H), 1.40 (s, 36H), 1.13 (s, 36H).

### Step 3: Synthesis of 10,10′-dibromo-2,2′,6,6′-tetrakis(3,5-di-tert-butylphenyl)-9,9′-bianthracene

Into a 300 mL recovery flask was put 3.4 g (3.0 mmol) of 2,2′,6,6′-tetrakis(3,5-di-tert-butylphenyl)-9,9′-bianthracene, and the air in the flask was replaced with nitrogen. To this was added 30 mL of chloroform, and the mixture was stirred at room temperature. To this solution was added 1.4 g (7.9 mmol) of N-bromosuccinimide, and stirring was performed under a nitrogen stream at room temperature for 15 hours.

After the stirring, water was added to this mixture, and an aqueous layer was subjected to extraction with chloroform. The obtained solution of the extract and the organic layer were combined and washed with a saturated aqueous solution of sodium thiosulfate, and then the organic layer was concentrated to give a yellow brown solid.

This solid was purified by silica gel column chromatography (developing solvent: hexane:toluene=4:1) to give 3.5 g of a target yellow solid in a yield of 88%. The synthesis scheme of Step 3 is shown in (b-3) below.

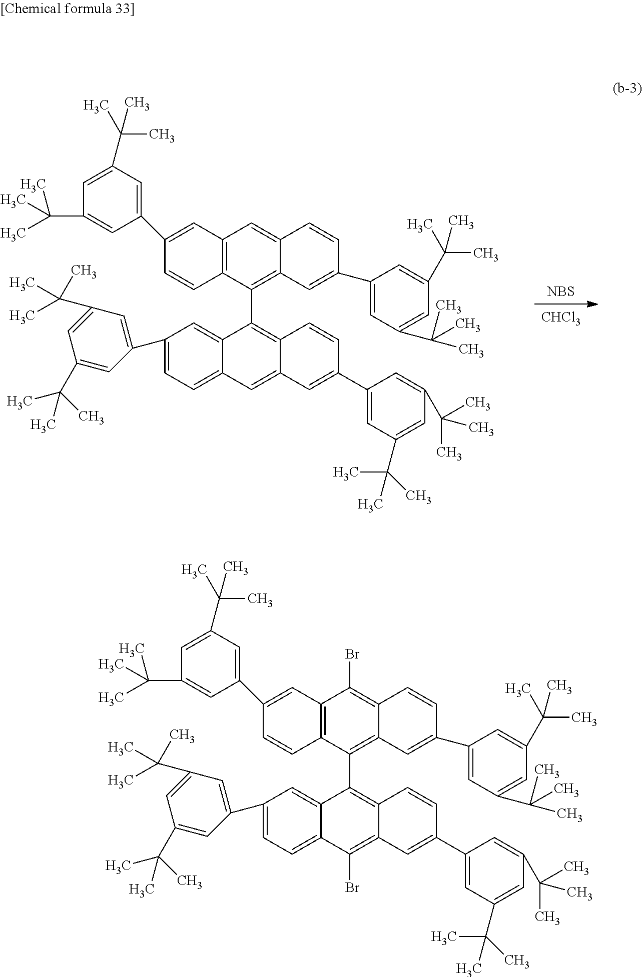

Results of ¹H NMR measurement of the yellow solid obtained in Step 3 described above will be described below. The results indicate that 10,10′-dibromo-2,2′,6,6′-tetrakis(3,5-di-tert-butylphenyl)-9,9′-bianthracene was obtained.

¹H NMR (CD₂Cl₂, 300 MHz):σ=8.90 (s, 2H), 8.78 (d, J=9.0 Hz, 2H), 7.91 (dd, J=1.8 Hz, 9.0 Hz, 2H), 7.61-7.58 (m, 6H), 7.52 (m, 2H), 7.47 (d, J=9.0 Hz, 2H), 7.31 (d, J=1.5 Hz, 2H), 7.27 (m, 2H), 7.03 (d, J=1.8 Hz, 4H), 1.41 (s, 36H), 1.13 (s, 36H).

### Step 4: Synthesis of 22′66′mmtBuPh-mmtBuDPhA2BANT

Into a 200 mL three-neck flask were put 1.2 g (0.95 mmol) of 10,10′-dibromo-2,2′,6,6′-tetrakis(3,5-di-tert-butylphenyl)-9,9′-bianthracene, 0.75 g (1.9 mmol) of bis(3,5-di-tert-butylphenyl)amine, 0.37 g (3.9 mmol) of sodium-t-butoxide, and 30 mg (73 μmol) of 2-dicyclohexylphosphino-2′,6′-dimethoxybiphenyl (abbreviation: Sphos), and the air in the flask was replaced with nitrogen. To this mixture was added 10 mL of xylene, and the mixture was degassed by being stirred under reduced pressure. To this mixture was added 20 mg (35 μmol) of bis(dibenzylideneacetone)palladium, and the mixture was stirred under a nitrogen stream at 150° C. for 4 hours.

After the stirring, 500 mL of toluene was added to the obtained mixture, which was then subjected to suction filtration through Florisil (Wako Pure Chemical Industries, Ltd., Catalog Number: 066-05265), Celite (Wako Pure Chemical Industries, Ltd., Catalog Number: 537-02305), and aluminum oxide to give a filtrate. The obtained filtrate was concentrated to give a brown solid.

This solid was purified by silica gel column chromatography (developing solvent: hexane:toluene=4:1) to give a target yellow solid. The obtained yellow solid was purified by high-performance liquid chromatography (abbreviation: HPLC) to give 50 mg of a target yellow solid in a yield of 3%. A synthesis scheme of Step 4 is shown in (b-4) below.

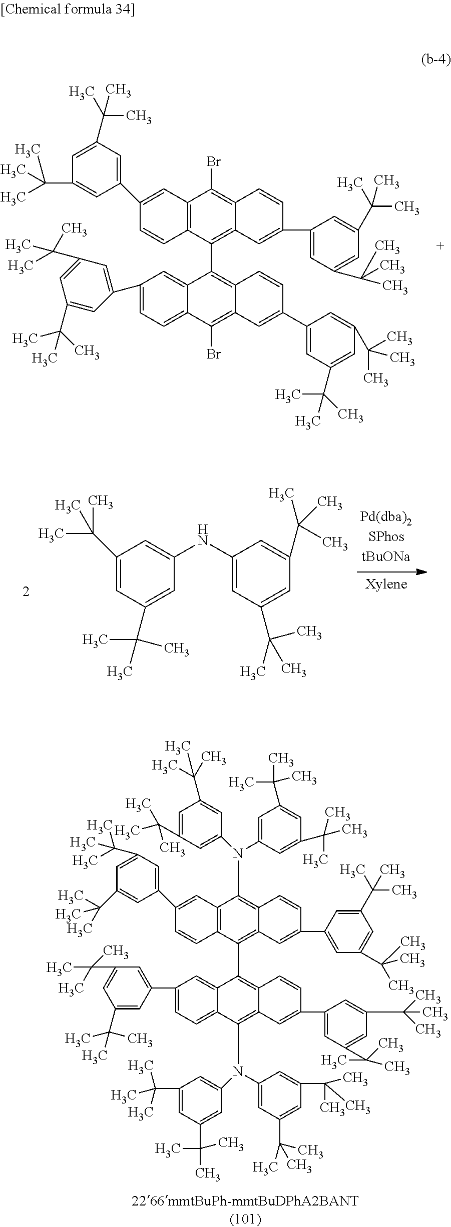

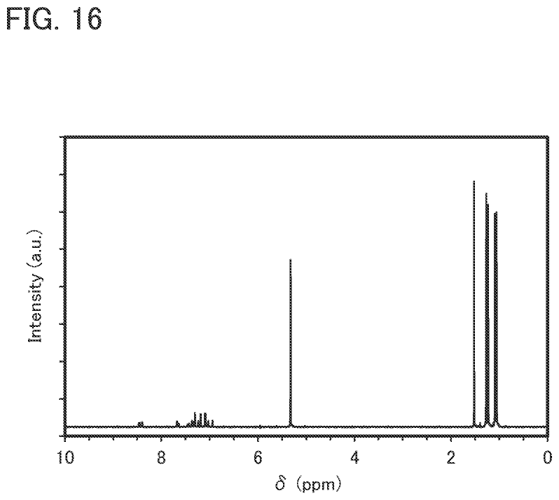

Results of ¹H NMR measurement of the yellow solid obtained in Step 4 described above will be described below. FIG. 16 shows a ¹H NMR chart. The results indicate that 22′66′mmtBuPh-mmtBuDPhA2BANT (Structural Formula (101)) was obtained.

¹H NMR (CD₂Cl₂, 300 MHz):σ=8.44 (d, J=8.7 Hz, 2H), 8.40 (m, 2H), 7.68-7.63 (m, 4H), 7.46-7.43 (m, 2H), 7.38-7.30 (m, 8H), 7.24 (m, 2H), 7.18 (d, J=1.5 Hz, 2H), 7.08 (m, 8H), 7.03 (m, 2H), 6.94 (m, 2H), 1.27 (s, 36H), 1.23 (s, 36H), 1.09 (s, 36H), 1.06 (s, 36H).

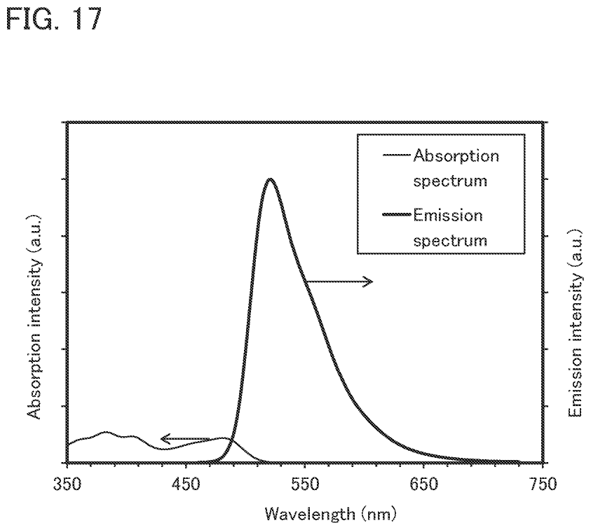

Next, an absorption spectrum and an emission spectrum of a toluene solution of 22′66′mmtBuPh-mmtBuDPhA2BANT were measured. The ultraviolet-visible absorption spectrum (hereinafter simply referred to as “absorption spectrum”) and the emission spectrum were measured. The absorption spectrum was measured with an ultraviolet-visible spectrophotometer (V550DS, manufactured by JASCO Corporation). The emission spectrum was measured with a spectrofluorometer (FP-8600, manufactured by JASCO Corporation). FIG. 17 shows the obtained measurement results of the absorption spectrum and the emission spectrum of the toluene solution. The horizontal axis represents wavelength, and the vertical axes represent absorption intensity and emission intensity.

As shown in FIG. 17, an absorption peak of 22′66′mmtBuPh-mmtBuDPhA2BANT in the toluene solution was observed at around 481 nm, and an emission wavelength peak was 521 nm (excitation wavelength: 450 nm).

### Example 3

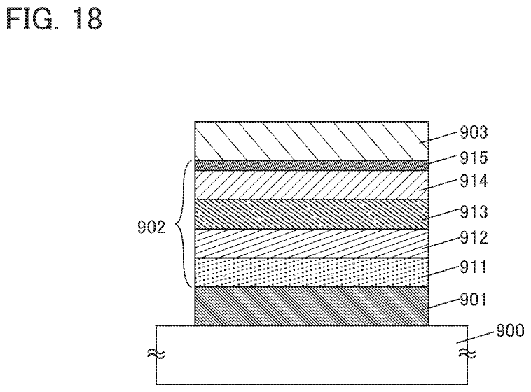

In this example, light-emitting devices were fabricated using the compound of one embodiment of the present invention, and the operation characteristics were measured. The light-emitting devices described in this example are Light-emitting device 1-1, Light-emitting device 1-2, Light-emitting device 1-3, Comparative light-emitting device 1-a, and Comparative light-emitting device 1-b. These light-emitting devices each have an element structure illustrated in FIG. 18. A light-emitting layer 913 of this example has a structure described in Structure example of light-emitting layer in Embodiment 2, which is specifically the structure shown in Table 1. In Light-emitting device 1-1, Light-emitting device 1-2, and Light-emitting device 1-3, the light-emitting layers of the light-emitting devices each include the compound of one embodiment of the present invention, 2,2′,6,6′,-tetraphenyl-N,N,N′,N′-tetrakis(3,5-di-tert-butylphenyl)-9,9′-bianthracene-10,10′-diamine (abbreviation: 22′66′Ph-mmtBuDPhA2BANT), in addition to 9-[3-(4,6-diphenyl-1,3,5-triazin-2-yl)phenyl]-9′-phenyl-2,3′-bi-9H-carbazole (abbreviation: mPCCzPTzn-02), 3,3′-bis(9-phenyl-9H-carbazole) (abbreviation: PCCP), and [2-(4-methyl-5-phenyl-2-pyridinyl-κN)phenyl-κC]bis[2-(2-pyridinyl-κN)phenyl-κC]iridium (abbreviation: [Ir(ppy)₂(mdppy)]), and have different amounts of 22′66′Ph-mmtBuDPhA2BANT. Comparative light-emitting device 1-a described as a comparative example is a light-emitting device using N10,N10,N10′,N10′-tetra-tolyll-9,9′-bianthracene-10,10′-diamine (abbreviation: BA-TTB), instead of 22′66′Ph-mmtBuDPhA2BANT used in the light-emitting layers of Light-emitting device 1-1 and Light-emitting device 1-2. Comparative light-emitting device 1-b is a light-emitting device in which the light-emitting layer includes only mPCCzPTzn-02, PCCP, and [Ir(ppy)₂(mdppy)]. Chemical formulae of materials used in this example are shown below.

TABLE 1

| | | Hole- | Hole- | Light- | | | Electron- | |
|---|---|---|---|---|---|---|---|---|
| | First | injection | transport | emitting | Electron- | | injection | Second |
| | electrode | layer | layer | layer | transport layer | | layer | electrode |
| | 901 | 911 | 912 | 913 | 914 | | 915 | 903 |
| Light-emitting | ITSO | DBT3P-II:MoOx | PCBBi1BP | * | mPCCzPTzn-02 | NBphen | LiF | Al |
| device 1-1 | (70 nm) | (1:0.5 40 nm) | (20 nm) | | (20 nm) | (10 nm) | (1 nm) | (200 nm) |
| Light-emitting | | | | ** | | | | |
| device 1-2 | | | | | | | | |
| Light-emitting | | | | *** | | | | |
| device 1-3 | | | | | | | | |
| Comparative | | | | **** | | | | |
| light-emitting | | | | | | | | |
| evice 1-a | | | | | | | | |
| Comparative | | | | ***** | | | | |
| light-emitting | | | | | | | | |
| device 1-b | | | | | | | | |

* mPCCzPTzn-02:PCCP:[Ir(ppy)₂(mdppy)]:22′66′Ph-mmtBuDPhA2BANT (0.5:0.5:0.1:0.01 40 nm)
** mPCCzPTzn-02:PCCP:[Ir(ppy)₂(mdppy)]:22′66′Ph-mmtBuDPhA2BANT (0.5:0.5:0.1:0.025 40 nm)
*** mPCCzPTzn-02:PCCP:[Ir(ppy)₂(mdppy)]:22′66′Ph-mmtBuDPhA2BANT (0.5:0.5:0.1:0.05 40 nm)
**** mPCCzPTzn-02:PCCP:[Ir(ppy)₂(mdppy)]:BA-TTB (0.5:0.5:0.1:0.05 40 nm)
***** mPCCzPTzn-02:PCCP:[Ir(ppy)₂(mdppy)] (0.5:0.5:0.1 40 nm)

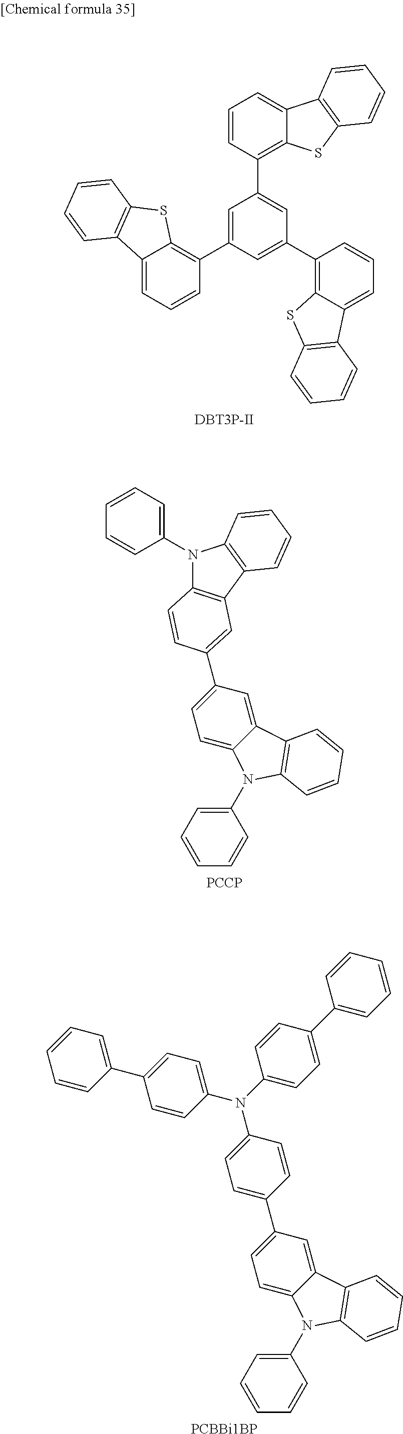

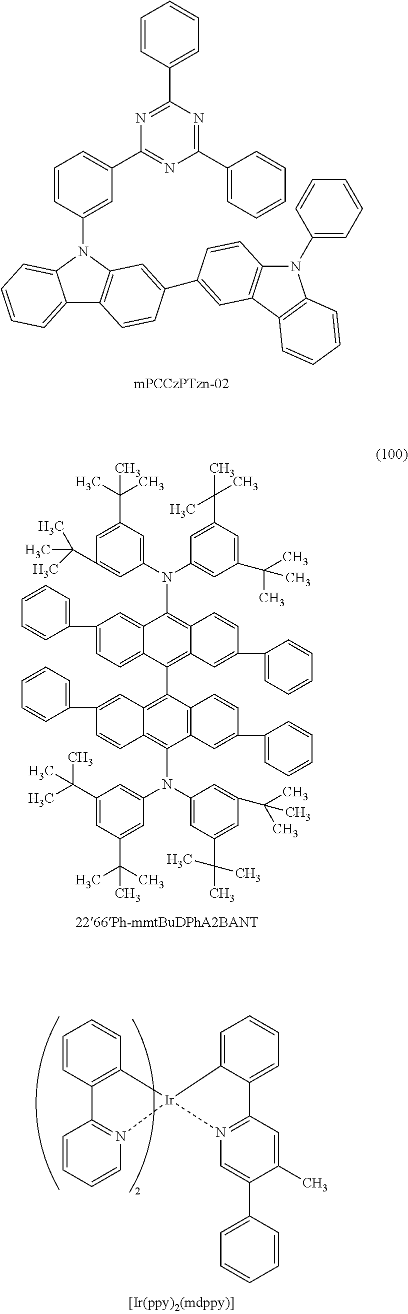

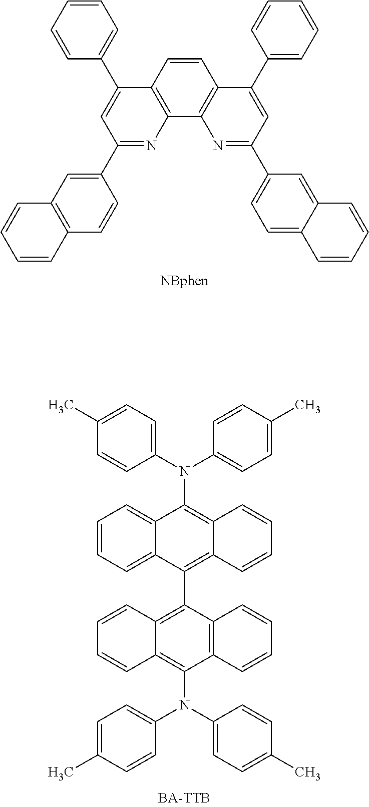

A glass substrate was used as the substrate 900. As the first electrode 901, a film of indium tin oxide containing silicon oxide (ITSO) was used and the thickness was set to 70 nm. Note that the electrode area of the first electrode 901 is 4 mm² (2 mm×2 mm).

The hole-injection layer 911 was a film formed by co-evaporation of 4,4′,4″-(benzene-1,3,5-triyl)tri(dibenzothiophene) (abbreviation: DBT3P-II) and molybdenum oxide (DBT3P-II:molybdenum oxide=1:0.5 (mass ratio)), and the thickness was set to 40 nm.

For the hole-transport layer 912, 4,4′-diphenyl-4″-(9-phenyl-9H-carbazol-3-yl)triphenylamine (abbreviation: PCBBi1BP) was used, and the thickness was set to 20 nm.

In each of Light-emitting device 1-1, Light-emitting device 1-2, and Light-emitting device 1-3, the light-emitting layer 913 includes a film including 9-[3-(4,6-diphenyl-1,3,5-triazin-2-yl)phenyl]-9′-phenyl-2,3′-bi-9H-carbazole (abbreviation: mPCCzPTzn-02), 3,3′-bis(9-phenyl-9H-carbazole) (abbreviation: PCCP), [2-(4-methyl-5-phenyl-2-pyridinyl-κN)phenyl-κC]bis[2-(2-pyridinyl-κN)phenyl-κC]iridium (abbreviation: [Ir(ppy)₂(mdppy)]), and 2,2′,6,6′,-tetraphenyl-N,N,N′,N′-tetrakis(3,5-di-tert-butylphenyl)-9,9′-bianthracene-10,10′-diamine (abbreviation: 22′66′Ph-mmtBuDPhA2BANT), and the thickness was set to 40 nm. As the light-emitting layer 913 in Comparative light-emitting device 1-a, a film including mPCCzPTzn-02, PCCP, [Ir(ppy)₂(mdppy)], and BA-TTB was used and the thickness was set to 40 nm. As the light-emitting layer 913 in Comparative light-emitting device 1-b, a film including mPCCzPTzn-02, PCCP, and [Ir(ppy)₂(mdppy)] was used and the thickness was set to 40 nm. Note that the weight ratios in the light-emitting layers 913 of the light-emitting devices, which are different from one another, are shown in Table 1.

As the electron-transport layer 914, a stacked film of 20-nm-thick mPCCzPTzn-02 and 10-nm-thick 2,9-bis(naphthalen-2-yl)-4,7-diphenyl-1,10-phenanthroline (abbreviation: NBphen) was used.

For the electron-injection layer 915, lithium fluoride (LiF) was used and the thickness was set to 1 nm.

For the second electrode 903, aluminum was used and the thickness was set to 200 nm. In this example, the second electrode 903 functions as a cathode.

### <<Operation Characteristics of Light-Emitting Devices>>

Operation characteristics of the fabricated light-emitting devices were measured. Luminance and chromaticity (CIE chromaticity) were measured with a luminance colorimeter (BM-5A manufactured by TOPCON TECHNOHOUSE CORPORATION), and electroluminescence (EL) spectra were measured with a multi-channel spectrometer (PMA-11 manufactured by Hamamatsu Photonics K.K.). Note that the measurement was carried out at room temperature (an atmosphere maintained at 23° C.).

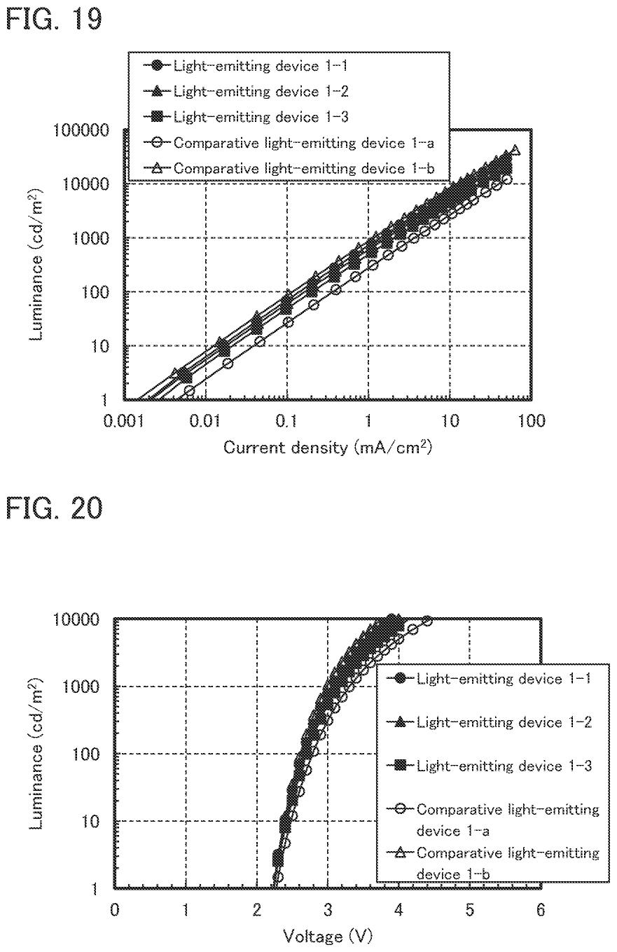

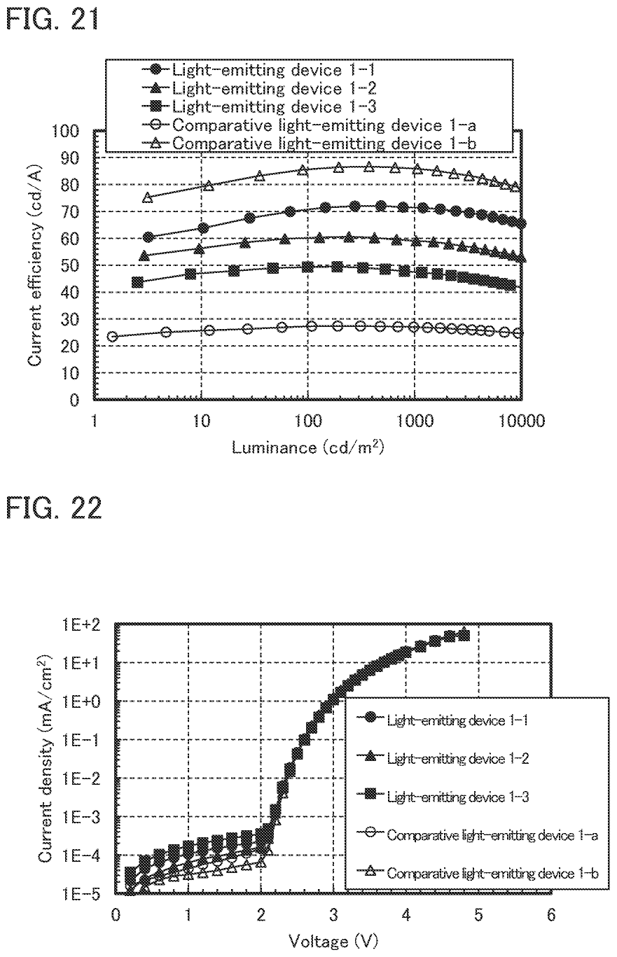

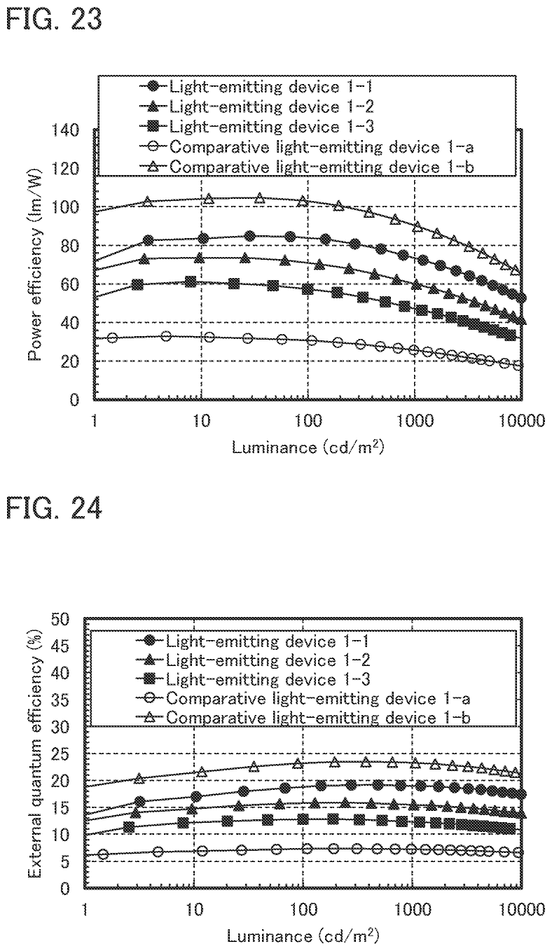

As the results of the operation characteristics of Light-emitting device 1-1, Light-emitting device 1-2, Light-emitting device 1-3, Comparative light-emitting device 1-a, and Comparative light-emitting device 1-b fabricated in this example, FIG. 19 shows current density-luminance characteristics, FIG. 20 shows voltage-luminance characteristics, FIG. 21 shows luminance-current efficiency characteristics, FIG. 22 shows voltage-current density characteristics, FIG. 23 shows luminance-power efficiency characteristics, and FIG. 24 shows luminance-external quantum efficiency characteristics.

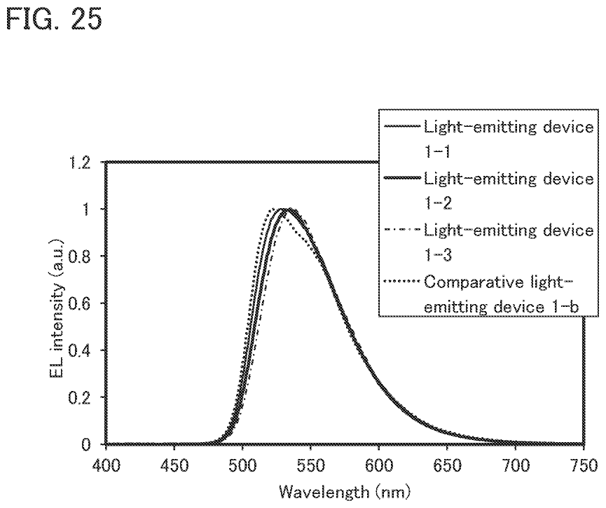

FIG. 25 shows the electroluminescence spectra (EL spectra) of the light-emitting devices to which current flows at a current density of 2.5 mA/cm².

Next, Table 2 below shows the initial values of the main characteristics of the light-emitting devices at around 1000 cd/m².

TABLE 2

| | | | | | | | | | External |
|---|---|---|---|---|---|---|---|---|---|
| | | | Current | | | | Current | Power | quantum |
| | Voltage | Current | density | Chromaticity | | Luminance | efficiency | efficiency | efficiency |
| | (V) | (mA) | (mA/cm²) | x | y | (cd/m²) | (cd/A) | (lm/W) | (%) |
| Light-emitting | 3.1 | 0.067 | 1.7 | 0.33 | 0.64 | 1200 | 71 | 72 | 19 |
| device 1-1 | | | | | | | | | |
| Light-emitting | 3.1 | 0.070 | 1.8 | 0.34 | 0.64 | 1000 | 59 | 60 | 15 |
| device 1-2 | | | | | | | | | |
| Light-emitting | 3.2 | 0.099 | 2.5 | 0.34 | 0.64 | 1200 | 47 | 46 | 12 |
| device 1-3 | | | | | | | | | |
| Comparative | 3.3 | 0.15 | 3.6 | 0.33 | 0.64 | 980 | 27 | 26 | 7 |
| light-emitting | | | | | | | | | |
| device 1-a | | | | | | | | | |
| Comparative | 3.0 | 0.050 | 1.2 | 0.32 | 0.65 | 1100 | 86 | 90 | 23 |
| light-emitting | | | | | | | | | |
| device 1-b | | | | | | | | | |

Light-emitting device 1-1, Device 1-2, and Light-emitting device 1-3 are each an element in which the compound of one embodiment of the present invention, 22′66′Ph-mmtBuDPhA2BANT, is added to the light-emitting layer of Comparative light-emitting device 1-b. As shown in FIG. 25, the EL spectrum of Comparative light-emitting device 1-b exhibited green light emission with a peak wavelength of 522 nm which originates from a phosphorescent substance [Ir(ppy)₂(mdppy)]. The EL spectra of Light-emitting device 1-1 to Light-emitting device 1-3 exhibited green light emission with a peak wavelength of around 530 nm which originates from 22′66′Ph-mmtBuDPhA2BANT. This indicates that in Light-emitting device 1-1 to Light-emitting device 1-3, 22′66′Ph-mmtBuDPhA2BANT, which is a fluorescent substance, receives excitation energy and emits light. The above results show that Light-emitting device 1-1 to Light-emitting device 1-3 each have a high external quantum efficiency of 12% or higher. The generation probability of singlet excitons which are generated by recombination of carriers (holes and electrons) injected from the pair of electrodes is at most 25%; therefore, the external quantum efficiency of a fluorescent light-emitting device in the case where the light extraction efficiency to the outside is 30% is at most 7.5%. However, an external quantum efficiency higher than 7.5% was obtained in Light-emitting device 1-1 to Light-emitting device 1-3. This is because, in addition to light emission derived from singlet excitons generated by recombination of carriers (holes and electrons) injected from the pair of electrodes, light emission derived from energy transfer from triplet excitons is obtained from the fluorescent substance.

Comparison between Light-emitting device 1-1 to Light-emitting device 1-3, which have different concentrations of 22′66′Ph-mmtBuDPhA2BANT in the light-emitting layers, shows that all the light-emitting devices have high external quantum efficiency. Comparative light-emitting device 1-a using BA-TTB shows a lower external quantum efficiency than Light-emitting device 1-3 though these light-emitting devices have the same concentration ratio of the fluorescent substances. Thus, it was found that the compound of one embodiment of the present invention, 22′66′Ph-mmtBuDPhA2BANT, inhibited deactivation of triplet excitation energy, which becomes problematic particularly when the concentration is high, and efficiently emitted light in the light-emitting layers of the light-emitting devices.

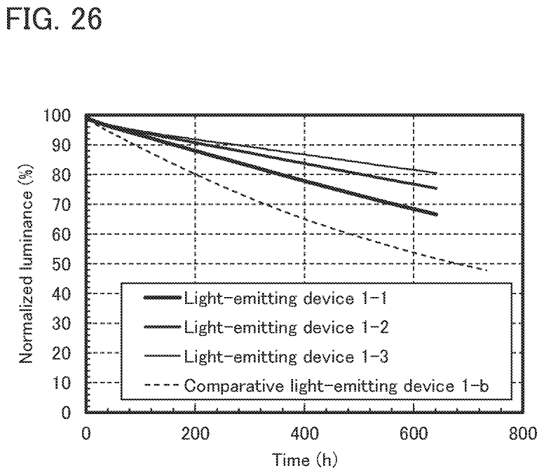

Driving tests of Light-emitting device 1-1, Light-emitting device 1-2, Light-emitting device 1-3, and Comparative light-emitting device 1-b at a constant current density of 50 mA/cm² were performed. The results are shown in FIG. 26. The results show that an increase in the concentration of 22′66′Ph-mmtBuDPhA2BANT improves the reliability while high emission efficiency is achieved. This means that by increasing the concentration of a guest in the light-emitting layer, excitation energy in the light-emitting layer can be efficiently converted into light emission of the guest and non-radiative deactivation can be inhibited. In other words, it is suggested that an increase in the concentration of the guest can prevent energy transfer from the host to the guest due to the Dexter mechanism and can increase the rate of the energy transfer from the host to the guest due to the Förster mechanism. Therefore, the light-emitting device including the compound of one embodiment of the present invention has high emission efficiency and high reliability.

### Example 4

In this example, light-emitting devices were fabricated using the compound of one embodiment of the present invention, and the operation characteristics were measured. The light-emitting devices described in this example are Light-emitting device 2-1, Light-emitting device 2-2, Light-emitting device 2-3, Light-emitting device 2-4, Comparative light-emitting device 2-a, and Comparative light-emitting device 2-b. Each of these light-emitting devices has an element structure illustrated in FIG. 18. The EL layer 902 of this example has a structure described in Structure example 3 of light-emitting layer in Embodiment 2, which is specifically the structure shown in Table 3. In Light-emitting device 2-1, Light-emitting device 2-2, Light-emitting device 2-3, and Light-emitting device 2-4, the light-emitting layers of the light-emitting devices each include the compound of one embodiment of the present invention, 2,2′,6,6′,-tetraphenyl-N,N,N′,N′-tetrakis(3,5-di-tert-butylphenyl)-9,9′-bianthracene-10,10′-diamine (abbreviation: 22′66′Ph-mmtBuDPhA2BANT), in addition to 4,6-bis[3-(9H-carbazol-9-yl)phenyl]pyrimidine (abbreviation: 4,6mCzP2Pm) and tris[2-(1H-pyrazol-1-yl-κN2)phenyl-κC]iridium(III) (abbreviation: [Ir(ppz)₃]), and have different amounts of 22′66′Ph-mmtBuDPhA2BANT. Comparative light-emitting device 2-a described as a comparative example is a light-emitting device using N10,N10,N10′,N10′-tetra-tolyll-9,9′-bianthracene-10,10′-diamine (abbreviation: BA-TTB) instead of 22′66′Ph-mmtBuDPhA2BANT used in the light-emitting layers of Light-emitting device 2-1, Light-emitting device 2-2, Light-emitting device 2-3, and Light-emitting device 24. Comparative light-emitting device 1-b is a light-emitting device in which the light-emitting layer includes only 4,6mCzP2Pm and [Ir(ppz)₃]. Chemical formulae of materials used in this example are shown below.

TABLE 3

| | | Hole- | Hole- | Light- | | | Electron- | |
|---|---|---|---|---|---|---|---|---|
| | First | injection | transport | emitting | Electron- | | injection | Second |
| | electrode | layer | layer | layer | transport layer | | layer | electrode |
| | 901 | 911 | 912 | 913 | 914 | | 915 | 903 |
| Light-emitting | ITSO | DBT3P-II:MoOx | PCCP | * | 4,6mCzP2Pm | NBphen | LiF | Al |
| device 2-1 | (70 nm) | (1:0.5 40 nm) | (20 nm) | | (20 nm) | (10 nm) | (1 nm) | (200 nm) |
| Light- emitting | | | | ** | | | | |
| device 2-2 | | | | | | | | |
| Light- emitting | | | | *** | | | | |
| device 2-3 | | | | | | | | |
| Light-emitting | | | | **** | | | | |
| device 2-4 | | | | | | | | |
| Comparative | | | | ***** | | | | |
| light-emitting | | | | | | | | |
| device 2-a | | | | | | | | |
| Comparative | | | | ****** | | | | |
| light-emitting | | | | | | | | |
| device 2-b | | | | | | | | |

* 4,6mCzP2Pm:[Ir(ppz)₃]:22′66′Ph-mmtBuDPhA2BANT (0.8:0.2:0.01 40 nm)
** 4,6mCzP2Pm:[Ir(ppz)₃]:22′66′Ph-mmtBuDPhA2BANT (0.8:0.2:0.025 40 nm)
*** 4,6mCzP2Pm:[Ir(ppz)₃]:22′66′Ph-mmtBuDPhA2BANT (0.8:0.2:0.05 40 nm)
**** 4,6mCzP2Pm:[Ir(ppz)₃]:22′66′Ph-mmtBuDPhA2BANT (0.8:0.2:0.1 40 nm)

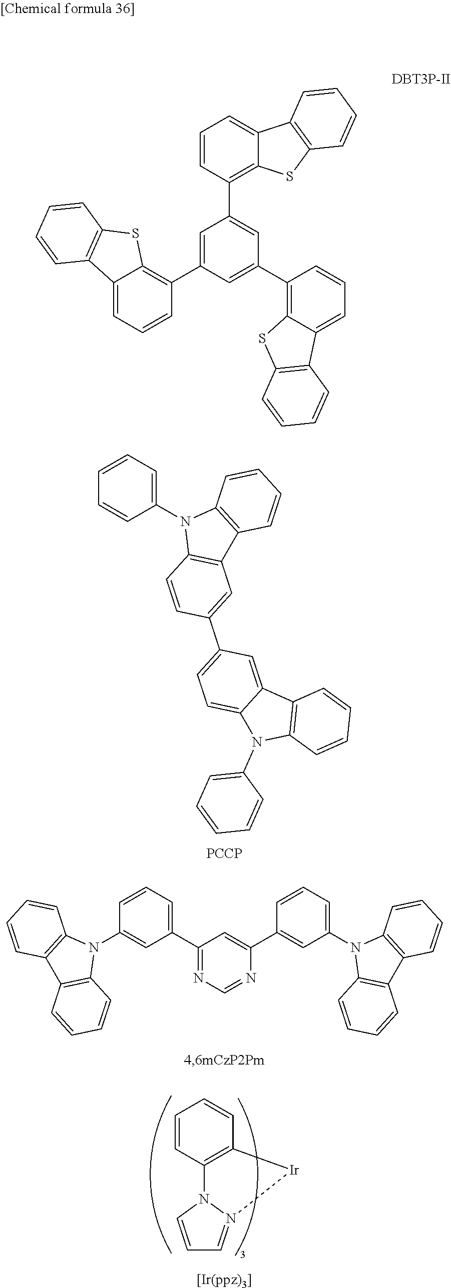

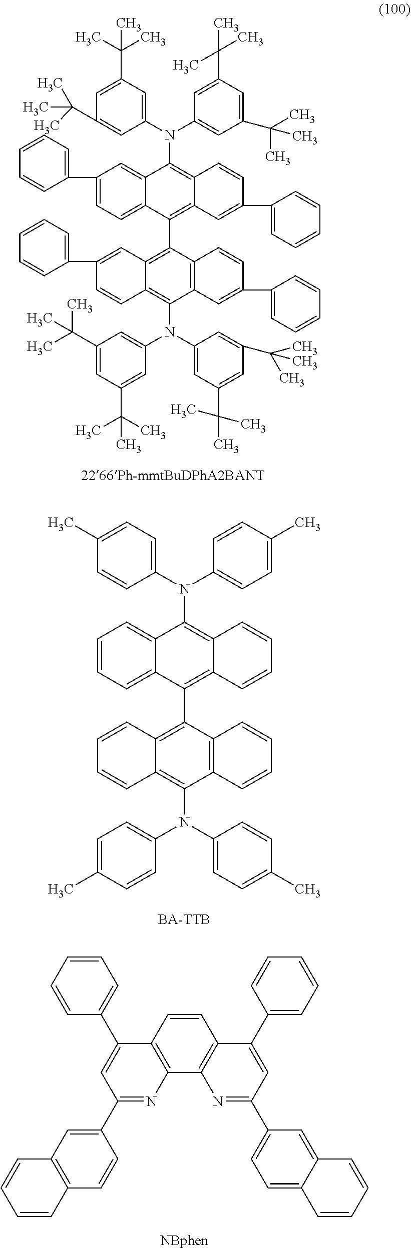

### <<Structure of Light-Emitting Devices>>

The light-emitting devices described in this example have a structure shown in FIG. 16, in which a hole-injection layer 911, a hole-transport layer 912, a light-emitting layer 913, an electron-transport layer 914, and an electron-injection layer 915 included in an EL layer 902 are stacked in this order over a first electrode 901 formed over a substrate 900, and a second electrode 903 is stacked over the electron-injection layer 915.

A glass substrate was used as the substrate 900. As the first electrode 901, a film of indium tin oxide containing silicon oxide (ITSO) was used and the thickness was set to 70 nm. Note that the electrode area of the first electrode 901 is 4 mm² (2 mm×2 mm).

The hole-injection layer 911 was a film formed by co-evaporation of 4,4′,4″-(benzene-1,3,5-triyl)tri(dibenzothiophene) (abbreviation: DBT3P-II) and molybdenum oxide (DBT3P-II:molybdenum oxide=1:0.5 (mass ratio)), and the thickness was set to 40 nm.

For the hole-transport layer 912, 3,3′-bis(9-phenyl-9H-carbazole) (abbreviation: PCCP) was used and the thickness was set to 20 nm.

For the light-emitting layer 913 of each of Light-emitting device 2-1, Light-emitting device 2-2, Light-emitting device 2-3, and Light-emitting device 2-4, a film including 4,6mCzP2Pm, [Ir(ppz)₃], and 22′66′Ph-mmtBuDPhA2BANT was used, and the thickness was set to 40 nm. For the light-emitting layer 913 of Comparative light-emitting device 2-a, a film including 4,6mCzP2Pm, [Ir(ppz)₃], and BA-TTB was used, and the thickness was set to 40 nm. For the light-emitting layer 913 of Comparative light-emitting device 2-b, a film including 4,6mCzP2Pm and [Ir(ppz)₃] was used, and the thickness was set to 40 nm. Note that the weight ratios in the light-emitting layers 913 of the light-emitting devices, which are different from one another, are shown in Table 3.

As the electron-transport layer 914, a stacked film of 20-nm-thick 4,6mCzP2Pm and 10-nm-thick NBphen was used.

For the electron-injection layer 915, lithium fluoride (LiF) was used and the thickness was set to 1 nm.

For the second electrode 903, aluminum was used and the thickness was set to 200 nm. In this example, the second electrode 903 functions as a cathode.

### <<Operation Characteristics of Light-Emitting Devices>>

Operation characteristics of the fabricated light-emitting devices were measured. Luminance and chromaticity (CIE chromaticity) were measured with a luminance colorimeter (BM-5A manufactured by TOPCON TECHNOHOUSE CORPORATION), and electroluminescence (EL) spectra were measured with a multi-channel spectrometer (PMA-11 manufactured by Hamamatsu Photonics K.K.). Note that the measurement was carried out at room temperature (an atmosphere maintained at 23° C.).

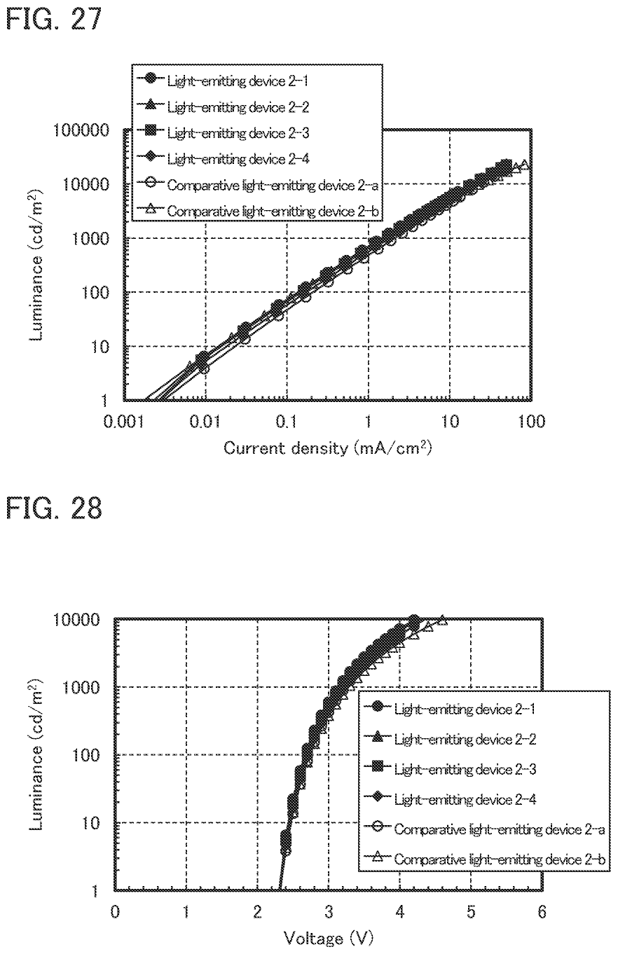

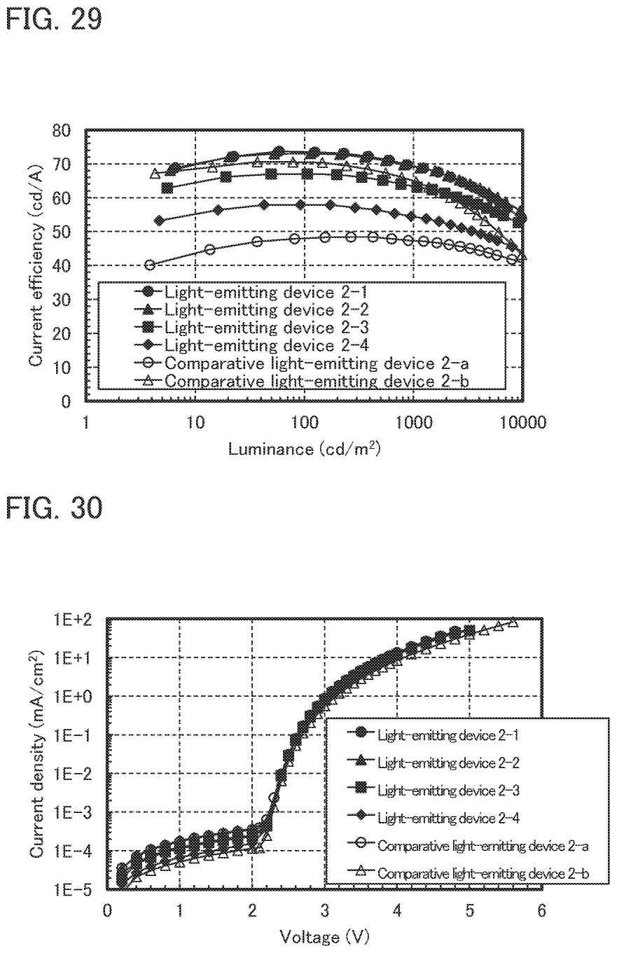

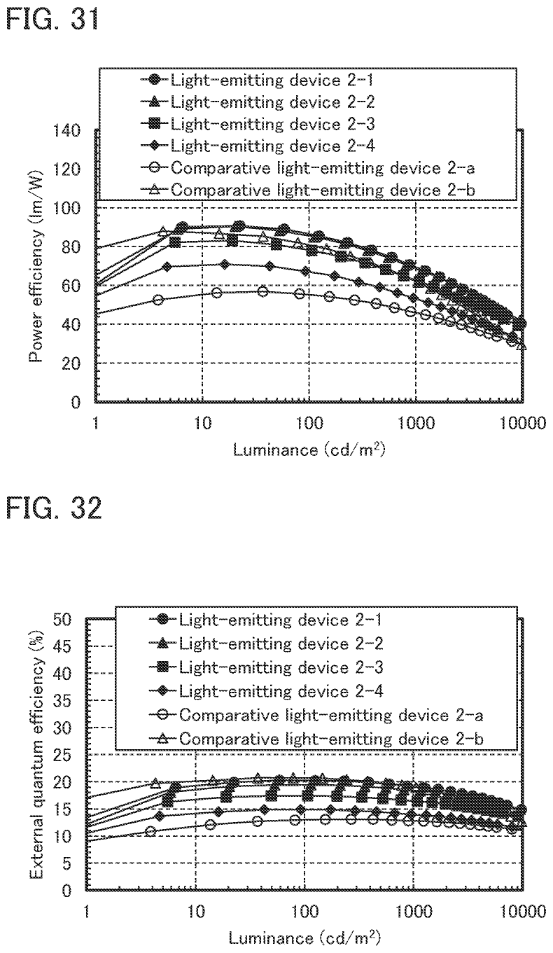

As the results of the operation characteristics of Light-emitting device 2-1, Light-emitting device 2-2, Light-emitting device 2-3, Light-emitting device 2-4, Comparative light-emitting device 2-a, and Comparative light-emitting device 2-b fabricated in this example, the current density-luminance characteristics are shown in FIG. 27, the voltage-luminance characteristics are shown in FIG. 28, the luminance-current efficiency characteristics are shown in FIG. 29, the voltage-current density characteristics are shown in FIG. 30, the luminance-power efficiency characteristics are shown in FIG. 31, and the luminance-external quantum efficiency characteristics are shown in FIG. 32.

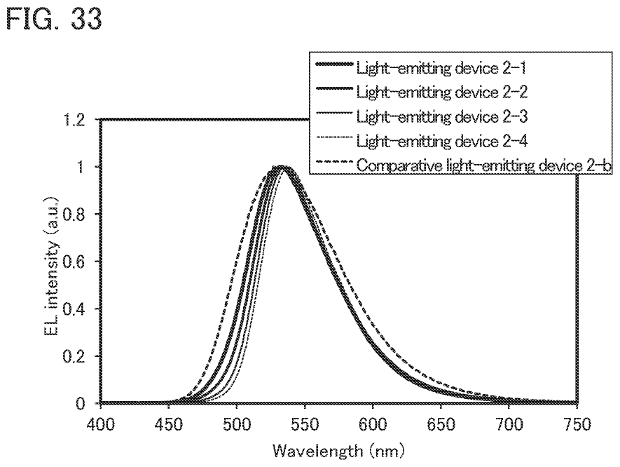

FIG. 33 shows the electroluminescence spectra (EL spectra) of the light-emitting devices to which current flows at a current density of 2.5 mA/cm².

Table 4 below shows the initial values of the main characteristics of each of the light-emitting devices at around 1000 cd/m².

TABLE 4

| | | | | | | | | | External |
|---|---|---|---|---|---|---|---|---|---|
| | | | Current | | | | Current | Power | quantum |
| | Voltage | Current | density | Chromaticity | | Luminance | efficiency | efficiency | efficiency |
| | (V) | (mA) | (mA/cm²) | x | y | (cd/m²) | (cd/A) | (lm/W) | (%) |
| Light- | 3.1 | 0.050 | 1.3 | 0.32 | 0.63 | 880 | 70 | 71 | 19 |
| emitting | | | | | | | | | |
| device 2-1 | | | | | | | | | |
| Light- | 3.2 | 0.067 | 1.7 | 0.33 | 0.64 | 1100 | 69 | 67 | 18 |
| emitting | | | | | | | | | |
| device 2-2 | | | | | | | | | |
| Light- | 3.2 | 0.068 | 1.7 | 0.33 | 0.64 | 1100 | 63 | 62 | 16 |
| emitting | | | | | | | | | |
| device 2-3 | | | | | | | | | |
| Light- | 3.2 | 0.070 | 1.7 | 0.34 | 0.64 | 1000 | 55 | 54 | 14 |
| emitting | | | | | | | | | |
| device 2-4 | | | | | | | | | |
| Comparative | 3.2 | 0.075 | 1.9 | 0.32 | 0.64 | 900 | 47 | 47 | 13 |
| light-emitting | | | | | | | | | |
| device 2-a | | | | | | | | | |
| Comparative | 3.3 | 0.065 | 1.6 | 0.32 | 0.61 | 1100 | 65 | 62 | 19 |
| light-emitting | | | | | | | | | |
| device 2-b | | | | | | | | | |

Light-emitting device 2-1, Light-emitting device 2-2, Light-emitting device 2-3, and Light-emitting device 2-4 are elements in which the compound of one embodiment of the present invention, 22′66′Ph-mmtBuDPhA2BANT, is added to the light-emitting layer of Comparative light-emitting device 2-b. As shown in FIG. 33, the EL spectrum of Comparative light-emitting device 2-b exhibited green light emission with a peak wavelength of 531 nm which originates from an exciplex of 4,6mCzP2Pm and [Ir(ppz)₃], which is different from an emission spectrum exhibited by each of 4,6mCzP2Pm and [Ir(ppz)₃]. The EL spectra of Light-emitting device 2-1 to Light-emitting device 2-4 exhibited green light emission with a peak wavelength of around 530 nm which originates from 22′66′Ph-mmtBuDPhA2BANT. This indicates that in Light-emitting device 2-1 to Light-emitting device 2-4, 22′66′Ph-mmtBuDPhA2BANT, which is a fluorescent substance, receives excitation energy and emits light. The above results show that Light-emitting device 2-1 to Light-emitting device 2-4 each have a high external quantum efficiency of 14% or higher. The generation probability of singlet excitons which are generated by recombination of carriers (holes and electrons) injected from the pair of electrodes is at most 25%; therefore, the external quantum efficiency of a fluorescent light-emitting device in the case where the light extraction efficiency to the outside is 30% is at most 7.5%. However, an external quantum efficiency higher than 7.5% was obtained in Light-emitting device 2-1 to Light-emitting device 2-4. This is because, in addition to light emission derived from singlet excitons generated by recombination of carriers (holes and electrons) injected from the pair of electrodes, light emission derived from energy transfer from triplet excitons is obtained from the fluorescent substance.

Comparison between Light-emitting device 2-1 to Light-emitting device 2-4, which have different concentrations of 22′66′Ph-mmtBuDPhA2BANT in the light-emitting layers, shows that all the light-emitting devices have high external quantum efficiency. Comparison between Comparative light-emitting device 2-a using BA-TTB and Light-emitting device 2-3, which have the same concentration ratio of the fluorescent substances, shows that the external quantum efficiency of Comparative light-emitting device 2-a is lower than that of Light-emitting device 2-3. Thus, it was found that the compound of one embodiment of the present invention, 22′66′Ph-mmtBuDPhA2BANT, inhibited deactivation of triplet excitation energy, which becomes problematic particularly when the concentration is high, and efficiently emitted light in the light-emitting layers of the light-emitting devices.

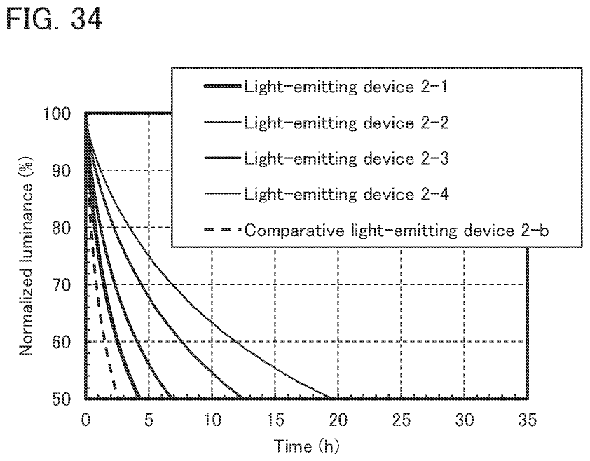

Driving tests of Light-emitting device 2-1, Light-emitting device 2-2, Light-emitting device 2-3, Light-emitting device 2-4, and Comparative light-emitting device 2-b at a constant current density of 50 mA/cm² were performed. The results are shown in FIG. 34. The results show that an increase in the concentration of 22′66′Ph-mmtBuDPhA2BANT improves the reliability while high emission efficiency is achieved. This means that by increasing the concentration of a guest in the light-emitting layer, excitation energy in the light-emitting layer can be efficiently converted into light emission of the guest and non-radiative deactivation can be inhibited. In other words, it is suggested that an increase in the concentration of the guest can prevent energy transfer from the host to the guest due to the Dexter mechanism and can increase the rate of the energy transfer from the host to the guest due to the Förster mechanism. Therefore, the light-emitting device including the compound of one embodiment of the present invention has high emission efficiency and high reliability.

### <<CV Measurement Results>>

Then, the electrochemical characteristics (oxidation reaction characteristics and reduction reaction characteristics) of 4,6mCzP2Pm and [Ir(ppz)₃] used for the light-emitting layers of the light-emitting elements were measured by cyclic voltammetry (CV) measurement. The measurement method is as follows.

An electrochemical analyzer (model number: ALS model 600A or 600C, manufactured by BAS Inc.) was used as a measurement apparatus. To prepare a solution for the CV measurement, dehydrated dimethylformamide (DMF) (manufactured by Sigma-Aldrich Inc., 99.8%, catalog No. 22705-6) was used as a solvent, tetra-n-butylammonium perchlorate (n-Bu₄NClO₄) (manufactured by Tokyo Chemical Industry Co., Ltd., catalog No. T0836) as a supporting electrolyte was dissolved at a concentration of 100 mmol/L, and the object to be measured was dissolved at a concentration of 2 mmol/L. A platinum electrode (PTE platinum electrode, manufactured by BAS Inc.) was used as a working electrode, another platinum electrode (Pt counter electrode for VC-3 (5 cm), manufactured by BAS Inc.) was used as an auxiliary electrode, and an Ag/Ag⁺ electrode (RE7 reference electrode for non-aqueous solvent, manufactured by BAS Inc.) was used as a reference electrode. Note that the measurement was performed at room temperature (20 to 25° C.). The scan speed in the CV measurement was fixed to 0.1 V/sec, and an oxidation potential Ea [V] and a reduction potential Ec [V] with respect to the reference electrode were measured. Ea is an intermediate potential of an oxidation-reduction wave, and Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Ea and LUMO level [eV]=−4.94−Ec.

According to the CV measurement results, the oxidation potential of 4,6mCzP2Pm was 0.95 V and the reduction potential was −2.06 V. The HOMO level of 4,6mCzP2Pm, which was calculated from the CV measurement, was −5.89 eV and the LUMO level was −2.88 eV. The oxidation potential of [Ir(ppz)₃] was 0.45 V and the reduction potential was −3.17 V. The HOMO level of Ir(ppz)₃, which was calculated from the CV measurement, was −5.39 eV and the LUMO level was −1.77 eV.

As described above, the LUMO level of 4,6mCzP2Pm is lower than the LUMO level of [Ir(ppz)₃], and the HOMO level of [Ir(ppz)₃] is higher than the HOMO level of 4,6mCzP2Pm. Thus, in the case where the compounds are used in a light-emitting layer, electrons and holes are efficiently injected into 4,6mCzP2Pm and [Ir(ppz)₃], respectively, so that 4,6mCzP2Pm and [Ir(ppz)₃] can form an exciplex. Furthermore, light emission energy of the EL spectrum of Comparative light-emitting device 2-b shown in FIG. 33 is close to an energy difference between the HOMO level of 4,6mCzP2Pm and the LUMO level of [Ir(ppz)₃], which also indicates that the light emission is derived from the exciplex formed by 4,6mCzP2Pm and [Ir(ppz)₃].

### Example 5

### Synthesis Example 3

In this example, a synthesis method of N,N-bis[3,5-bis(2-adamantyl)phenyl)-N,N-bis[3,5-bis(3,5-di-tert-butylphenyl)phenyl]-2,2′,6,6′-tetrakis(3,5-di-tert-butylphenyl)-9,9′-bianthracene-10,10′-diamine (abbreviation: 22′66′mmtBuPh-mmAdtBuDPhA2BANT-02), which is an organic compound represented by Structural Formula (103) of Embodiment 1, is described. A structure of 22′66′mmtBuPh-mmAdtBuDPhA2BANT-02 is shown below.

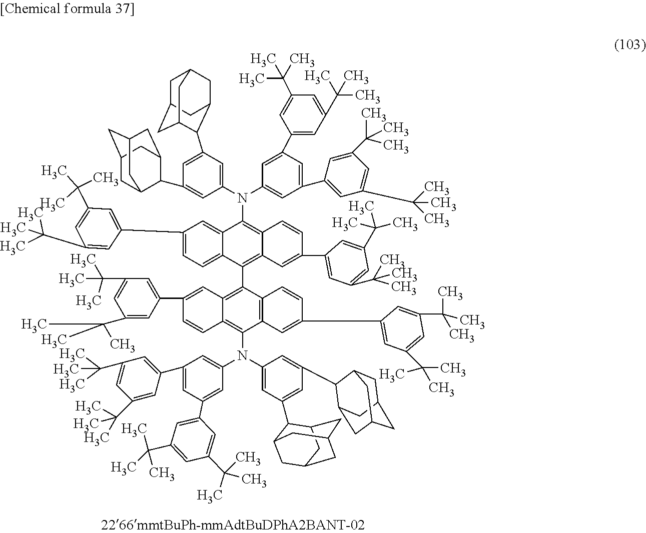 [SMILES: CC(C)(C)c1cc(-c2cc(-c3cc(C(C)(C)C)cc(C(C)(C)C)c3)cc(N(c3cc(C4C5CC6CC(C5)CC4C6)cc(C4C5CC6CC(C5)CC4C6)c3)c3c4ccc(-c5cc(C(C)(C)C)cc(C(C)(C)C)c5)cc4c(-c4c5cc(-c6cc(C(C)(C)C)cc(C(C)(C)C)c6)ccc5c(N(c5cc(-c6cc(C(C)(C)C)cc(C(C)(C)C)c6)cc(-c6cc(C(C)(C)C)cc(C(C)(C)C)c6)c5)c5cc(C6C7CC8CC(C7)CC6C8)cc(C6C7CC8CC(C7)CC6C8)c5)c5cc(-c6cc(C(C)(C)C)cc(C(C)(C)C)c6)ccc45)c4ccc(-c5cc(C(C)(C)C)cc(C(C)(C)C)c5)cc34)c2)cc(C(C)(C)C)c1]

The above compound 22′66′mmtBuPh-mmAdtBuDPhA2BANT-02 can be synthesized in a similar manner using 3,5-bis(2-adamantyl)-3′,5′-bis(3,5-di-tert-butylphenyl)diphenylamine instead of bis(3,5-di-tert-butylphenyl)amine used in Step 4 of Example 2 by the method in Synthesis Schemes (c-6) shown below. Note that 3,5-bis(2-adamantyl)-3′,5′-bis(3,5-di-tert-butylphenyl)diphenylamine can be synthesized by Schemes (c-1), (c-2), (c-3), (c-4), and (c-5) shown below.

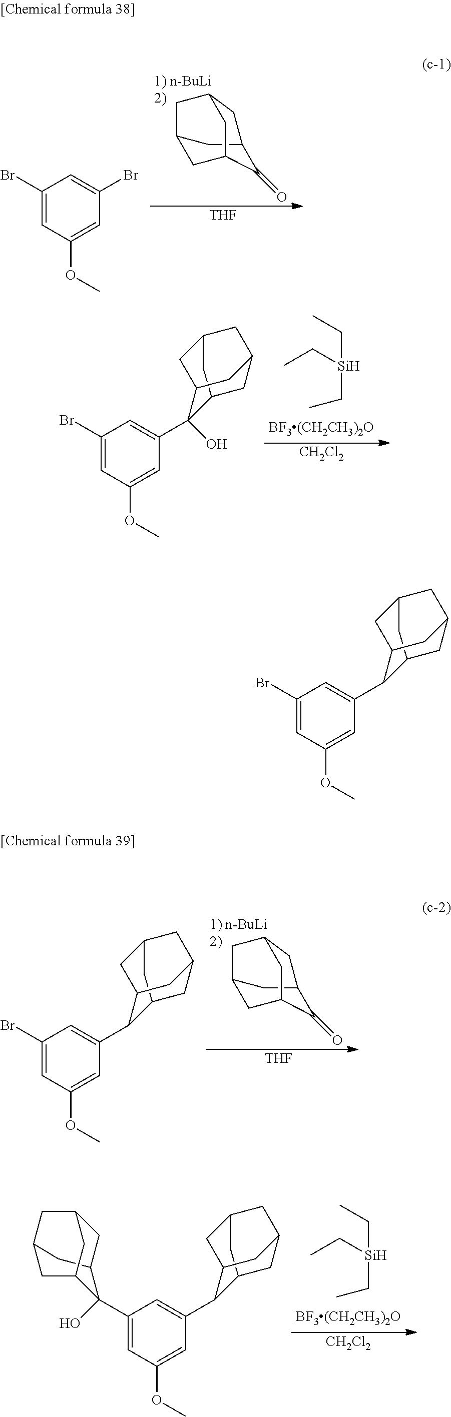

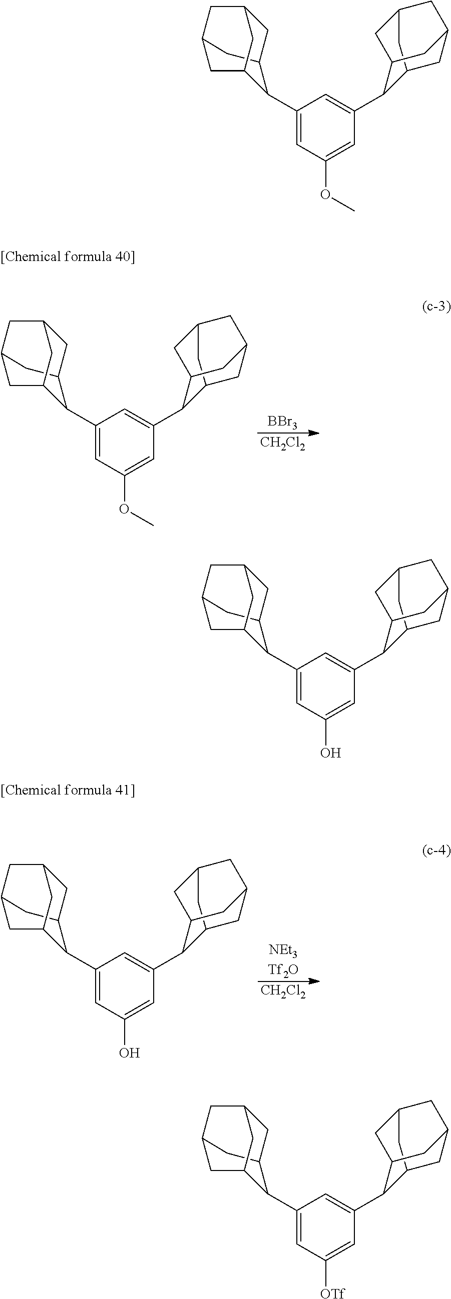

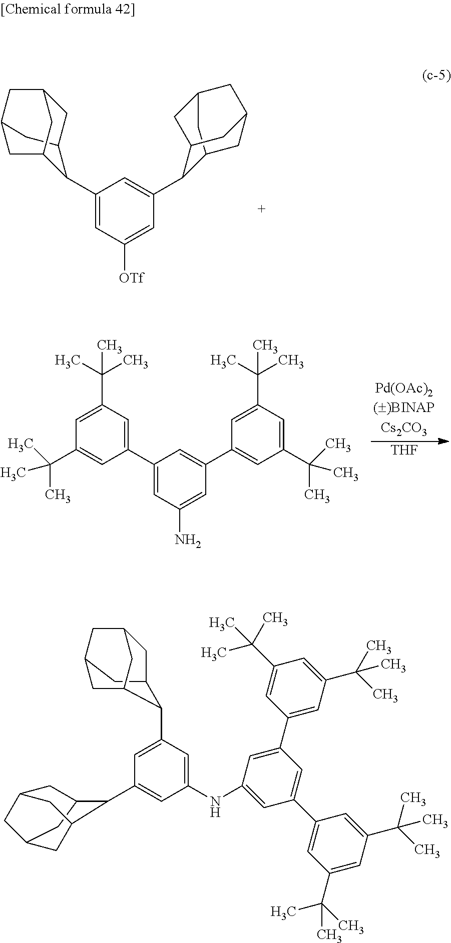 [SMILES: O=S(=O)(Oc1cc(C2C3CC4CC(C3)CC2C4)cc(C2C3CC4CC(C3)CC2C4)c1)C(F)(F)F ; CC(C)(C)c1cc(-c2cc(N)cc(-c3cc(C(C)(C)C)cc(C(C)(C)C)c3)c2)cc(C(C)(C)C)c1 ; "THF" ; CC(C)(C)c1cc(-c2cc(Nc3cc(C4C5CC6CC(C5)CC4C6)cc(C4C5CC6CC(C5)CC4C6)c3)cc(-c3cc(C(C)(C)C)cc(C(C)(C)C)c3)c2)cc(C(C)(C)C)c1]

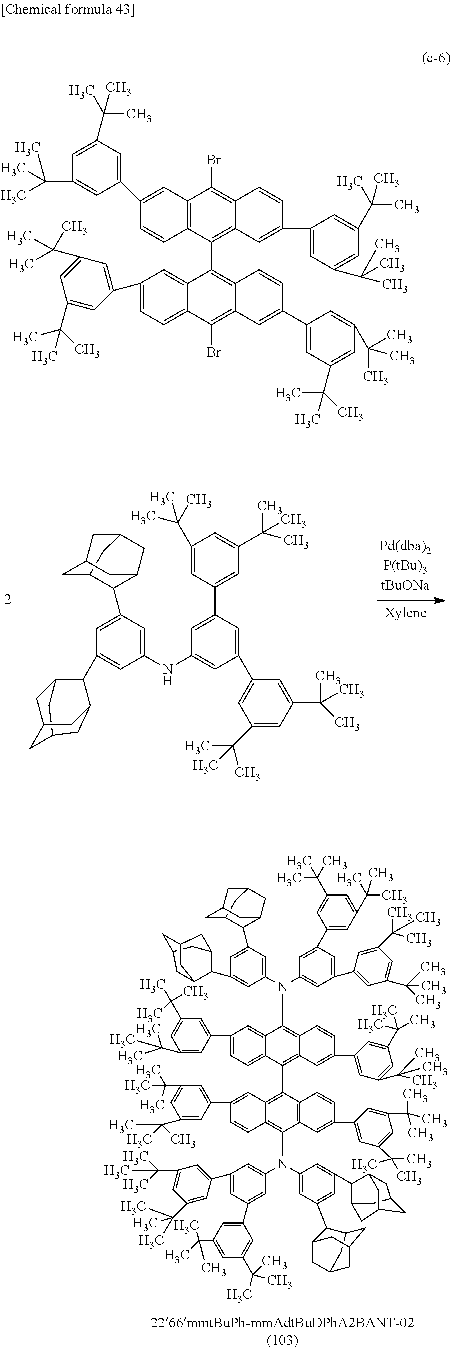

Thus, the compound of one embodiment of the present invention represented by Structure Formula (103), 22′66′mmtBuPh-mmAdtBuDPhA2BANT-02, can be obtained.

### REFERENCE NUMERALS

101: first electrode, 102: second electrode, 103: EL layer, 103a, 103b: EL layer, 104: charge generation layer, 111, 111a, 111b: hole-injection layer, 112, 112a, 112b: hole-transport layer, 113, 113a, 113b: light-emitting layer, 114, 114a, 114b: electron-transport layer, 115, 115a, 115b: electron-injection layer, 124: fluorescent substance, 124a: luminophore, 131: protecting group, 132: compound (fluorescent substance), 132a: luminophore, 132b: protecting group, 200R, 200G, 200B: optical distance, 201: first substrate, 202: transistor (FET), 203R, 203G, 203B, 203W: light-emitting device, 204: EL layer, 205: second substrate, 206R, 206G, 206B: color filter, 206R′, 206G′, 206B′: color filter, 207: first electrode, 208: second electrode, 209: black layer (black matrix), 210R, 210G: conductive layer, 301: first substrate, 302: pixel portion, 303: driver circuit portion (source line driver circuit), 304a, 304b: driver circuit portion (gate line driver circuit), 305: sealing material, 306: second substrate, 307: lead wiring, 308: FPC, 309: FET, 310: FET, 311: FET, 312: FET, 313: first electrode, 314: insulator, 315: EL layer, 316: second electrode, 317: light-emitting device, 318: space, 900: substrate, 901: first electrode, 902: EL layer, 903: second electrode, 911: hole-injection layer, 912: hole-transport layer, 913: light-emitting layer, 914: electron-transport layer, 915: electron-injection layer, 4000: lighting device, 4001: substrate, 4002: light-emitting device, 4003: substrate, 4004: first electrode, 4005: EL layer, 4006: second electrode, 4007: electrode, 4008: electrode, 4009: auxiliary wiring, 4010: insulating layer, 4011: sealing substrate, 4012: sealing material, 4013: desiccant, 4015: diffusion plate, 4200: lighting device, 4201: substrate, 4202: light-emitting device, 4204: first electrode, 4205: EL layer, 4206: second electrode, 4207: electrode, 4208: electrode, 4209: auxiliary wiring, 4210: insulating layer, 4211: sealing substrate, 4212: sealing material, 4213: barrier film, 4214: planarization film, 4215: diffusion plate, 5101: light, 5102: wheel, 5103: door, 5104: display portion, 5105: handle, 5106: gear lever, 5107: seat, 5108: inner rearview mirror, 5109: windshield, 7000: housing, 7001: display portion, 7002: second display portion, 7003: speaker, 7004: LED lamp, 7005: operation key, 7006: connection terminal, 7007: sensor, 7008: microphone, 7009: switch, 7010: infrared port, 7011: recording medium reading portion, 7012: support portion, 7013: earphone, 7014: antenna, 7015: shutter button, 7016: image receiving portion, 7018: stand, 7020: camera, 7021: external connection portion, 7022, 7023: operation button, 7024: connection terminal, 7025: band, 7026: microphone, 7027: icon indicating time, 7028: another icon, 7029: sensor, 7030: speaker, 7052, 7053, 7054: information, 9310: portable information terminal, 9311: display portion, 9312: display region, 9313: hinge, 9315: housing

1. A compound represented by General Formula (G1), 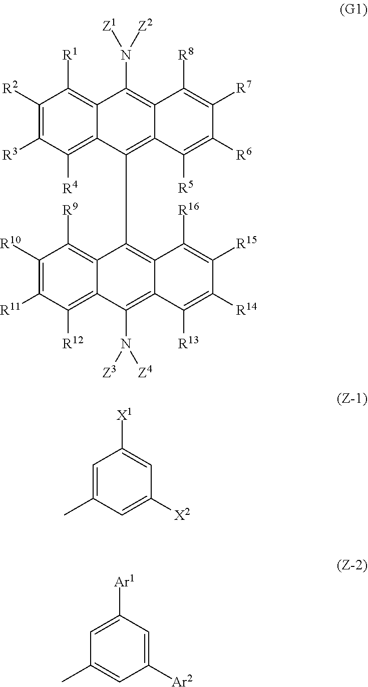
wherein, each of Z¹ to Z⁴ independently has a structure represented by General formula (Z-1) or General formula (Z-2),
wherein in General Formula (Z-1), each of X¹ and X² independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms,
wherein each of Ar¹ and Ar² independently represents an aromatic hydrocarbon group having 6 to 13 carbon atoms, and at least one of Ar¹ and Ar² comprises a substituent that is the same as X¹, and
wherein each of R¹ to R¹⁶ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

2. The compound according to claim 1, wherein General Formula (G1) is represented by General Formula (G2), 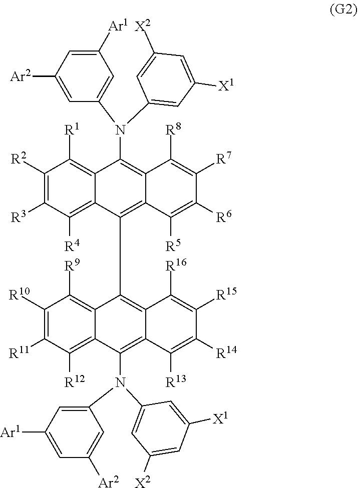
wherein each of X¹ and X² independently represents any one of an alkyl group having 3 to 10 carbon atoms, a substituted or unsubstituted cycloalkyl group having 3 to carbon atoms, a cycloalkyl group with a bridge structure having 7 to 10 carbon atoms, and a trialkylsilyl group having 3 to 12 carbon atoms.

3. The compound according to claim 2, wherein General Formula (G2) is represented by General Formula (G3), 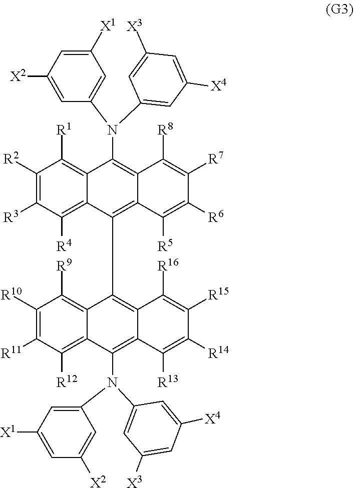
wherein each of X³ and X⁴ independently represents any one of an alkyl group having 3 to 10 carbon atoms, a substituted or unsubstituted cycloalkyl group having 3 to 10 carbon atoms, a cycloalkyl group with a bridge structure having 7 to 10 carbon atoms, and a trialkylsilyl group having 3 to 12 carbon atoms.

4. A compound represented by General Formula (G4), 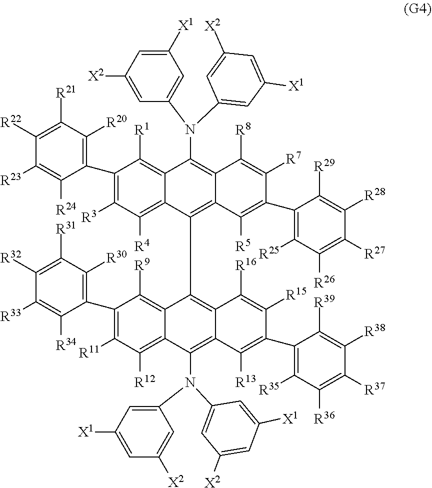
wherein each of X¹ and X² independently represents any one of an alkyl group having 3 to 10 carbon atoms, a substituted or unsubstituted cycloalkyl group having 3 to carbon atoms, a cycloalkyl group with a bridge structure having 7 to 10 carbon atoms, and a trialkylsilyl group having 3 to 12 carbon atoms, and
wherein each of R¹, R³ to R⁵, R⁷ to R⁹, R¹¹ to R¹³, R¹⁵ to R¹⁶, and R²⁰ to R³⁹ independently represents any one of hydrogen, an alkyl group having 3 to 10 carbon atoms, a substituted or unsubstituted cycloalkyl group having 3 to 10 carbon atoms, a trialkylsilyl group having 3 to 12 carbon atoms, and a substituted or unsubstituted aryl group having 6 to 25 carbon atoms.

5. The compound according to claim 4, wherein General Formula (G4) is represented by Structural Formula (100) or Structural Formula (101),

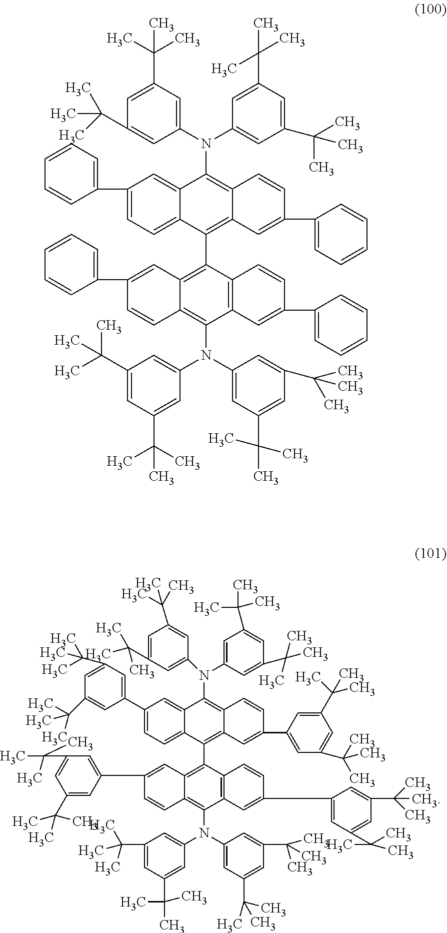

6. A light-emitting device comprising an EL layer between a pair of electrodes,
wherein the EL layer comprises the compound according to claim 1.

7. A light-emitting device comprising an EL layer between a pair of electrodes,
wherein the EL layer comprises a light-emitting layer, and
wherein the light-emitting layer comprises the compound according to claim 1.

8. A light-emitting device comprising an EL layer between a pair of electrodes,
wherein the EL layer comprises a light-emitting layer, and
wherein the light-emitting layer comprises the compound according to claim 1 and a phosphorescent material.

9. A light-emitting device comprising an EL layer between a pair of electrodes,
wherein the EL layer comprises a light-emitting layer, and
wherein the light-emitting layer comprises the compound according to claim 1 and a TADF material.

10. A light-emitting apparatus comprising:
the light-emitting device according to claim 6; and
at least one of a transistor and a substrate.

11. An electronic device comprising:
the light-emitting apparatus according to claim 10; and
at least one of a microphone, a camera, an operation button, an external connection portion, and a speaker.

12. A lighting device comprising:
the light-emitting device according to claim 6; and
at least one of a housing, a cover, and a support.

13. A light-emitting device comprising an EL layer between a pair of electrodes,
wherein the EL layer comprises the compound according to claim 4.

14. A light-emitting device comprising an EL layer between a pair of electrodes,
wherein the EL layer comprises a light-emitting layer, and
wherein the light-emitting layer comprises the compound according to claim 4.

15. A light-emitting device comprising an EL layer between a pair of electrodes,
wherein the EL layer comprises a light-emitting layer, and
wherein the light-emitting layer comprises the compound according to claim 4 and a phosphorescent material.

16. A light-emitting device comprising an EL layer between a pair of electrodes,
wherein the EL layer comprises a light-emitting layer, and
wherein the light-emitting layer comprises the compound according to claim 4 and a TADF material.

17. A light-emitting apparatus comprising:
the light-emitting device according to claim 13; and
at least one of a transistor and a substrate.

18. An electronic device comprising:
the light-emitting apparatus according to claim 17; and
at least one of a microphone, a camera, an operation button, an external connection portion, and a speaker.

19. A lighting device comprising:
the light-emitting device according to claim 13; and
at least one of a housing, a cover, and a support.
